SEQUENCE LISTING (26 sequences)

SEQ ID NO: 1 - protein, 5 aa, Artificial Sequence
  from human Fab library
SYIMM

SEQ ID NO: 2 - protein, 17 aa, Artificial Sequence
  from human Fab library
SIYPSGGITFYADTVKG

SEQ ID NO: 3 - protein, 11 aa, Artificial Sequence
  from human Fab library
IKLGTVTTVDY

SEQ ID NO: 4 - protein, 14 aa, Artificial Sequence
  from human Fab library
TGTSSDVGGYNYVS

SEQ ID NO: 5 - protein, 7 aa, Artificial Sequence
  from human Fab library
DVSNRPS

SEQ ID NO: 6 - protein, 10 aa, Artificial Sequence
  from human Fab library
SSYTSSSTRV

SEQ ID NO: 7 - protein, 216 aa, Artificial Sequence
  from human Fab library
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 8 - protein, 607 aa, Artificial Sequence
  Synthetic peptide
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 9 - protein, 592 aa, Homo sapiens
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 10 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 11 - protein, 136 aa, Homo sapiens
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 12 - protein, 117 aa, Homo sapiens
GAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 13 - protein, 115 aa, Homo sapiens
VKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 14 - protein, 446 aa, Artificial Sequence
  Description of Artificial Sequence Synthetic polypeptide
QVQLVQSGAEVKKPGASVKVSCKASGYTFTSYWMHWVRQAPGQGLEWMGRIGPNSGFTSYNEKFKNRVTMTRDTSTSTVYMELSSLRSEDTAVYYCARGGSSYDYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 218 aa, Artificial Sequence
  Description of Artificial Sequence Synthetic polypeptide
DIVLTQSPASLAVSPGQRATITCRASESVSIHGTHLMHWYQQKPGQPPKLLIYAASNLESGVPARFSGSGSGTDFTLTINPVEAEDTANYYCQQSFEDPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 450 aa, Artificial Sequence
  Description of Artificial Sequence Synthetic polypeptide
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 584 aa, Artificial Sequence
  Description of Artificial Sequence Synthetic polypeptide
QVQLVQSGAEVKKPGASVKVSCKASGYTFTSYWMHWVRQAPGQGLEWMGRIGPNSGFTSYNEKFKNRVTMTRDTSTSTVYMELSSLRSEDTAVYYCARGGSSYDYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSGGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 18 - protein, 587 aa, Artificial Sequence
  Description of Artificial Sequence Synthetic polypeptide
QVQLVQSGAEVKKPGASVKVSCKASGYTFTSYWMHWVRQAPGQGLEWMGRIGPNSGFTSYNEKFKNRVTMTRDTSTSTVYMELSSLRSEDTAVYYCARGGSSYDYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSGGGGSGVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 19 - protein, 5 aa, Mus musculus
SYWMH

SEQ ID NO: 20 - protein, 17 aa, Artificial sequence
  Description of Artificial Sequence Synthetic polypeptide
  VARIANT 3: His or Gly
  VARIANT 8: Gly or Phe
RIXPNSGXTSYNEKFKN

SEQ ID NO: 21 - protein, 10 aa, Mus musculus
GGSSYDYFDY

SEQ ID NO: 22 - protein, 15 aa, Mus musculus
RASESVSIHGTHLMH

SEQ ID NO: 23 - protein, 7 aa, Mus musculus
AASNLES

SEQ ID NO: 24 - protein, 9 aa, Mus musculus
QQSFEDPLT

SEQ ID NO: 25 - protein, 110 aa, Artificial Sequence
  From human Fab library
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 26 - protein, 120 aa, Artificial Sequence
  From human Fab library
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

(19) United States
(12) Patent Application Publication
(10) Pub. No.: US 2024/0279338 A1
(43) Pub. Date: Aug. 22, 2024

(54) Combination Treatment of Cancer

(71) Applicants: Ares Trading S.A., Aubonne (CH); GLAXOSMITHKLINE INTELLECTUAL PROPERTY (NO. 4) LTD., Brentford Middlesex (GB)

(72) Inventors: Rinat Zaynagetdinov, Billerica, MA (US); Kalyan Chakravarthy Nallaparaju, Billerica, MA (US); Natalya Belousova, Billerica, MA (US); Yan Lan, Billerica, MA (US)

(21) Appl. No.: 18/567,216

(22) PCT Filed: Jun. 7, 2022
(86) PCT No.: PCT/EP2022/065408

§ 371 Date: Dec. 5, 2023

Related U.S. Application Data
(60) Provisional application No. 63/197,793, filed on Jun. 7, 2021.

(51) Int. Cl. C07K 16/28; A61K 31/4439; A61K 38/00; A61K 39/395; A61P 35/00; C07K 14/71; C07K 16/22

(52) U.S. Cl. CPC C07K 16/2827; A61K 31/4439; A61K 39/3955; A61P 35/00; C07K 14/71; C07K 16/22; A61K 38/00; C07K 2319/30

(57) ABSTRACT

The present invention relates to combination therapies useful for the treatment of cancer. In particular, the invention relates to the combined use of a PD-1 inhibitor, a TGFβ inhibitor, and an adenosine inhibitor to treat cancer.

### FIELD OF INVENTION

The present invention relates to the treatment of cancer. In particular, the invention relates to a combination of compounds for inhibiting PD-1, TGFβ and adenosine for use in treating cancer.

### BACKGROUND OF THE INVENTION

Adenosine is an ubiquitous modulator of numerous physiological activities, particularly within the cardiovascular, nervous and immune systems. Adenosine is related both structurally and metabolically to the bioactive nucleotides adenosine triphosphate (ATP), adenosine diphosphate (ADP), adenosine monophosphate (AMP) and cyclic adenosine monophosphate (cAMP), to the biochemical methylating agent S-adenosyl-L-methione (SAM) and structurally to the coenzymes NAD, FAD and coenzym A and to RNA.

Exhibit A Under physiological conditions, endogenous adenosine is present at low (30 to 300 nM) concentrations in the extracellular space of normal tissues (Fredholm et al., Drug Dev Res 52:274-282 (2001)). However, increased energy consumption or hypoxia under metabolically stressful conditions, including inflammation and cancer, result in dramatic increases in extracellular adenosine concentrations (Cronstein, B. N. J Appl Physiol, 1994; 76(1), 5-13; Ohta, A. Front Immunol, 2016; 7(109), 1-11). The key drivers for adenosine accumulation in the extracellular compartment are CD39 and CD73—the enzymes which catalyze the degradation of adenosine triphosphate (ATP) and adenosine diphosphate (ADP) to adenosine monophosphate (AMP) and AMP to adenosine, respectively. In agreement, adenosine concentrations are reduced in mice lacking CD73 (Grenz, A. et al. J Am Soc Nephrol, 2007; 18, 833-845). Adenosine levels may also increase due to the uncontrolled damage to cells that can lead to passive leakage of adenosine into extracellular space. Furthermore, in inflamed tissues, increased adenosine may be associated with its release from inflammatory effector cells, including activated polymorphonuclear cells (Lennon, P. F. et al. J Exp Med, 1998; 188(8), 1433-1443).

The regulatory functions of adenosine are mediated through G protein-coupled receptors (GPCRs) of the adenosine family, which include A₁, A_{2A}, A_{2B}, and A₃ (Fredholm, B. B. et al. Pharmacol Rev, 2001; 53(4), 527-552). These adenosine receptor subtypes are classified based on their effect on adenylate cyclase. Highly adenosine-sensitive A₁ and A₃ receptors (300 nM and 100 nM, respectively) inhibit adenylate cyclase via the Gi subunit of GPCRs. In contrast, A_{2A} and A_{2B} adenosine receptors exhibit lower affinities to adenosine (700 nM and 24 μM, respectively) and therefore mediate signaling at higher adenosine concentrations. Signaling downstream of A_{2A}, as well as A_{2B}, is mediated through the Gs subunit of GPCR and induces phosphorylation of cyclic adenosine monophosphate (cAMP) response element binding protein (CREB) (Nemeth, Z. H. et al. BBRC, 2003; 312, 883-888; Gao, Z. G. et al. Biochemical Pharmacology, 2018; 151, 201-213), leading to suppression of immune cell activation (Penix, L. A. et al. J Biol Chem, 1996; 271, 31964-31972). Importantly, A_{2B} adenosine receptors can also signal through the Gq subunit of GPCR and activate other pro-tumorigenic pathways, including phospholipase Cr, protein kinase C (PKC), and p38 MAP kinase (Schulte, G., Fredholm, B. B. Cellular Signalling, 2003; 15, 813-827).

The A_{2A} adenosine receptor has been recognized as the key receptor for adenosine-driven suppression of T cell, natural killer (NK) cell, and myeloid cell functions (Cekic, C., et al. Cancer Res, 2014; 74(24), 7250-7259). A_{2A} may support adenosine-driven immunosuppression either through direct suppression of immune cell function or through recruitment of other immunoregulatory cells, including regulatory T cells (Tregs). Together, these findings suggest that inhibition of the A_{2A} adenosine receptor may protect anti-tumor immune responses from adenosine-driven immunosuppression. However, the A_{2A} receptor shares signaling with the lower affinity A_{2B} adenosine receptor through Gs-subunit activation of adenylate cyclase. In this regard, the A_{2B} receptor can compensate for inhibition of A_{2A} in an adenosine-rich tumor microenvironment (TME). Moreover, blocking A_{2B} is expected to address Gq-mediated mechanistic aspects of adenosine-mediated tumor promotion, leading to reduced tumor neovascularization through decreased production of vascular endothelial growth factor (VEGF) by myeloid cells and tumor cells (Sorrentino, C. et al. Oncotarget, 2015; 6(29), 27478-27489). In addition, blocking A_{2B} is expected to support vascular permeability that may promote infiltration of leukocytes into tumors (Eckle, T. et al. Blood, 2008; 111(4), 2024-2035). Furthermore, inhibition of A_{2B} is expected to prevent accumulation of immune-suppressive precursors of dendritic cells (DCs) (Novitskiy, S. V. et al. Blood, 2008; 112(5), 1822-1831) and polarization of pro-tumorigenic M2 macrophage cells (Csoka, B. et al. FASEB, 2012; 26(1), 376-386). Taken together, inhibition of A_{2B} adenosine receptors, in addition to inhibition of A_{2A} adenosine receptors, is expected to provide more robust protection from adenosine-driven tumor promotion.

Monoclonal antibodies that block the interaction of programmed death 1 (PD-1) to its ligand PD-L1, such as the anti-PD-L1 antibody avelumab, can enhance the immune response against cancer and are among the most promising immune checkpoint antagonists. However, although compelling clinical efficacy results have led to several new approvals of anti-PD(L)-1 drugs, not all patients experience durable clinical responses to current immune checkpoint antagonists. In fact, patients with certain tumor types, including prostate, colorectal, and pancreatic cancers, show little response to anti-PD(L)-1 monotherapies in clinical trials. Combination therapy with other checkpoint antagonists may be a necessary strategy to elicit synergistic anti-tumor activity in nonresponsive patients, or to enhance anti-tumor immune responses in partial responders. In preclinical mouse models, co-inhibition of the adenosine A_{2A} receptor and PD-1 has demonstrated remarkable synergy in enhancing tumor growth inhibition relative to monotherapies (Beavis, P. A. et al. Cancer Immunol Res, 2015; 3(5), 506-517; Willingham, S. B., et al. Cancer Immunol Res, 2018; 6(10), 1136-1149).

WO 2015/118175 describes a bifunctional fusion protein composed of the extracellular domain of the tumor growth factor beta receptor type II (TGFβRII) to function as a TGF-β “trap” fused to a human IgG1 antibody blocking PD-L1. Specifically, the protein is a heterotetramer, consisting of the two immunoglobulin light chains of an anti-PD-L1 antibody, and two heavy chains each comprising a heavy chain of the anti-PD-L1 antibody genetically fused via a flexible glycine-serine linker to the extracellular domain of the human TGFβRII (see FIG. 1). This fusion molecule is designed to target both the PD-L1 pathway and the TGFβ pathway to counteract immunosuppression in the tumor microenvironment.

There remains a need to develop novel therapeutic options for the treatment of cancers. Furthermore, there is a need for therapies having greater efficacy than existing therapies.

### SUMMARY OF THE INVENTION

The present invention arises out of the discovery that a therapeutic benefit in the treatment of cancer can be achieved by combining compounds which inhibit the PD-1, TGFβ and adenosine signaling pathways. Such therapeutic benefit is particularly pronounced in the treatment of cancer with high adenosine-mediated signaling, such as a cancer that is CD73 positive or adenosine-rich.

Thus, in a first aspect, the present disclosure provides a PD-1 inhibitor, a TGFβ inhibitor and an adenosine inhibitor, such as an adenosine A_{2A} and/or A_{2B} receptor inhibitor, for use in a method of treating a cancer in a subject, for use in inhibiting tumor growth or progression in a subject who has malignant tumors, for use in inhibiting metastasis of malignant cells in a subject, for use in decreasing the risk of metastasis development and/or metastasis growth in a subject, or for use in inducing tumor regression in a subject who has malignant cells, wherein the use comprises administering said compounds to the subject.

The present disclosure also provides the use of a PD-1 inhibitor, a TGFβ inhibitor and an adenosine inhibitor, such as an adenosine A_{2A} and/or A_{2B} receptor inhibitor, for the manufacture of a medicament for treating a cancer in a subject, for inhibiting tumor growth or progression in a subject who has malignant tumors, for inhibiting metastasis of malignant cells in a subject, for decreasing the risk of metastasis development and/or metastasis growth in a subject, or for inducing tumor regression in a subject who has malignant cells.

In another aspect, the present disclosure provides a method of treating a cancer in a subject, a method of inhibiting tumor growth or progression in a subject who has malignant tumors, a method of inhibiting metastasis of malignant cells in a subject, a method of decreasing the risk of metastasis development and/or metastasis growth in a subject, or a method of inducing tumor regression in a subject who has malignant cells, wherein the method comprises administering a PD-1 inhibitor, a TGFβ inhibitor and an adenosine inhibitor, such as an adenosine A_{2A} and/or A_{2B} receptor inhibitor, to the subject.

In a further aspect, the disclosure relates to a method for advertising treatment with a PD-1 inhibitor, a TGFβ inhibitor, and an adenosine inhibitor, such as an adenosine A_{2A} and/or A_{2B} receptor inhibitor, comprising promoting, to a target audience, the use of the combination for treating a subject with a cancer, e.g., based on PD-L1 expression in samples, such as tumor samples, taken from the subject.

Provided herein is also a pharmaceutical composition comprising a PD-1 inhibitor, a TGFβ inhibitor, and an adenosine inhibitor, such as an adenosine A_{2A} and/or A_{2B} receptor inhibitor, and at least a pharmaceutically acceptable excipient or adjuvant. In one embodiment, the PD-1 inhibitor and TGFβ inhibitor are fused in such pharmaceutical composition. The PD-1 inhibitor, the TGFβ inhibitor and the adenosine inhibitor are provided in a single or separate unit dosage forms.

In a further aspect, the present disclosure relates to a kit comprising a PD-1 inhibitor, a TGFβ inhibitor, and an adenosine inhibitor, such as an adenosine A_{2A} and/or A_{2B} receptor inhibitor, and a package insert comprising instructions for using said compounds, to treat or delay progression of a cancer in a subject. In a further aspect, the invention relates to a kit comprising a PD-1 inhibitor and a package insert comprising instructions for using the PD-1 inhibitor, a TGFβ inhibitor, and an adenosine inhibitor, such as an adenosine A_{2A} and/or A_{2B} receptor inhibitor, to treat or delay progression of a cancer in a subject. In a further aspect, the invention relates to a kit comprising a TGFβ inhibitor and a package insert comprising instructions for using the TGFβ inhibitor, a PD-1 inhibitor, and an adenosine inhibitor, such as an adenosine A_{2A} and/or A_{2B} receptor inhibitor, to treat or delay progression of a cancer in a subject. In a further aspect, the invention relates to a kit comprising an adenosine inhibitor, such as an adenosine A_{2A} and/or A_{2B} receptor inhibitor, and a package insert comprising instructions for using the adenosine inhibitor, a PD-1 inhibitor, and a TGFβ inhibitor to treat or delay progression of a cancer in a subject. In a further aspect, the invention relates to a kit comprising an anti-PD(L)1:TGFβRII fusion protein and a package insert comprising instructions for using the anti-PD(L)1:TGFβRII fusion protein and an adenosine inhibitor, such as an adenosine receptor inhibitor, to treat or delay progression of a cancer in a subject. The compounds of the kit may be comprised in one or more containers. The instructions can state that the medicaments are intended for use in treating a subject having a cancer that tests positive for PD-L1 expression by an immunohistochemical (IHC) assay.

In certain embodiments, the PD-1 inhibitor and the TGFβ inhibitor are fused. In one embodiment, the fusion molecule is an anti-PD(L)1:TGFβRII fusion protein. In one embodiment, the fusion molecule is an anti-PD-L1:TGFβRII fusion protein. In one embodiment, the amino acid sequence of the anti-PD-L1:TGFβRII fusion protein corresponds to the amino acid sequence of bintrafusp alfa.

### BRIEF DESCRIPTION OF THE FIGURES

FIG. 1 shows the amino acid sequence of bintrafusp alfa. (A) SEQ ID NO: 8 represents the heavy chain sequence of bintrafusp alfa. The CDRs having the amino acid sequences of SEQ ID NOs: 1, 2 and 3 are underlined. (B) SEQ ID NO: 7 represents the light chain sequence of bintrafusp alfa. The CDRs having the amino acid sequences of SEQ ID NOs: 4, 5 and 6 are underlined.

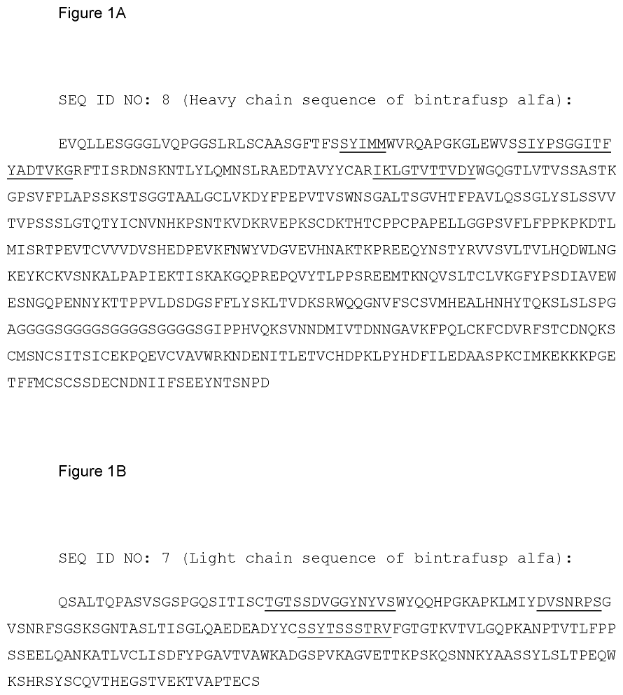

FIG. 2 shows an exemplary structure of an anti-PD-L1:TGFβRII fusion protein.

FIG. 3: Concentration of AMP and adenosine in 4T1 and MC38 murine tumors in vivo or CD73 expression by 4T1, MC38, E0771, EMT6, MC38, H22 and MBT2 tumor cells. Female mice were inoculated with either 5×10⁴ 4T1 cells into the right mammary fat pad or 1×10⁶ MC38 cells subcutaneously. Tumors were collected and analyzed for (A) AMP or (B) adenosine concentration using LC-MS-MS mass spectrometry analysis. The average concentrations for AMP and adenosine were 296.5±250.5 (±SD) nM/g and 210.2±158.2 nM/g for 4T1 model, respectively, and 850.4±215.6 nM/g and 87.2±51.9 nM/g for MC38 model, respectively. Each dot represents an individual mouse and the line represents median values for metabolites in each model. Expression of CD73 by murine (C) 4T1 mammary carcinoma, (D) MC38 colon carcinoma, (E) E0771 and (F) mammary carcinoma, (G) H22 hepatocellular carcinoma and (H) MBT2 bladder tumor cells assessed by flow cytometry.

FIG. 4: Bintrafusp alfa increases expression of A_{2B} in CD73h adenosine-rich 4T1 tumor model, but not in CD73^{low} adenosine-low MC38 tumor model. For the 4T1 tumor model, female BALB/c mice were inoculated with 2×10⁵ 4T1 cells into the right mammary fat pad. For the MC38 tumor model, female C57BL/6 mice were inoculated subcutaneously in the right flank with MC38 cells (1×10⁶). Animals in both studies were randomized into the groups and treatments started when the average tumor size reached approximately 100-150 mm³. For both models, there were 15 mice in each group. The animals were treated with isotype control (20 mg/kg, iv) or bintrafusp alfa (24.6 mg/kg, iv) on days 0, 1, and 2. Tumors were harvested on Day 6 post treatment start, flash frozen, and used for RNASeq analysis. Gene expression levels of (A) NT5E, (B) ADORA2A (A_{2A}), and (C) ADORA2B (A_{2B}) are presented as mean±SEM. P-values were calculated using two-way ANOVA.

FIG. 5: Compound A and bintrafusp alfa showed combination effect in the CD73h, adenosine-rich 4T1 tumor model. Female BALB/c mice were inoculated with 5×10⁴ 4T1 cells into the right mammary fat pad and were treated with Compound A (300 mg/kg po, bid), bintrafusp alfa (24.6 mg/kg iv, days 0, 3, 6), Compound A+bintrafusp alfa, when average tumor volume reached approximately 60 mm³. Vehicle (po, bid) and isotype control antibody injections (20 mg/kg iv, days 0, 3, 6) were used as controls. (A) Average tumor volumes with SEM, and (B) individual tumor volumes are presented. Tumor volume data were analyzed using two-way ANOVA followed by Tukey's multiple comparison test.

FIG. 6: Combination of Compound A and bintrafusp alfa enhanced anti-tumor efficacy in the CD73^{hi} EMT6 tumor model compared to either monotherapy. Female BALB/c mice were inoculated with 2.5×10⁵ EMT6 cells into the right mammary fat pad and were treated with Compound A (300 mg/kg po, BID), bintrafusp alfa (24.6 mg/kg iv, days 0, 3, 6), Compound A+bintrafusp alfa, when average tumor volume reached approximately 60 mm³. Vehicle (po, BID) and isotype control antibody injections (20 mg/kg iv, days 0, 3, 6) were used as controls. (A) Average tumor volumes with SEM, and (B) individual tumor volumes are presented. Tumor volume data were analyzed using two-way ANOVA followed by Tukey's multiple comparison test.

FIG. 7: Compound A and bintrafusp alfa showed combination anti-tumor activity in the CD73^{hi} E0771 tumor model. Female C57BL/6 mice were inoculated with 1.5×10⁵ E0771 cells into the right mammary fat pad and were treated with Compound A (300 mg/kg po, BID), bintrafusp alfa (8.2 mg/kg iv, days 0, 3, 6), Compound A+bintrafusp alfa, when average tumor volume reached approximately 75 mm³. Vehicle (po, BID) and isotype control antibody injections (6.65 mg/kg iv, days 0, 3, 6) were used as controls. (A) Average tumor volumes with SEM, and (B) individual tumor volumes are presented. Tumor volume data were analyzed using two-way ANOVA followed by Tukey's multiple comparison test.

FIG. 8: Compound A does not show combination anti-tumor activity with bintrafusp alfa in the CD73^{low} MC38 tumor model. Female C57BL/6 mice were inoculated with 1×10⁶ MC38 cells in the right lower flank and were treated with Compound A (300 mg/kg po, BID), bintrafusp alfa (24.6 mg/kg iv, days 0, 3, 6), Compound A+bintrafusp alfa, when average tumor volume reached approximately 70 mm³. Vehicle (po, BID) and isotype control antibody injections (20 mg/kg iv, days 0, 3, 6) were used as controls. (A) Average tumor volumes with SEM, and (B) individual tumor volumes are presented. Tumor volume data were analyzed using two-way ANOVA followed by Tukey's multiple comparison test.

FIG. 9: Combination of Compound A and bintrafusp alfa does not enhance anti-tumor efficacy in the CD73^{low} H22 tumor model compared to monotherapies. Female BALB/c mice were inoculated with 1×10⁶ H22 cells in the right upper flank and were treated with Compound A (300 mg/kg po, BID), bintrafusp alfa (24.6 mg/kg iv, days 0, 3, 6), Compound A+bintrafusp alfa, when average tumor volume reached approximately 55 mm3. Vehicle (po, BID) and isotype control antibody injections (20 mg/kg iv, days 0, 3, 6) were used as controls. (A) Average tumor volumes with SEM, and (B) individual tumor volumes are presented. Tumor volume data were analyzed using two-way ANOVA followed by Tukey's multiple comparison test.

FIG. 10: Combination of Compound A and bintrafusp alfa does not enhance anti-tumor efficacy in the CD73^{low} MBT2 tumor model compared to monotherapies. Female C3H mice were inoculated with 1×10⁶ MBT2 cells in the right upper flank and were treated with Compound A (300 mg/kg po, BID), bintrafusp alfa (24.6 mg/kg iv, days 0, 3, 6), Compound A+bintrafusp alfa, when average tumor volume reached approximately 53 mm³. Vehicle (po, BID) and isotype control antibody injections (20 mg/kg iv, days 0, 3, 6) were used as controls. (A) Average tumor volumes with SEM, and (B) individual tumor volumes are presented. Tumor volume data were analyzed using two-way ANOVA followed by Tukey's multiple comparison test.

FIG. 11: Combination of Compound A and bintrafusp alfa support significant rescue of IFNγ production from human T cells co-cultured with MDA-MB-231 tumor cells suppressed by NECA. EBV-specific T cells were pre-incubated in round bottom 96-well plate with Compound A (100 nM) or DMSO control and/or 1 μg/ml of either bintrafusp alfa or isotype control (hIgG1 inactive anti-PD-L1) antibodies for 15 minutes. Then 10 μM NECA was added to the corresponding groups and 1.3×10⁴ of T cells in 100 μl volume were transferred per well of 96-well flat bottom plate with 2.6×10⁴ cells/well of MDA-MB-231 tumor cells pre-loaded with EBV peptide (30 ng/ml, CLGGLLTMV, 21^{st} Century) in 100 μl of RPM11640 media, supplemented with 10% FBS. The final ratio for T cells:MDA-MB-231 tumor cells=0.5:1. Cell culture supernatants were collected after 74 hours of co-culture, and IFNγ levels were measured using human IFNγ ELISA Kit (R&D Systems) according to manufacturer instructions. The percentage of IFNγ secretion rescue was calculated using following formula: 100%−(IFNγ in samples treated with NECA and Compound A alone or combination with bintrafusp alfa minus IFNγ in control sample)+(IFNγ in samples with NECA minus IFNγ in control sample)*100%. Representative graph with the results shown as average of three samples per treatment group±SEM. P-values are calculated using one-way ANOVA Tukey comparison test.

FIG. 12: Compound A and bintrafusp alfa showed no combination effect in the CD73-KO 4T1 tumor model. Female BALB/c mice were inoculated with 1×10⁵ CD73 KO 4T1 tumor cells into the right mammary fat pad and were treated with Compound A (300 mg/kg po, bid), bintrafusp alfa (24.6 mg/kg iv, days 0, 3, 6), Compound A+bintrafusp alfa, when average tumor volume reached approximately 60 mm³. Vehicle (po, bid) and isotype control antibody injections (20 mg/kg iv, days 0, 3, 6) were used as controls. (A) Average tumor volumes with SEM, and (B) individual tumor volumes are presented. Tumor volume data were analyzed using two-way ANOVA followed by Tukey's multiple comparison test.

### DETAILED DESCRIPTION OF THE INVENTION

Each of the embodiments described herein can be combined with any other embodiment described herein not inconsistent with the embodiment with which it is combined. Furthermore, unless incompatible in a given context, wherever a compound is stipulated which is capable of ionization (e.g. protonation or deprotonation), the definition of said compound includes any pharmaceutically acceptable salts thereof. Accordingly, the phrase “or a pharmaceutically acceptable salt thereof” is implicit in the description of all compounds described herein. Embodiments within an aspect as described below can be combined with any other embodiments not inconsistent within the same aspect or a different aspect. For instance, embodiments of any of the treatment methods of the present invention can be combined with any embodiments of the combination products of the present invention or pharmaceutical composition of the present invention, and vice versa. Likewise, any detail or feature given for the treatment methods of the present invention apply—if not inconsistent—to those of the combination products of the present invention and pharmaceutical compositions of the present invention, and vice versa.

The present invention may be understood more readily by reference to the detailed description above and below of the particular and preferred embodiments of the invention and the examples included herein. It is to be understood that the terminology used herein is for the purpose of describing specific embodiments only and is not intended to be limiting. It is further to be understood that unless specifically defined herein, the terminology used herein is to be given its traditional meaning as known in the relevant art. So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

### Definitions

“A”, “an”, and “the” include plural referents unless the context clearly dictates otherwise. Thus, for example, reference to an antibody refers to one or more antibodies or at least one antibody. As such, the terms “a” (or “an”), “one or more”, and “at least one” are used interchangeably herein.

The term “about” when used to modify a numerically defined parameter refers to any minimal alteration in such parameter that does not change the overall effect, e.g., the efficacy of the agent in treatment of a disease or disorder. In some embodiments, the term “about” means that the parameter may vary by as much as 10% below or above the stated numerical value for that parameter.

An “adenosine inhibitor” is a molecule that inhibits adenosine-mediated signaling. Possible effects of such inhibition include the removal of immunosuppression in the tumor microenvironment and/or the removal of pro-tumorigenic effects of adenosine-mediated signaling. In some embodiments, the adenosine inhibitor binds to adenosine or an adenosine receptor to inhibit the interaction between these molecules. In some embodiments, the adenosine inhibitor inhibits the activity of one or more proteins selected from the group consisting of CD73, CD39, CD38, adenosine receptor A_{2A} and adenosine receptor A_{2B}.

A cancer that has “high adenosine-mediated signaling” refers to a cancer that has elevated levels of adenosine-mediated signaling, as compared to normal, non-cancerous tissue. Ways of measuring the activity of the adenosine signaling pathway are well-known to the person skilled in the art and include measuring the concentration of adenosine and/or CD73 in the tumor microenvironment, as well as measuring downstream effects of the pathway, such as the expression of adenosine-responsive genes. In some embodiments, the cancer having high adenosine-mediated signaling refers to an adenosine-rich cancer. In some embodiments, the adenosine-rich cancer has at least 0.5 μM, at least 0.75 μM, at least 1 μM, at least 1.5 μM, at least 2 μM, at least 5 μM, at least 10 μM, at least 15 μM, at least 20 μM or at least 25 μM adenosine in the tumor microenvironment. The adenosine concentration in the tumor microenvironment may, for instance, be measured in patient-derived xenografts (PDX). In such case, extracellular fluid from the PDX can be obtained by microdialysis and adenosine be quantified by LC-MS. The adenosine concentration may also be determined in accordance with the method described by Goodwin et al., Anal Biochem 2019; 568:78-88; Blay et al., Cancer Res. (1997) 57:2602-5 and Hatfield et al., J Mol Med. (2014) 92:1283-92 also describe methods for measuring extracellular adenosine levels in tumors. In some embodiments, the adenosine-rich cancer has adenosine levels in the tumor microenvironment that are high enough for the adenosine A_{2B} receptor to mediate signaling. In some embodiments, the cancer having high adenosine-mediated signaling refers to a cancer in which adenosine-mediated signaling exerts an immunosuppressive effect. In some embodiments, the cancer having high adenosine-mediated signaling refers to a cancer that is CD73 positive. In some embodiments, the cancer has high adenosine-mediated signaling as reflected by an adenosine gene-expression signature, which may be measured, for instance, in peripheral blood or tumor samples. In some embodiments, the adenosine gene-expression signature comprises evaluating the expression of CD73 and/or tissue non-specific alkaline phosphatase (TNAP). In some embodiments, the adenosine gene-expression signature comprises evaluating the expression of CXCL1, CXCL2, CXCL3, CXCL5, CXCL6, CXCL8, IL13 and PTGS2, which may be measured, for instance, in peripheral blood mononuclear cells (PBMC). In some embodiments, the adenosine gene-expression signature comprises evaluating the expression of one or more of PPARG, CYBB, COL3A1, FOXP3, LAG3, APP, CD81, GPI, PTGS2, CASP1, FOS, MAPK1, MAPK3, and CREB1. In some embodiments, the adenosine gene-expression signature comprises evaluating the expression of one or more enzymes of the adenosine signaling pathway, such as CD39, CD73, the adenosine A_{2A} receptor and the adenosine A_{2B} receptor.

An “adenosine receptor” is a G-protein-coupled receptor. There are four major subtypes of adenosine receptors—referred to as A₁, A_{2A}, A_{2B} and A₃. Though the same adenosine receptor can couple to different G-proteins, adenosine A₁ and A₃ receptors usually couple to inhibitory G-proteins referred to as Gᵢ and G₀ which inhibit adenylate cyclase and down-regulate cellular cAMP levels. In contrast, the adenosine A_{2A} and A_{2B} receptors couple to stimulatory G-proteins referred to as G_{S} that activate adenylate cyclase and increase intracellular levels of cAMP (Linden J., Annu. Rev. Pharmacol. Toxicol., 41: 775-87 2001).

An “adenosine receptor inhibitor” as used herein refers to a molecule that inhibits the activity of an adenosine receptor, e.g., by inhibiting the interaction between adenosine and an adenosine receptor. Possible effects of such inhibition include the removal of immunosuppression resulting from signaling on the adenosine signaling axis. Inhibition in this context need not be complete or 100%. Instead, inhibition means reducing, decreasing or abrogating binding between adenosine and the adenosine receptor and/or reducing, decreasing or abrogating adenosine-mediated signaling. In some embodiments, the adenosine receptor inhibitor is an adenosine A_{2A} and/or A_{2B} receptor inhibitor, i.e. it inhibits the activity of the adenosine A_{2A} and/or A_{2B} receptor. In some embodiments, the adenosine receptor inhibitor is an adenosine A_{2A} and A_{2B} receptor inhibitor. In some embodiments, the adenosine receptor inhibitor primarily or selectively inhibits the adenosine A_{2A} and/or A_{2B} receptor, i.e. its inhibitory activity on the adenosine A_{2A} and/or A_{2B} receptor is substantially higher than on other adenosine receptors. In some embodiments, the adenosine A_{2A} and/or A_{2B} receptor inhibitor shows at least 10-fold or at least 100-fold selectivity against other A₁, and A₃ adenosine receptors. In some embodiments, the adenosine A_{2A} and/or A_{2B} receptor inhibitor shows at least 50-fold selectivity against other A₁ adenosine receptors and at least 1000-fold selectivity against other A₃ adenosine receptors. In some embodiments, the adenosine A_{2A} and/or A_{2B} receptor inhibitor shows at least 100-fold selectivity against other A₁ adenosine receptors and at least 1000-fold selectivity against other A₃ adenosine receptors. The antagonistic activity of an adenosine receptor inhibitor on the adenosine A_{2A} receptor can be quantified, for instance, by an assay that measures interleukin-2 (IL-2) production by human T cells that gets suppressed by adenosine via the adenosine A_{2A} receptor. Specifically, human T cells are stimulated with anti-CD3/anti-CD28 coated Dynabeads, followed by treatment for 48 hours with a serial dilution of the adenosine receptor inhibitor in the presence of 10 μM of the adenosine analogue NECA. The antagonistic activity of the adenosine receptor inhibitor is then quantified by measuring with ELISA the rescue of IL-2 secretion from human T cells suppressed by 10 μM NECA. In some embodiments, the corresponding IC₅₀ of the adenosine receptor inhibitor in such IL-2 rescue assay is less than 1000 nm, less than 500 nm, less than 200 nm or less than 100 nm. The inhibitory activity of an adenosine receptor inhibitor on the adenosine A_{2B} receptor can be quantified, for instance, by an assay that measures the adenosine-dependent A_{2B}-driven VEGF production from human macrophages (Ryzhov, S. et al. Neoplasia, 2008; 10(9), 987-995; Ryzhov, S. et al. Mol Pharmacol, 2014; 85, 62-73; Sorrentino, C. et al. Oncotarget, 2015; 6(29), 27478-27489). Specifically, macrophages are stimulated with 10 ng/mL of LPS and treated for 48 hours with a serial dilution of the adenosine receptor inhibitor in the presence of 10 μM of the adenosine analogue NECA. The antagonistic activity of the adenosine receptor inhibitor is then quantified by measuring the VEGF concentration in the cell culture supernatant of the macrophages using ELISA. In some embodiments, the corresponding IC₅₀ of the adenosine receptor inhibitor in such VEGF inhibition assay is less than 1000 nm, less than 500 nm, less than 200 nm, less than 100 nm or less than 50 nm. In some embodiments, the adenosine A_{2A} and A_{2B} receptor inhibitor has a corresponding IC₅₀ for inhibiting the adenosine A_{2A} receptor activity in such IL-2 rescue assay of less than 500 nm and a corresponding IC₅₀ for inhibiting the adenosine A_{2B} receptor activity in such VEGF inhibition assay of less than 500 nm. In some embodiments, the adenosine A_{2A} and A_{2B} receptor inhibitor has a corresponding IC₅₀ for inhibiting the adenosine A_{2A} receptor activity in such IL-2 rescue assay of less than 100 nm and a corresponding IC₅₀ for inhibiting the adenosine A_{2B} receptor activity in such VEGF inhibition assay of less than 50 nm. The inhibitory activity of an adenosine receptor inhibitor on the adenosine A_{2A} receptor can also be quantified by an assay that measures the phosphorylation of CREB in human whole blood CD8+ T cells that gets promoted by adenosine via the adenosine A_{2A} receptor (Sassone-Corsi, P. Cold Spring Harb Perspect Biol, 2012; 4, a011148). Specifically, human whole blood from six independent donors is incubated for 15 minutes with serial dilutions of the adenosine receptor inhibitor followed by stimulation with 10 μM NECA for 45 minutes, and subsequent analysis of pCREB signal within CD8+ T cells by flow cytometry. In some embodiments, the corresponding IC₅₀ of the adenosine receptor inhibitor in such pCREB inhibition assay is less than 1000 nm, less than 500 nm or less than 200 nm.

“Administering” or “administration of” a drug to a patient (and grammatical equivalents of this phrase) refers to direct administration, which may be administration to a patient by a medical professional or may be self-administration, and/or indirect administration, which may be the act of prescribing a drug, e.g., a physician who instructs a patient to self-administer a drug or provides a patient with a prescription for a drug is administering the drug to the patient.

An “amino acid difference” refers to a substitution, a deletion or an insertion of an amino acid.

“Antibody” is an immunoglobulin (Ig) molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact polyclonal or monoclonal antibodies, but also, unless otherwise specified, any antigen-binding fragment or antibody fragment thereof that competes with the intact antibody for specific binding, as well as any protein comprising such antigen-binding fragment or antibody fragment thereof, including fusion proteins (e.g., antibody-drug conjugates, an antibody fused to a cytokine or an antibody fused to a cytokine receptor), antibody compositions with poly-epitopic specificity, and multi-specific antibodies (e.g., bispecific antibodies). The basic 4-chain antibody unit is a heterotetrameric glycoprotein composed of two identical light (L) chains and two identical heavy (H) chains. An IgM antibody consists of 5 of the basic heterotetramer units along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 Daltons. Each L chain is linked to an H chain by one covalent disulfide bond, while the two H chains are linked to each other by one or more disulfide bonds depending on the H chain isotype. Each H and L chain also has regularly spaced intra-chain disulfide bridges. Each H chain has, at the N-terminus, a variable domain (V_{H}) followed by three constant domains (C_{H}) for each of the α and γ chains and four C_{H} domains for p and E isotypes. Each L chain has at the N-terminus, a variable domain (V_{L}) followed by a constant domain at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see e.g., Basic and Clinical Immunology, 8^{th} Edition, Sties et al. (eds.), Appleton & Lange, Norwalk, C T, 1994, page 71 and Chapter 6. The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins: IgA, IgD, IgE, IgG and IgM, having heavy chains designated α, δ, ε, γ and μ, respectively. The γ and α classes are further divided into subclasses on the basis of relatively minor differences in the C_{H} sequence and function, e.g., humans express the following subclasses: IgG1, IgG2A, IgG2B, IgG3, IgG4, IgA1, and IgK1.

“Antigen-binding fragment” of an antibody or “antibody fragment” comprises a portion of an intact antibody, which is still capable of antigen binding. Antigen-binding fragments include, for example, Fab, Fab′, F(ab′)₂, Fd, and Fv fragments, domain antibodies (dAbs, e.g., shark and camelid antibodies), fragments including CDRs, single chain variable fragment antibodies (scFv), single-chain antibody molecules, multi-specific antibodies formed from antibody fragments, maxibodies, nanobodies, minibodies, intrabodies, diabodies, triabodies, tetrabodies, v-NAR and bis-scFv, linear antibodies (see e.g., U.S. Pat. No. 5,641,870, Example 2; Zapata et al. (1995) Protein Eng. 8HO: 1057), and polypeptides that contain at least a portion of an immunoglobulin that is sufficient to confer specific antigen binding to the polypeptide. Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment, which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments were originally produced as pairs of Fab′ fragments which have hinge cysteines between them. Other chemical couplings of antibody fragments are also known.

“Anti-PD-L1 antibody” or “anti-PD-1 antibody” means an antibody, or an antigen-binding fragment thereof, that specifically binds to PD-L1 or PD-1 respectively and blocks binding of PD-L1 to PD-1. In any of the treatment methods, medicaments and uses of the present invention in which a human subject is being treated, the anti-PD-L1 antibody specifically binds to human PD-L1 and blocks binding of human PD-L1 to human PD-1. In any of the treatment methods, medicaments and uses of the present invention in which a human subject is being treated, the anti-PD-1 antibody specifically binds to human PD-1 and blocks binding of human PD-L1 to human PD-1. The antibody may be a monoclonal antibody, human antibody, humanized antibody or chimeric antibody, and may include a human constant region. In some embodiments the human constant region is selected from the group consisting of IgG1, IgG2, IgG3 and IgG4 constant regions, and in some embodiments, the human constant region is an IgG1 or IgG4 constant region. In some embodiments, the antigen-binding fragment is selected from the group consisting of Fab, Fab′-SH, F(ab′)2, scFv and Fv fragments.

“Anti-PD(L)1 antibody” refers to an anti-PD-L1 antibody or an anti-PD-1 antibody.

“Bintrafusp alfa”, also known as M7824, is well understood in the art. Bintrafusp alfa is an anti-PD-L1:TGFβRII fusion protein and described under the CAS Registry Number 1918149-01-5. It is also described in WO 2015/118175 and further elaborated in Lan et al (Lan et al, Sci. Transl. Med. 10, 2018, p. 1-15). In particular, bintrafusp alfa is a fully human IgG1 monoclonal antibody against human PD-L1 fused to the extracellular domain of human TGF-β receptor II (TGFβRII). As such, bintrafusp alfa is a bifunctional fusion protein that simultaneously blocks PD-L1 and TGF-β pathways. In particular, WO 2015/118175 describes bintrafusp alfa on page 34 in Example 1 thereof as follows (bintrafusp alfa is referred to in this passage as “anti-PD-L1/TGFβ Trap”): “Anti-PD-L1/TGFβ Trap is an anti-PD-L1 antibody-TGFβ Receptor II fusion protein. The light chain of the molecule is identical to the light chain of the anti-PD-L1 antibody (SEQ ID NO: 1). The heavy chain of the molecule (SEQ ID NO:3) is a fusion protein comprising the heavy chain of the anti-PD-L1 antibody (SEQ ID NO: 2) genetically fused to via a flexible (Gly4Ser)4Gly linker (SEQ ID NO:11) to the N-terminus of the soluble TGFβ Receptor II (SEQ ID NO: 10). At the fusion junction, the C-terminal lysine residue of the antibody heavy chain was mutated to alanine to reduce proteolytic cleavage.”

“Biomarker” generally refers to biological molecules, and quantitative and qualitative measurements of the same, that are indicative of a disease state. “Prognostic biomarkers” correlate with disease outcome, independent of therapy. For example, tumor hypoxia is a negative prognostic marker—the higher the tumor hypoxia, the higher the likelihood that the outcome of the disease will be negative. “Predictive biomarkers” indicate whether a patient is likely to respond positively to a particular therapy, e.g., HER2 profiling is commonly used in breast cancer patients to determine if those patients are likely to respond to Herceptin (trastuzumab, Genentech). “Response biomarkers” provide a measure of the response to a therapy and so provide an indication of whether a therapy is working. For example, decreasing levels of prostate-specific antigen generally indicate that anti-cancer therapy for a prostate cancer patient is working. When a marker is used as a basis for identifying or selecting a patient for a treatment described herein, the marker can be measured before and/or during treatment, and the values obtained are used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits; or (h) toxicity. As would be well understood by one in the art, measurement of a biomarker in a clinical setting is a clear indication that this parameter was used as a basis for initiating, continuing, adjusting and/or ceasing administration of the treatments described herein.

By “cancer” is meant a collection of cells multiplying in an abnormal manner. As used herein, the term “cancer” refers to all types of cancer, neoplasm, malignant or benign tumors found in mammals, including leukemia, carcinomas, and sarcomas. Exemplary cancers include breast cancer, ovarian cancer, colon cancer, liver cancer, kidney cancer, lung cancer, pancreatic cancer, glioblastoma. Additional examples include cancer of the brain, lung cancer, non-small cell lung cancer, melanoma, sarcomas, prostate cancer, cervix cancer, stomach cancer, head and neck cancers, uterus cancer, mesothelioma, metastatic bone cancer, medulloblastoma, Hodgkin's Disease, Non-Hodgkin's Lymphoma, multiple myeloma, neuroblastoma, rhabdomyosarcoma, primary thrombocytosis, primary macrobulinemia, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, and neoplasms of the endocrine and exocrine pancreas.

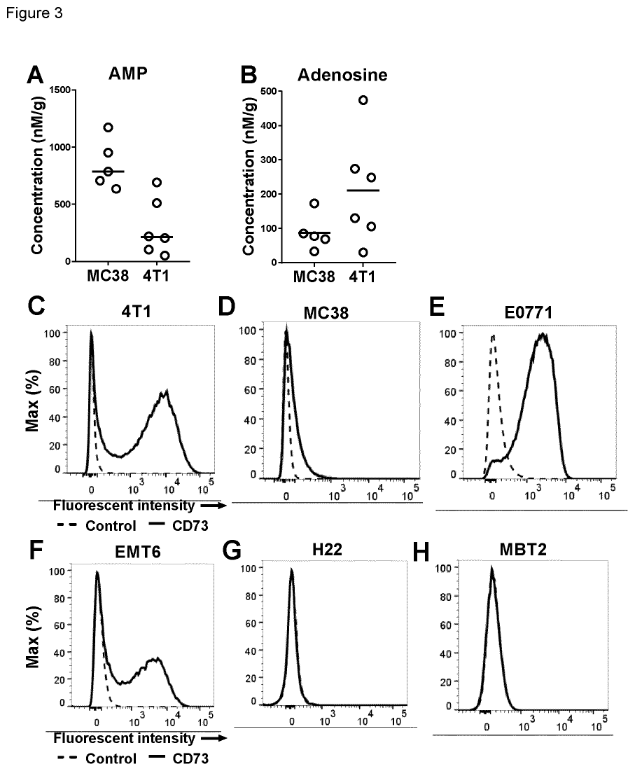

A “CD73 positive” cancer is one comprising cells in the tumor microenvironment, which have CD73 present at their cell surface, and/or one producing sufficient levels of CD73 at the surface of cells thereof, such that the levels of adenosine are elevated in the tumor microenvironment as compared to normal, non-cancerous tissue. Methods for detecting CD73 expression are described below. In some embodiments, at least 1%, at least 5%, at least 10%, at least 25%, at least 50% or at least 75% of the cells in the tumor microenvironment have CD73 present on their cell surface. In some embodiments, at least 1%, at least 5%, at least 10%, at least 25%, at least 50% or at least 75% of the tumor cells have CD73 present on their cell surface. In some embodiments, the CD73 positive tumor is a tumor for which a separate peak, which is shifted to the right as compared to the corresponding healthy tissue, is observed in a FACS plot when a sample of the tumor is analyzed using a fluorescently-labelled anti-CD73 antibody. Exemplary FACS plots where such separate peak is observable are shown in FIG. 3 (see the separate peak observed for the 4T1, EMT6 and E0771 samples, as compared to the MC38, H22 and MBT2 samples). In some embodiments, CD73 expression is assessed as copy number of CD73 protein per cell. In some embodiments, the CD73 positive tumor is a tumor with an increased copy number of CD73 protein per cell as compared to the corresponding healthy tissue. One way of quantifying the copy number of CD73 protein per cell is as follows: tumor samples are stained with viability dye and anti-CD73 antibody, allowing for assessment of CD73 expression on viable cells using flow cytometry. At the same time, beads from the Quantum™ Simply Cellular® kit are labeled to saturation using the same anti-CD73 antibody. The kit contains five bead populations, one blank and four with increasing levels of Fc-specific capture antibody. The beads and cells are analyzed by flow cytometry on the same day using the same settings. The fluorescence channel values associated with each bead populations' antibody binding capacity (ABC) are used to calculate a standard curve. ABC values are then assigned to the stained cell samples using the standard curve. Presuming monovalent antibody-to-receptor binding, the assigned ABC value equals the number of surface CD73 copy numbers per cell in a given sample. In some embodiments, the number of CD73 proteins per cell in a CD73 positive cancer is at least 1000, at least 5000, at least 10 000, at least 20 000 or at least 40 000. In some embodiments, the CD73 positive cancer is a cancer with a CD73 expression that is at least as high as the CD73 expression of one the cell lines selected from the group consisting of E0771 (ATCC CRL-3461), EMT6 (ATCC CRL-2755) and 4T1 (ATCC CRL-2539).

“CDRs” are the complementarity determining region amino acid sequences of an antibody, antibody fragment or antigen-binding fragment. These are the hypervariable regions of immunoglobulin heavy and light chains. There are three heavy chain and three light chain CDRs (or CDR regions) in the variable portion of an immunoglobulin.

“Clinical outcome”, “clinical parameter”, “clinical response”, or “clinical endpoint” refers to any clinical observation or measurement relating to a patient's reaction to a therapy. Non-limiting examples of clinical outcomes include tumor response (TR), overall survival (OS), progression free survival (PFS), disease free survival, time to tumor recurrence (TTR), time to tumor progression (TTP), relative risk (RR), toxicity, or side effect.

“Combination” as used herein refers to the provision of a first active modality in addition to one or more further active modalities (wherein one or more active modalities may be fused). Contemplated within the scope of the combinations described herein, are any regimen of combination modalities or partners (i.e., active compounds, components or agents), such as a combination of a PD-1 inhibitor, a TGFβ inhibitor and an adenosine receptor inhibitor, encompassed in single or multiple compounds and compositions. It is understood that any modalities within a single composition, formulation or unit dosage form (i.e., a fixed-dose combination) must have the identical dose regimen and route of delivery. It is not intended to imply that the modalities must be formulated for delivery together (e.g., in the same composition, formulation or unit dosage form). The combined modalities can be manufactured and/or formulated by the same or different manufacturers. The combination partners may thus be, e.g., entirely separate pharmaceutical dosage forms or pharmaceutical compositions that are also sold independently of each other. In some embodiments, the TGFβ inhibitor is fused to the PD-1 inhibitor and therefore encompassed within a single composition and having an identical dose regimen and route of delivery.

“Combination therapy”, “in combination with” or “in conjunction with” as used herein denotes any form of concurrent, parallel, simultaneous, sequential or intermittent treatment with at least two distinct treatment modalities (i.e., compounds, components, targeted agents or therapeutic agents). As such, the terms refer to administration of one treatment modality before, during, or after administration of the other treatment modality to the subject. The modalities in combination can be administered in any order. The therapeutically active modalities are administered together (e.g., simultaneously in the same or separate compositions, formulations or unit dosage forms) or separately (e.g., on the same day or on different days and in any order as according to an appropriate dosing protocol for the separate compositions, formulations or unit dosage forms) in a manner and dosing regimen prescribed by a medical care taker or according to a regulatory agency. In general, each treatment modality will be administered at a dose and/or on a time schedule determined for that treatment modality. Optionally, four or more modalities may be used in a combination therapy. Additionally, the combination therapies provided herein may be used in conjunction with other types of treatment. For example, other anti-cancer treatment may be selected from the group consisting of chemotherapy, surgery, radiotherapy (radiation) and/or hormone therapy, amongst other treatments associated with the current standard of care for the subject.

“Complete response” or “complete remission” refers to the disappearance of all signs of cancer in response to treatment. This does not always mean the cancer has been cured.

“Comprising”, as used herein, is intended to mean that the compositions and methods include the recited elements, but not excluding others. “Consisting essentially of”, when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this invention. Accordingly, it is intended that the methods and compositions can include additional steps and components (comprising) or alternatively including steps and compositions of no significance (consisting essentially of) or alternatively, intending only the stated method steps or compositions (consisting of).

“Dose” and “dosage” refer to a specific amount of active or therapeutic agents for administration. Such amounts are included in a “dosage form,” which refers to physically discrete units suitable as unitary dosages for human subjects and other mammals, each unit containing a predetermined quantity of active agent calculated to produce the desired onset, tolerability, and therapeutic effects, in association with one or more suitable pharmaceutical excipients such as carriers.

“Fc” is a fragment comprising the carboxy-terminal portions of both H chains held together by disulfides. The effector functions of antibodies are determined by sequences in the Fc region, the region which is also recognized by Fc receptors (FcR) found on certain types of cells.

The term “fusion molecule” is well understood in the art and it will be appreciated that the molecule comprising a fused PD-1 inhibitor and TGFβ inhibitor as referred to herein includes an Ig:TGFβR fusion protein, such as an anti-PD-1:TGFβR fusion protein or an anti-PD-L1:TGFβR fusion protein. An Ig:TGFβR fusion protein is an antibody (in some embodiments, a monoclonal antibody, e.g., in homodimeric form) or an antigen-binding fragment thereof fused to a TGF-β receptor. The nomenclature anti-PD-L1:TGFβRII fusion protein indicates an anti-PD-L1 antibody, or an antigen-binding fragment thereof, fused to a TGF-β receptor II or a fragment of the extracellular domain thereof that is capable of binding TGF-β. The nomenclature anti-PD-1:TGFβRII fusion protein indicates an anti-PD-1 antibody, or an antigen-binding fragment thereof, fused to a TGF-β receptor II or a fragment of the extracellular domain thereof that is capable of binding TGF-β. The nomenclature anti-PD(L)1:TGFβRII fusion protein, indicates an anti-PD-1 antibody or an antigen-binding fragment thereof, or an anti-PD-L1 antibody or an antigen-binding fragment thereof, fused to a TGF-β receptor II or a fragment of the extracellular domain thereof that is capable of binding TGF-β.

“Fv” is the minimum antibody fragment, which contains a complete antigen-recognition and antigen-binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen-binding specificity to the antibody. However, even a single variable domain (or half of an Fv comprising only three HVRs specific for an antigen) has the ability to recognize and bind antigen, although at a lower affinity than the entire binding site.

“Human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries (see e.g., Hoogenboom and Winter (1991), JMB 227: 381; Marks et al. (1991) JMB 222: 581). Also available for the preparation of human monoclonal antibodies are methods described in Cole et al. (1985) Monoclonal Antibodies and Cancer Therapy, Alan R. Liss, page 77; Boerner et al. (1991), J. Immunol 147(l): 86; van Dijk and van de Winkel (2001) Curr. Opin. Pharmacol 5: 368). Human antibodies can be prepared by administering the antigen to a transgenic animal that has been modified to produce such antibodies in response to antigenic challenge but whose endogenous loci have been disabled, e.g., immunized xenomice (see e.g., U.S. Pat. Nos. 6,075,181; and 6,150,584 regarding XENOMOUSE technology). See also, for example, Li et al. (2006) PNAS USA, 103: 3557, regarding human antibodies generated via a human B-cell hybridoma technology.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from an HVR of the recipient are replaced by residues from an HVR of a non-human species (donor antibody) such as mouse, rat, rabbit, or non-human primate having the desired specificity, affinity and/or capacity. In some instances, framework (“FR”) residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. The number of these amino acid substitutions in the FR are typically no more than 6 in the H chain, and no more than 3 in the L chain. The humanized antibody optionally will also comprise at least a portion of an immunoglobulin constant region (Fc), typically that of a human immunoglobulin. For further details, see e.g., Jones et al. (1986) Nature 321: 522; Riechmann et al. (1988), Nature 332: 323; Presta (1992) Curr. Op. Struct. Biol. 2: 593; Vaswani and Hamilton (1998), Ann. Allergy, Asthma & Immunol. 1: 105; Harris (1995) Biochem. Soc. Transactions 23: 1035; Hurle and Gross (1994) Curr. Op. Biotech. 5: 428; and U.S. Pat. Nos. 6,982,321 and 7,087,409.

“Infusion” or “infusing” refers to the introduction of a drug-containing solution into the body through a vein for therapeutic purposes. Generally, this is achieved via an intravenous (IV) bag.

“Metastatic” cancer refers to cancer which has spread from one part of the body (e.g., the lung) to another part of the body.

“Monoclonal antibody”, as used herein, refers to an antibody obtained from a population of substantially homogeneous antibodies, i.e., the individual antibodies comprising the population are identical except for possible naturally occurring mutations and/or post-translation modifications (e.g., isomerizations and amidations) that may be present in minor amounts. Monoclonal antibodies are highly specific, being directed against a single antigenic site. In contrast to polyclonal antibody preparations, which typically include different antibodies directed against different determinants (epitopes), each monoclonal antibody is directed against a single determinant on the antigen. In addition to their specificity, the monoclonal antibodies are advantageous in that they are synthesized by the hybridoma culture and uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein (1975) Nature 256: 495; Hongo et al. (1995) Hybridoma 14 (3): 253; Harlow et al. (1988) Antibodies: A Laboratory Manual (Cold Spring Harbor Laboratory Press, 2^{nd} ed.; Hammerling et al. (1981) In: Monoclonal Antibodies and T-Cell Hybridomas 563 (Elsevier, N.Y.), recombinant DNA methods (see e.g., U.S. Pat. No. 4,816,567), phage-display technologies (see e.g., Clackson et al. (1991) Nature 352: 624; Marks et al. (1992) JMB 222: 581; Sidhu et al. (2004) JMB 338(2): 299; Lee et al. (2004) JMB 340(5): 1073; Fellouse (2004) PNAS USA 101(34): 12467; and Lee et al. (2004) J. Immunol. Methods 284(1-2): 119), and technologies for producing human or human-like antibodies in animals that have parts or all of the human immunoglobulin loci or genes encoding human immunoglobulin sequences (see e.g., WO 1998/24893; WO 1996/34096; WO 1996/33735; WO 1991/10741; Jakobovits et al. (1993) PNAS USA 90: 2551; Jakobovits et al. (1993) Nature 362: 255; Bruggemann et al. (1993) Year in Immunol. 7: 33; U.S. Pat. Nos. 5,545,807; 5,545,806; 5,569,825; 5,625,126; 5,633,425; and 5,661,016; Marks et al. (1992) Bio/Technology 10: 779; Lonberg et al. (1994) Nature 368: 856; Morrison (1994) Nature 368: 812; Fishwild et al. (1996) Nature Biotechnol. 14: 845; Neuberger (1996), Nature Biotechnol. 14: 826; and Lonberg and Huszar (1995), Intern. Rev. Immunol. 13: 65-93). The monoclonal antibodies herein specifically include chimeric antibodies (immunoglobulins) in which a portion of the heavy and/or light chain is identical to or homologous to corresponding sequences in antibodies derived from a particular species or belonging to a particular antibody class or subclass, while the remainder of the chain(s) is (are) identical to or homologous to corresponding sequences in antibodies derived from another species or belonging to another antibody class or subclass, as well as fragments of such antibodies, so long as they exhibit the desired biological activity (see e.g., U.S. Pat. No. 4,816,567; Morrison et al. (1984) PNAS USA, 81: 6851).

“Objective response” refers to a measurable response, including complete response (CR) or partial response (PR).

“Partial response” refers to a decrease in the size of one or more tumors or lesions, or in the extent of cancer in the body, in response to treatment.

“Patient” and “subject” are used interchangeably herein to refer to a mammal in need of treatment for a cancer. Generally, the patient is a human diagnosed or at risk for suffering from one or more symptoms of a cancer. In certain embodiments a “patient” or “subject” may refer to a non-human mammal, such as a non-human primate, a dog, cat, rabbit, pig, mouse, or rat, or animals used, e.g., in screening, characterizing, and evaluating drugs and therapies.

“PD-1 inhibitor” as used herein refers to a molecule that inhibits the PD-1 pathway, e.g., by inhibiting the interaction of PD-1 axis binding partners, such as between the PD-1 receptor and the PD-L1 and/or PD-L2 ligand. Possible effects of such inhibition include the removal of immunosuppression resulting from signaling on the PD-1 signaling axis. Inhibition in this context need not be complete or 100%. Instead, inhibition means reducing, decreasing or abrogating binding between PD-1 and one or more of its ligands and/or reducing, decreasing or abrogating signaling through the PD-1 receptor. In some embodiments, the PD-1 inhibitor binds to PD-L1 or PD-1 to inhibit the interaction between these molecules, such as an anti-PD-1 antibody or an anti-PD-L1 antibody. In some embodiments, the PD-1 inhibitor is a PD-L1 antibody and such antibody may be fused to the TGFβ inhibitor, e.g., as an anti-PD-L1:TGFβRII fusion protein.

“PD-L1 expression” as used herein means any detectable level of expression of PD-L1 protein on the cell surface or of PD-L1 mRNA within a cell or tissue. PD-L1 protein expression may be detected with a diagnostic PD-L1 antibody in an IHC assay of a tumor tissue section or by flow cytometry. Alternatively, PD-L1 protein expression by tumor cells may be detected by PET imaging, using a binding agent (e.g., antibody fragment, affibody and the like) that specifically binds to PD-L1. Techniques for detecting and measuring PD-L1 mRNA expression include RT-PCR and real-time quantitative RT-PCR.

A “PD-L1 positive” or “PD-L1 high” cancer is one comprising cells, which have PD-L1 present at their cell surface, and/or one producing sufficient levels of PD-L1 at the surface of cells thereof, such that an anti-PD-L1 antibody has a therapeutic effect, mediated by the binding of the said anti-PD-L1 antibody to PD-L1. Methods of detecting a biomarker, such as PD-L1 or CD73 for example, on a cancer or tumor, are routine in the art and are contemplated herein. Non-limiting examples include immunohistochemistry (IHC), immunofluorescence and fluorescence activated cell sorting (FACS). Several approaches have been described for quantifying PD-L1 protein expression in IHC assays of tumor tissue sections. The ratio of PD-L1 positive cells is oftentimes expressed as a Tumor Proportion Score (TPS) or a Combined Positive Score (CPS). The TPS describes the percentage of viable tumor cells with partial or complete membrane staining (e.g., staining for PD-L1). The CPS is the number of PD-L1 staining cells (tumor cells, lymphocytes, macrophages) divided by the total number of viable tumor cells, multiplied by 100. For instance, in some embodiments, “PD-L1 high” refers to 80% PD-L1 positive tumor cells as determined by the PD-L1 Dako IHC 73-10 assay, or tumor proportion score (TPS) 50% as determined by the Dako IHC 22C3 PharmDx assay. Both IHC 73-10 and IHC 22C3 assays select a similar patient population at their respective cutoffs. In certain embodiments, Ventana PD-L1 (SP263) assay, which has high concordance with 22C3 PharmDx assay (see Sughayer et al., Appl. Immunohistochem. Mol. Morphol., 27:663-666 (2019)), can also be used for determining the PD-L1 expression level. Another assay for determining PD-L1 expression in cancers is the Ventana PD-L1 (SP142) assay. In some embodiments, a cancer is counted as PD-L1 or CD73 positive if at least 1%, at least 5%, at least 25%, at least 50%, at least 75% or at least 80% of the tumor cells show PD-L1 or CD73 expression, respectively.

“Percent (%) sequence identity” with respect to a peptide or polypeptide sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2 or ALIGN software. Those skilled in the art can determine appropriate parameters for measuring alignment, including any algorithms needed to achieve maximal alignment over the full length of the sequences being compared.

“Pharmaceutically acceptable” indicates that the substance or composition must be compatible chemically and/or toxicologically, with the other ingredients comprising a formulation, and/or the mammal being treated therewith. “Pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof.

“Prodrug” refers to derivatives of the compounds of the present invention which have been modified by means of, for example, alkyl or acyl groups (see also amino- and hydroxyl-protecting groups below), sugars or oligopeptides and which are rapidly cleaved or liberated in the organism to form the effective molecules. These also include biodegradable polymer derivatives of the compound of the present invention, as described, for example, in Int. J. Pharm. 115 (1995), 61-67.

“Recurrent” cancer is one which has regrown, either at the initial site or at a distant site, after a response to initial therapy, such as surgery. A locally “recurrent” cancer is cancer that returns after treatment in the same place as a previously treated cancer.

“Reduction” of a symptom or symptoms (and grammatical equivalents of this phrase) refers to decreasing the severity or frequency of the symptom(s), or elimination of the symptom(s).

“Single-chain Fv”, also abbreviated as “sFv” or “scFv”, are antibody fragments that comprise the V_{H} and V_{L} antibody domains connected into a single polypeptide chain. In some embodiments, the sFv polypeptide further comprises a polypeptide linker between the V_{H} and V_{L} domains which enables the sFv to form the desired structure for antigen binding. For a review of the sFv, see e.g., Pluckthun (1994), In: The Pharmacology of Monoclonal Antibodies, vol. 113, Rosenburg and Moore (eds.), Springer-Verlag, New York, pp. 269.

“Solvates” refer to adductions of inert solvent molecules onto the compounds of the invention which form owing to their mutual attractive force. Solvates are, for example, hydrates, such as monohydrates or dihydrates, or alcoholates, i.e. addition compounds with alcohols, such as, for example, with methanol or ethanol.

By “substantially identical” is meant (1) a query amino acid sequence exhibiting at least 75%, 85%, 90%, 95%, 99% or 100% amino acid sequence identity to a subject amino acid sequence or (2) a query amino acid sequence that differs in not more than 20%, 30%, 20%, 10%, 5%, 1% or 0% of its amino acid positions from the amino acid sequence of a subject amino acid sequence and wherein a difference in an amino acid position is any of a substitution, deletion or insertion of an amino acid.

“Systemic” treatment is a treatment, in which the drug substance travels through the bloodstream, reaching and affecting cells all over the body.

“TGFβ inhibitor” as used herein refers to a molecule that inhibits the TGFβ pathway, e.g., by inhibiting the interaction between a TGFβ and a TGFβ receptor (TGFβR). Possible effects of such inhibition include the removal of immunosuppression resulting from signaling on the TGFβ signaling axis. Inhibition in this context need not be complete or 100%. Instead, inhibition means reducing, decreasing or abrogating binding between TGF-β and the TGFβR and/or reducing, decreasing or abrogating signaling through the TGFβR. In some embodiments, the TGFβ inhibitor binds to TGFβ or a TGFβR to inhibit the interaction between these molecules. In some embodiments, the TGFβ inhibitor comprises the extracellular domain of a TGFβRII, or a fragment of TGFβRII capable of binding TGFβ. In some embodiments, such TGFβ inhibitor is fused to the PD-1 inhibitor, e.g., as an anti-PD(L)1:TGFβRII fusion protein.

The term “TGF-β receptor” (TGFβR), as well as “TGF-β receptor I” (abbreviated as TGFβRI or TGFβR1) or “TGF-β receptor II” (abbreviated as TGFβRII or TGFβR2), are well known in the art. For the purposes of this disclosure, reference to such receptor includes the full receptor and fragments that are capable of binding TGF-β. In some embodiments, it is the extracellular domain of the receptor or a fragment of the extracellular domain that is capable of binding TGF-β. In some embodiments, the fragment of TGFβRII is selected from the group consisting of SEQ ID NO: 11, SEQ ID NO: 12 and SEQ ID NO: 13.

“Therapeutically effective amount” of a PD-1 inhibitor, a TGFβ inhibitor, or a VEGF inhibitor, in each case of the invention, refers to an amount effective, at dosages and for periods of time necessary, that, when administered to a patient with a cancer, will have the intended therapeutic effect, e.g., alleviation, amelioration, palliation, or elimination of one or more manifestations of the cancer in the patient, or any other clinical result in the course of treating a cancer patient. A therapeutic effect does not necessarily occur by administration of one dose, and may occur only after administration of a series of doses. Thus, a therapeutically effective amount may be administered in one or more administrations. Such therapeutically effective amount may vary according to factors such as the disease state, age, sex, and weight of the individual, and the ability of a PD-1 inhibitor, a TGFβ inhibitor, or a VEGF inhibitor to elicit a desired response in the individual. A therapeutically effective amount is also one in which any toxic or detrimental effects of a PD-1 inhibitor, a TGFβ inhibitor, or a VEGF inhibitor are outweighed by the therapeutically beneficial effects.

“Treating” or “treatment of” a condition or patient refers to taking steps to obtain beneficial or desired results, including clinical results. For purposes of this invention, beneficial or desired clinical results include, but are not limited to, alleviation, amelioration of one or more symptoms of a cancer; diminishment of extent of disease; delay or slowing of disease progression; amelioration, palliation, or stabilization of the disease state; or other beneficial results. It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

“Unit dosage form” as used herein refers to a physically discrete unit of therapeutic formulation appropriate for the subject to be treated. It will be understood, however, that the total daily usage of the compositions of the present invention will be decided by the attending physician within the scope of sound medical judgment. The specific effective dose level for any particular subject or organism will depend upon a variety of factors including the disorder being treated and the severity of the disorder; activity of specific active agent employed; specific composition employed; age, body weight, general health, sex and diet of the subject; time of administration, and rate of excretion of the specific active agent employed; duration of the treatment; drugs and/or additional therapies used in combination or coincidental with specific compound(s) employed, and like factors well known in the medical arts.

“Variable region” or “variable domain” of an antibody refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites.

As used herein, a plurality of items, structural elements, compositional elements, and/or materials may be presented in a common list for convenience. However, these lists should be construed as though each member of the list is individually identified as a separate and unique member.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

### DESCRIPTIVE EMBODIMENTS

### Therapeutic Combination and Method of Use Thereof

The present invention arose in part from the surprising discovery of a combination benefit for a PD-1 inhibitor, a TGFβ inhibitor, and an adenosine inhibitor. Treatment schedule and doses were designed to reveal potential synergies. Pre-clinical data demonstrated a synergy of the adenosine inhibitor when combined with the PD-1 inhibitor and the TGFβ inhibitor.

Thus, in one aspect, the present invention provides a PD-1 inhibitor, a TGFβ inhibitor, and an adenosine inhibitor, such as an adenosine A_{2A} and/or A_{2B} receptor inhibitor, for use in a method for treating a cancer in a subject comprising administering the PD-1 inhibitor, TGFβ inhibitor, and adenosine inhibitor to the subject, as well as a method for treating a cancer in a subject comprising administering a PD-1 inhibitor, a TGFβ inhibitor, and an adenosine inhibitor, such as an adenosine A_{2A} and/or A_{2B} receptor inhibitor, to the subject, as well as the use of a PD-1 inhibitor, a TGFβ inhibitor, and an adenosine inhibitor, such as an adenosine A_{2A} and/or A_{2B} receptor inhibitor, in the manufacture of a medicament for treating a cancer in a subject comprising administering the PD-1 inhibitor, TGFβ inhibitor, and adenosine inhibitor to the subject. It shall be understood that a therapeutically effective amount of the PD-1 inhibitor, TGFβ inhibitor, and adenosine inhibitor is applied in each method of treatment. In some embodiments, the PD-1 inhibitor is an anti-PD(L)1 antibody and the TGFβ inhibitor is a TGFβRII or an anti-TGFβ antibody. In some embodiments, the PD-1 inhibitor is fused to the TGFβ inhibitor. For instance, the PD-1 inhibitor and TGFβ inhibitor may be comprised in an anti-PD(L)1:TGFβRII fusion protein, such as an anti-PD-L1:TGFβRII fusion protein or an anti-PD-1:TGFβRII fusion protein. In some embodiments, the fusion molecule is an anti-PD-L1:TGFβRII fusion protein, e.g., an anti-PD-L1:TGFβRII fusion protein wherein the light chain sequences and the heavy chain sequences correspond to SEQ ID NO: 7 and SEQ ID NO: 8, respectively. In some embodiments, the adenosine inhibitor is an adenosine A_{2A} and/or A_{2B} receptor inhibitor. In some embodiments, the adenosine inhibitor is an adenosine A_{2A} and A_{2B} receptor inhibitor. In some embodiments, the adenosine inhibitor is (S)-7-Oxa-2-aza-spiro[4.5]decane-2-carboxylic acid [7-(3,6-dihydro-2H-pyran-4-yl)-4-methoxy-thiazolo[4,5-c]pyridin-2-yl]-amide.

The PD-1 inhibitor may inhibit the interaction between PD-1 and at least one of its ligands, such as PD-L1 or PD-L2, and thereby inhibit the PD-1 pathway, e.g., the immunosuppressive signal of PD-1. The PD-1 inhibitor may bind to PD-1 or one of its ligands, such as PD-L1. In one embodiment, the PD-1 inhibitor inhibits the interaction between PD-1 and PD-L1. In some embodiments, the PD-1 inhibitor is an anti-PD(L)1 antibody, such as an anti-PD-1 antibody or an anti-PD-L1 antibody, capable of inhibiting the interaction between PD-1 and PD-L1. In some embodiments, the anti-PD-1 antibody or anti-PD-L1 antibody is selected from the group consisting of pembrolizumab, nivolumab, avelumab, atezolizumab, durvalumab, spartalizumab, camrelizumab, sintilimab, tislelizumab, toripalimab, cemiplimab, and an antibody wherein the light chain sequences and the heavy chain sequences of the antibody correspond to SEQ ID NO: 7 and SEQ ID NO: 16, or to SEQ ID NO: 15 and SEQ ID NO: 14, respectively, or an antibody that competes for binding with any of the antibodies of this group. In some embodiments, the anti-PD-1 antibody or anti-PD-L1 antibody is one that is still capable of binding to PD-1 or PD-L1 and which amino acid sequence is substantially identical, e.g., has at least 90% sequence identity, to the sequence of one of the antibodies selected from the group consisting of pembrolizumab, nivolumab, avelumab, atezolizumab, durvalumab, spartalizumab, camrelizumab, sintilimab, tislelizumab, toripalimab, cemiplimab, and an antibody wherein the light chain sequences and the heavy chain sequences of the antibody correspond to SEQ ID NO: 7 and SEQ ID NO: 16, or to SEQ ID NO: 15 and SEQ ID NO: 14.

In some embodiments, the PD-1 inhibitor is an anti-PD-L1 antibody capable of inhibiting the interaction between PD-1 and PD-L1. In some embodiments, the anti-PD-L1 antibody comprises a heavy chain, which comprises three CDRs having amino acid sequences of SEQ ID NO: 19 (CDRH1), SEQ ID NO: 20 (CDRH2) and SEQ ID NO: 21 (CDRH3), and a light chain, which comprises three CDRs having amino acid sequences of SEQ ID NO: 22 (CDRL1), SEQ ID NO: 23 (CDRL2) and SEQ ID NO: 24 (CDRL3). In some embodiments, the anti-PD-L1 antibody comprises a heavy chain, which comprises three CDRs having amino acid sequences of SEQ ID NO: 1 (CDRH1), SEQ ID NO: 2 (CDRH2) and SEQ ID NO: 3 (CDRH3), and a light chain, which comprises three CDRs having amino acid sequences of SEQ ID NO: 4 (CDRL1), SEQ ID NO: 5 (CDRL2) and SEQ ID NO: 6 (CDRL3). In some embodiments, the light chain variable region and the heavy chain variable region of the anti-PD-L1 antibody comprise SEQ ID NO: 25 and SEQ ID NO: 26, respectively. In some embodiments, the light chain sequences and the heavy chain sequences of the anti-PD-L1 antibody correspond to SEQ ID NO: 7 and SEQ ID NO: 16, or to SEQ ID NO: 15 and SEQ ID NO: 14, respectively.

In some embodiments, the PD-1 inhibitor is an anti-PD-L1 antibody, wherein each of the light and heavy chain sequences have greater than or equal to 80% sequence identity, such as greater than or equal to 90% sequence identity, greater than or equal to 95% sequence identity, greater than or equal to 99% sequence identity, or 100% sequence identity with the amino acid sequence of the heavy and light chains of the antibody moiety of bintrafusp alfa and wherein the PD-1 inhibitor is still capable of binding to PD-L1. In some embodiments, the PD-1 inhibitor is an anti-PD-L1 antibody, wherein each of the light and heavy chain sequences have greater than or equal to 80% sequence identity, such as greater than or equal to 90% sequence identity, greater than or equal to 95% sequence identity, greater than or equal to 99% sequence identity, or 100% sequence identity with the amino acid sequence of the heavy and light chains of the antibody moiety of bintrafusp alfa and wherein the CDRs are fully identical with the CDRs of bintrafusp. In some embodiments, the PD-1 inhibitor is an anti-PD-L1 antibody with an amino acid sequence with not more than 50, not more than 40, or not more than 25 amino acid residues different from each of the heavy and light chain sequences of the antibody moiety of bintrafusp alfa and wherein the PD-1 inhibitor is still capable of binding to PD-L1. In some embodiments, the PD-1 inhibitor is an anti-PD-L1 antibody with an amino acid sequence with not more than 50, not more than 40, not more than 25, or not more than 10 amino acid residues different from each of the heavy and light chain sequences of the antibody moiety of bintrafusp alfa and wherein the CDRs are fully identical with the CDRs of bintrafusp alfa.

In some embodiments, the TGFβ inhibitor is capable of inhibiting the interaction between TGFβ and a TGFβ receptor; such as a TGFβ receptor, a TGFβ ligand- or receptor-blocking antibody, a small molecule inhibiting the interaction between TGFβ binding partners, and an inactive mutant TGFβ ligand that binds to the TGFβ receptor and competes for binding with endogenous TGFβ. In some embodiments, the TGFβ inhibitor is a soluble TGFβ receptor (e.g., a soluble TGFβ receptor II or Ill) or a fragment thereof capable of binding TGFβ. In some embodiments, the TGFβ inhibitor is an extracellular domain of human TGFβ receptor II (TGFβRII), or fragment thereof capable of binding TGFβ. In some embodiments, the TGFβRII corresponds to the wild-type human TGF-β Receptor Type 2 Isoform A sequence (e.g. the amino acid sequence of NCBI Reference Sequence (RefSeq) Accession No. NP_001020018 (SEQ ID NO: 9)), or the wild-type human TGF-β Receptor Type 2 Isoform B sequence (e.g., the amino acid sequence of NCBI RefSeq Accession No. NP_003233 (SEQ ID NO: 10)). In some embodiments, the TGFβ inhibitor comprises or consists of a sequence corresponding to SEQ ID NO: 11 or a fragment thereof capable of binding TGFβ. For instance, the TGFβ inhibitor may correspond to the full-length sequence of SEQ ID NO: 11. Alternatively, it may have an N-terminal deletion. For instance, the N-terminal 26 or less amino acids of SEQ ID NO: 11 may be deleted, such as 14-21 or 14-26 N-terminal amino acids. In some embodiments, the N-terminal 14, 19 or 21 amino acids of SEQ ID NO: 11 are deleted. In some embodiments, the TGFβ inhibitor comprises or consists of a sequence selected from the group consisting of SEQ ID NO: 11, SEQ ID NO: 12 and SEQ ID NO: 13. In some embodiments, the TGFβ inhibitor is a protein that is substantially identical, e.g., has at least 90% sequence identity, to the amino acid sequence of any one of SEQ ID NO: 11, SEQ ID NO: 12 and SEQ ID NO: 13 and is capable of binding TGFβ. In another embodiment, the TGFβ inhibitor is a protein that is substantially identical, e.g., has at least 90% sequence identity, to the amino acid sequence of SEQ ID NO: 11 and is capable of binding TGFβ. In one embodiment, the TGFβ inhibitor is a protein with an amino acid sequence that does not differ in more than 25 amino acids from SEQ ID NO: 11 and is capable of binding TGFβ.

In some embodiments, the TGFβ inhibitor is a protein that is substantially identical, e.g., has at least 90% sequence identity, to the amino acid sequence of the TGFβR of bintrafusp alfa and is still capable of binding TGFβ. In some embodiments, the TGFβ inhibitor is a protein with an amino acid sequence with not more than 50, not more than 40, or not more than 25 amino acid residues different from the TGFβR of bintrafusp alfa that is still capable of binding TGFβ. In some embodiments, the TGFβ inhibitor has 100-160 amino acid residues or 110-140 amino acid residues. In some embodiments, the amino acid sequence of the TGFβ inhibitor is selected from the group consisting of a sequence corresponding to positions 1-136 of the TGFβR of bintrafusp alfa, a sequence corresponding to positions 20-136 of the TGFβR of bintrafusp alfa and a sequence corresponding to positions 22-136 of the TGFβR of bintrafusp alfa.

In some embodiments, the TGFβ inhibitor is selected from the group consisting of lerdelimumab, XPA681, XPA089, LY2382770, LY3022859, 1D11, 2G7, AP11014, A-80-01, LY364947, LY550410, LY580276, LY566578, SB-505124, SD-093, SD-208, SB-431542, ISTH0036, ISTH0047, galunisertib (LY2157299 monohydrate, a small molecule kinase inhibitor of TGF-βRI), LY3200882 (a small molecule kinase inhibitor TGF-βRI disclosed by Pei et al. (2017) CANCER RES 77(13 Suppl):Abstract 955), metelimumab (an antibody targeting TGF-β1, see Colak et al. (2017) TRENDS CANCER 3(1):56-71), fresolimumab (GC-1008; an antibody targeting TGF-β1 and TGF-β2), XOMA 089 (an antibody targeting TGF-β1 and TGF-β2; see Mirza et al. (2014) INVESTIGATIVE OPHTHALMOLOGY & VISUAL SCIENCE 55:1121), AVID200 (a TGF-β1 and TGF-β3 trap, see Thwaites et al. (2017) BLOOD 130:2532), Trabedersen/AP12009 (a TGF-β2 antisense oligonucleotide, see Jaschinski et al. (2011) CURR PHARM BIOTECHNOL. 12(12):2203-13), Belagen-pumatucel-L (a tumor cell vaccine targeting TGF-β2, see, e.g., Giaccone et al. (2015) EUR J CANCER 51(16):2321-9), TGB-β pathway targeting agents described in Colak et al. (2017), supra, including Ki26894, SD208, SM16, IMC-TR1, PF-03446962, TEW-7197, and GW788388.

In some embodiments, the PD-1 inhibitor and the TGFβ inhibitor are fused, e.g., as an anti-PD(L)1:TGFβRII fusion protein. In some embodiments, the fusion molecule is an anti-PD-1:TGFβRII fusion protein or an anti-PD-L1:TGFβRII fusion protein. In some embodiments, the anti-PD(L)1:TGFβRII fusion protein is one of the anti-PD(L)1:TGFβRII fusion proteins disclosed in WO 2015/118175, WO 2018/205985, WO 2020/014285 or WO 2020/006509. In some embodiments, the N-terminal end of the sequence of the TGFβRII or the fragment thereof is fused to the C-terminal end of each heavy chain sequence of the antibody or fragment thereof. In some embodiments, the antibody or the fragment thereof and the extracellular domain of TGFβRII or the fragment thereof are genetically fused via a linker sequence. In some embodiments, the linker sequence is a short, flexible peptide. In one embodiment, the linker sequence is (G₄S)ₓG, wherein x is 3-6, such as 4-5 or 4.

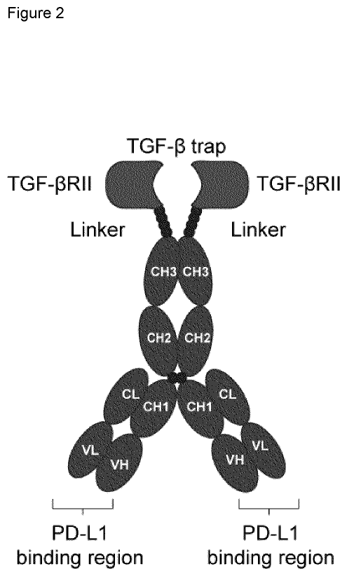

An exemplary anti-PD-L1:TGFβRII fusion protein is shown in FIG. 2. The depicted heterotetramer consists of the two light chain sequences of the anti-PD-L1 antibody, and two sequences each comprising a heavy chain sequence of the anti-PD-L1 antibody which C-terminus is genetically fused via a linker sequence to the N-terminus of the extracellular domain of the TGFβRII or the fragment thereof.

In one embodiment, the extracellular domain of TGFβRII or the fragment thereof of the anti-PD(L)1:TGFβRII fusion protein has an amino acid sequence that does not differ in more than 25 amino acids from SEQ ID NO: 11 and is capable of binding TGFβ. In some embodiments, the anti-PD-L1:TGFβRII fusion protein is one of the anti-PD-L1:TGFβRII fusion proteins disclosed in WO 2015/118175, WO 2018/205985 or WO 2020/006509. For instance, the anti-PD-L1:TGFβRII fusion protein may comprise the light chain sequences and heavy chain sequences of SEQ ID NO: 1 and SEQ ID NO: 3 of WO 2015/118175, respectively. In another embodiment, the anti-PD-L1:TGFβRII fusion protein is one of the constructs listed in Table 2 of WO 2018/205985, such as construct 9 or 15 thereof. In other embodiments, the antibody having the heavy chain sequences of SEQ ID NO: 11 and the light chain sequences of SEQ ID NO: 12 of WO 2018/205985 is fused via a linking sequence (G4S)ₓG, wherein x is 4-5, to the TGFβRII extracellular domain sequence of SEQ ID NO: 14 (wherein “x” of the linker sequence is 4) or SEQ ID NO: 15 (wherein “x” of the linker sequence is 5) of WO 2018/205985. In another embodiment, the anti-PD-L1:TGFβRII fusion protein is SHR1701. In a further embodiment, the anti-PD-L1:TGFβRII fusion protein is one of the fusion molecules disclosed in WO 2020/006509. In one embodiment, the anti-PD-L1:TGFβRII fusion protein is Bi-PLB-1, Bi-PLB-2 or Bi-PLB-1.2 disclosed in WO 2020/006509. In one embodiment, the anti-PD-L1:TGFβRII fusion protein is Bi-PLB-1.2 disclosed in WO 2020/006509. In one embodiment, the anti-PD-L1:TGFβRII fusion protein comprises SEQ ID NO:128 and SEQ ID NO:95 disclosed in WO 2020/006509. In some embodiments, the amino acid sequence of the light chain sequences and the heavy chain sequences of the anti-PD-L1:TGFβRII fusion protein respectively correspond to the light chain sequences and the heavy chain sequences selected from the group consisting of: (1) SEQ ID NO: 7 and SEQ ID NO: 8, (2) SEQ ID NO: 15 and SEQ ID NO: 17, (3) SEQ ID NO: 15 and SEQ ID NO: 18 of the present disclosure and (4) SEQ ID NO:128 and SEQ ID NO:95 disclosed in WO 2020/006509. In some embodiments, the anti-PD-L1:TGFβRII fusion protein is still capable of binding PD-L1 and TGFβ and the amino acid sequence of its light chain sequences and heavy chain sequences are respectively substantially identical, e.g., have at least 90% sequence identity, to the light chain sequences and the heavy chain sequences selected from the group consisting of: (1) SEQ ID NO: 7 and SEQ ID NO: 8, (2) SEQ ID NO: 15 and SEQ ID NO: 17, (3) SEQ ID NO: 15 and SEQ ID NO: 18 of the present disclosure and (4) SEQ ID NO:128 and SEQ ID NO:95 disclosed in WO 2020/006509. In some embodiments, the amino acid sequence of the light chain sequences and the heavy chain sequences of the PD-1 inhibitor of the anti-PD-L1:TGFβRII fusion protein are respectively not more than 50, not more than 40, not more than 25, or not more than 10 amino acid residues different from the light chain sequences and the heavy chain sequences of the antibody moiety of bintrafusp alfa and the CDRs are fully identical with the CDRs of bintrafusp alfa and/or the PD-1 inhibitor is still capable of binding to PD-L1. In some embodiments, the amino acid sequence of the anti-PD-L1:TGFβRII fusion protein is substantially identical, e.g., has at least 90% sequence identity, to the amino acid sequence of bintrafusp alfa and is capable of binding to PD-L1 and TGF-β. In some embodiments, the amino acid sequence of the anti-PD-L1:TGFβRII fusion protein corresponds to the amino acid sequence of bintrafusp alfa. In some embodiments, the anti-PD-L1:TGFβRII fusion protein is bintrafusp alfa.

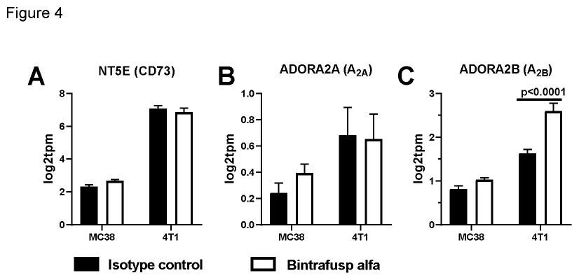

In a particular embodiment, the anti-PD-1:TGFβRII fusion protein is one of the fusion molecules disclosed in WO 2020/014285 that binds both PD-1 and TGF-β, e.g. as depicted in FIG. 4 therein or as described in Example 1, including those identified in Tables 2-9, as specified in table 16, therein, and in particular a fusion protein that binds both PD-1 and TGF-β and comprising a sequence that is substantially identical, e.g., has at least 90% sequence identity, to SEQ ID NO:15 or SEQ ID NO:296 and a sequence that is substantially identical, e.g., has at least 90% sequence identity, to SEQ ID NO:16, SEQ ID NO:143, SEQ ID NO:144, SEQ ID NO:145, SEQ ID NO:294 or SEQ ID NO:295 therein. In an embodiment, the anti-PD-1:TGFβIIR fusion protein comprises SEQ ID NO:15 and SEQ ID NO:16 of WO 2020/014285. In an embodiment, the anti-PD-1:TGFβIIR fusion protein comprises SEQ ID NO:15 and SEQ ID NO:143 of WO 2020/014285. In an embodiment, the anti-PD-1:TGFβIIR fusion protein comprises SEQ ID NO:15 and SEQ ID NO:144 of WO 2020/014285. In an embodiment, the anti-PD-1:TGFβIIR fusion protein comprises SEQ ID NO:15 and SEQ ID NO:145 of WO 2020/014285. In an embodiment, the anti-PD-1:TGFβIIR fusion protein comprises SEQ ID NO:15 and SEQ ID NO:294 of WO 2020/014285. In an embodiment, the anti-PD-1:TGFβIIR fusion protein comprises SEQ ID NO:15 and SEQ ID NO:295 of WO 2020/014285. In an embodiment, the anti-PD-1:TGFβIIR fusion protein comprises SEQ ID NO:296 and SEQ ID NO:16 of WO 2020/014285. In an embodiment, the anti-PD-1:TGFβIIR fusion protein comprises SEQ ID NO:296 and SEQ ID NO:143 of WO 2020/014285. In an embodiment, the anti-PD-1:TGFβIIR fusion protein comprises SEQ ID NO:296 and SEQ ID NO:144 of WO 2020/014285. In an embodiment, the anti-PD-1:TGFβIIR fusion protein comprises SEQ ID NO:296 and SEQ ID NO:145 of WO 2020/014285. In an embodiment, the anti-PD-1:TGFβIIR fusion protein comprises SEQ ID NO:296 and SEQ ID NO:294 of WO 2020/014285. In an embodiment, the anti-PD-1:TGFβIIR fusion protein comprises SEQ ID NO:296 and SEQ ID NO:295 of WO 2020/014285. In a further embodiment, the anti-PD-1:TGFβIIR fusion protein is one of the fusion molecules disclosed in WO 2020/006509. In one embodiment, the anti-PD-1:TGFβIIR fusion protein is Bi-PB-1, Bi-PB-2 or Bi-PB-1.2 disclosed in WO 2020/006509. In one embodiment, the anti-PD-1:TGFβIIR fusion protein is Bi-PB-1.2 disclosed in WO 2020/006509. In one embodiment, the anti-PD-1:TGFβIIR fusion protein comprises SEQ ID NO:108 and SEQ ID NO:93 disclosed in WO 2020/006509.

In some embodiments, the adenosine inhibitor is an adenosine receptor inhibitor. In some embodiments, the adenosine inhibitor is an adenosine A_{2A} and/or A_{2B} receptor inhibitor. In some embodiments, the adenosine A_{2A} and/or A_{2B} receptor inhibitor is a small molecule that competitively inhibits the binding of adenosine to the adenosine A_{2A} and/or A_{2B} receptor.

In some embodiments, the adenosine inhibitor is an adenosine A_{2A} receptor inhibitor. In some embodiments, the adenosine A_{2A} receptor inhibitor is selected from the group consisting of Istradefylline (KW-6002), Preladenant (SCH420814), Ciforadenant, SCH58261, SCH-442,416, ZM-241,385, CGS-15943, Tozadenant, Vipadenant (V-2006), V-81444 (CPI-444), AZD-4635 (HTL-1071), NIR-178 (PBF-509), Medi-9447, PNQ-370, ZM-241385, ASO-5854, ST-1535, ST-4206, DT1133, DT-0926, MK-3814, CGS-2168, CGS-21680, ZM241385 and NECA.

In some embodiments, the adenosine inhibitor is an adenosine A_{2B} receptor inhibitor. In some embodiments, the adenosine A_{2B} receptor inhibitor is selected from the group consisting of xanthines (DPSPX (1,3-dipropyl-8-sulphophenylxanthine), DPCPX (1,3-diproyl-8c-yclopentylxanthine), DPX (1,3 diethylphenylxanthine), the antiasthmatic drug enprofylline (3-n-propylxanthine)), the non-xanthine compound 2,4-dioxobenzopteridine (alloxazine), ATL801, CVT-6833, PSB-603, PSB-605, PSB-0788, PSB-1115, ISAM-140, GS6201, MRS1706 and MRS1754.

In some embodiments, the adenosine inhibitor is an adenosine A_{2A} and A_{2B} receptor inhibitor. In some embodiments, the adenosine A_{2A} and A_{2B} receptor inhibitor is a thiazolopyridine derivative. In some embodiments, the adenosine A_{2A} and A_{2B} receptor inhibitor is selected from the group consisting of AB928 and one of the adenosine A_{2A} and A_{2B} receptor inhibitors disclosed in WO 2019/038214, WO 2019/038215, WO 2019/025099, WO 2020/083878, WO 2020/083856 and WO 2020/152132, in particular, selected from the compounds as referred to in the claims of these publications.

In some embodiments, the adenosine receptor inhibitor is selected from the following embodiments E1-E13:
E1. Compound of the formula I,

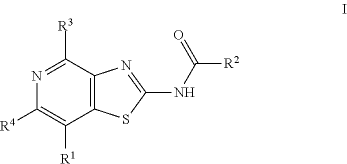

wherein
R¹ is linear or branched alkyl having 1-10 C atoms which is unsubstituted or mono-, di- or trisubstituted by R⁵ and in which 1-4 C atoms may be replaced, independently of one another, by O, S, SO, SO₂, NH, NCH₃, —OCO—, —NHCONH—, —NHCO—, —NR⁶SO₂R⁷—, —COO—, —CONH—, —NCH₃CO—, —CONCH₃—, —C≡C— groups and/or —CH═CH— groups, and/or, in addition, 1-10 H atoms may be replaced by F and/or Cl, or mono- or bicyclic cyclic alkyl having 3-7 C atoms which is unsubstituted or mono-, di- or trisubstituted by R⁵ and in which 1-4 C atoms may be replaced, independently of one another, by O, S, SO, SO₂, NH, NCH₃, —OCO—, —NHCONH—, —NHCO—, —NR⁶SO₂R⁷—, —COO—, —CONH—, —NCH₃CO—, —CONCH₃—, —C≡C— groups and/or by —CH═CH— groups and/or, in addition, 1-10 H atoms may be replaced by F and/or Cl, or mono- or bicyclic heteroaryl, heterocyclyl, aryl or cyclic alkylaryl, containing 3 to 14 carbon atoms and 0-4 heteroatoms, independently selected from N, O and S, which is unsubstituted or mono-, di- or trisubstituted by R⁵,
R² is linear or branched alkyl having 1-10 C atoms which is unsubstituted or mono-, di- or trisubstituted by R⁵ and in which 1-4 C atoms may be replaced, independently of one another, by O, S, SO, SO₂, NH, NCH₃, —OCO—, —NHCONH—, —NHCO—, —NR⁶SO₂R⁷—, —COO—, —CONH—, —NCH₃CO—, —CONCH₃—, —C≡C— groups and/or —CH═CH— groups, and/or, in addition, 1-10 H atoms may be replaced by F and/or Cl, or cyclic alkyl having 3-7 C atoms which is unsubstituted or mono-, di- or trisubstituted by R⁵ and in which 1-4 C atoms may be replaced, independently of one another, by O, S, SO, SO₂, NH, NCH₃, —OCO—, —NHCONH—, —NHCO—, —NR⁶SO₂R⁷—, —COO—, —CONH—, —NCH₃CO—, —CONCH₃—, —C≡C— groups and/or by —CH═CH— groups and/or, in addition, 1-11 H atoms may be replaced by F and/or Cl, or mono- or bicyclic heteroaryl, heterocyclyl, aryl or cyclic alkylaryl, containing 3 to 14 carbon atoms and 0-4 heteroatoms, independently selected from N, O and S, which is unsubstituted or mono-, di- or trisubstituted by R⁵,
R³ is linear or branched alkyl or O-alkyl having 1-6 C atoms or cyclic alkyl having 3-6 C atoms, which is unsubstituted or mono-, di- or trisubstituted by H, ═S, ═NH, ═O, OH, cyclic alkyl having 3-6 C atoms, COOH, Hal, NH₂, SO₂CH₃, SO₂NH₂, CN, CONH₂, NHCOCH₃, NHCONH₂ or NO₂,
R⁴ is H, D, linear or branched alkyl having 1-6 C atoms or Hal,
R⁵ is H, R⁶, ═S, ═NR⁶, ═O, OH, COOH, Hal, NH₂, SO₂CH₃, SO₂NH₂, CN, CONH₂, NHCOCH₃, NHCONH₂, NO₂, or linear or branched alkyl having 1-10 C atoms which is unsubstituted or mono-, di- or trisubstituted by R⁶ and in which 1-4 C atoms may be replaced, independently of one another, by O, S, SO, SO₂, NH, NCH₃, —OCO—, —NHCONH—, —NHCO—, —NR⁶SO₂R⁷—, —COO—, —CONH—, —NCH₃CO—, —CONCH₃—, —C≡C— groups and/or —CH═CH— groups, and/or, in addition, 1-10 H atoms may be replaced by F and/or Cl, or mono- or bicyclic cyclic alkyl having 3-7 C atoms which is unsubstituted or mono-, di- or trisubstituted by R⁶ and in which 1-4 C atoms may be replaced, independently of one another, by O, S, SO, SO₂, NH, NCH₃, —OCO—, —NHCONH—, —NHCO—, —NR⁶SO₂R⁷—, —COO—, —CONH—, —NCH₃CO—, —CONCH₃—, —C≡C— groups and/or by —CH═CH— groups and/or, in addition, 1-10 H atoms may be replaced by F and/or Cl, or mono- or bicyclic heteroaryl, heterocyclyl, aryl or cyclic alkylaryl, containing 3 to 14 carbon atoms and 0-4 heteroatoms, independently selected from N, O and S, which is unsubstituted or mono-, di- or trisubstituted by R⁶,
R⁶, R⁷ are independently of one another selected from the group consisting of H, ═S, ═NH, ═O, OH, COOH, Hal, NH₂, SO₂CH₃, SO₂NH₂, CN, CONH₂, NHCOCH₃, NHCONH₂, NO₂ and linear or branched alkyl having 1-10 C atoms in which 1-4 C atoms may be replaced, independently of one another, by O, S, SO, SO₂, NH, NCH₃, —OCO—, —NHCONH—, —NHCO—, —COO—, —CONH—, —NCH₃CO—, —CONCH₃—, —C≡C— groups and/or —CH═CH— groups, and/or, in addition, 1-10 H atoms may be replaced by F and/or Cl,
Hal is F, Cl, Br, or I,
D is deuterium
and pharmaceutically acceptable salts, derivatives, solvates, prodrugs and stereoisomers thereof, including mixtures thereof in all ratios.
E2. Compound according to embodiment E1, wherein
R¹ is linear or branched alkyl having 1-10 C atoms which is unsubstituted or mono-, di- or trisubstituted by R⁴ and in which 1-4 C atoms may be replaced, independently of one another, by O, S, SO, SO₂, NH, NCH₃, —OCO—, —NHCONH—, —NHCO—, —NR⁵SO₂R⁶—, —COO—, —CONH—, —NCH₃CO—, —CONCH₃—, —C≡C— groups and/or —CH═CH— groups, and/or, in addition, 1-10 H atoms may be replaced by F and/or Cl, or one of the following structures:

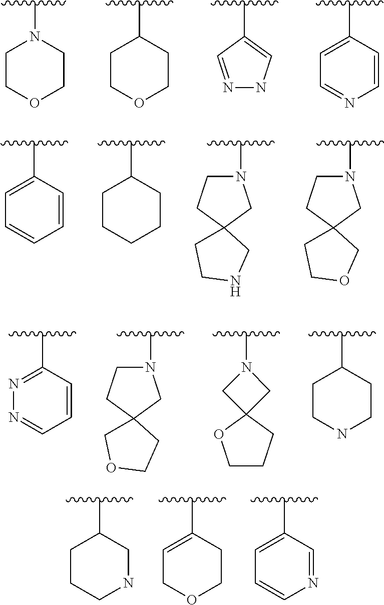

which is unsubstituted or mono-, di- or trisubstituted with R⁵
and wherein R², R³, R⁴, R⁵, R⁶ and R⁷ have the meanings as disclosed in embodiment E1, and pharmaceutically acceptable salts, derivatives, solvates, prodrugs and stereoisomers thereof, including mixtures thereof in all ratios.
E3. Compound according to embodiment E1 or embodiment E2, wherein
R¹ is one of the following structures:

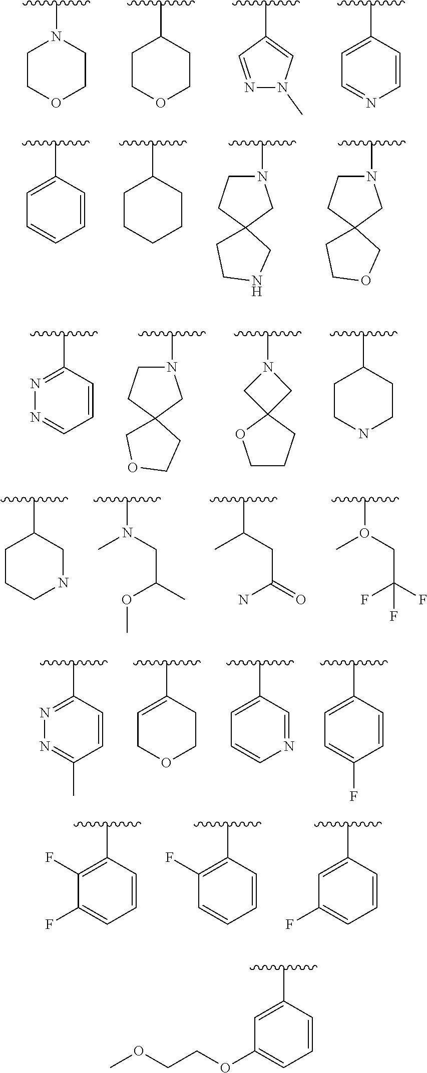

and wherein R², R³, R⁴, R⁵, R⁶ and R⁷ have the meanings as disclosed in embodiment E1, and pharmaceutically acceptable salts, derivatives, solvates, prodrugs and stereoisomers thereof, including mixtures thereof in all ratios.
E4. Compound according to any one of embodiments E1 to E3, wherein
R¹ is phenyl, methylpyrazole or dihydropyran
and R², R³, R⁴, R⁵, R⁶ and R⁷ have the meanings as in embodiment E1, and pharmaceutically acceptable salts, derivatives, solvates, prodrugs and stereoisomers thereof, including mixtures thereof in all ratios.
E5. Compound according to any one of embodiments E1 to E4, wherein
R² is one of the following structures:

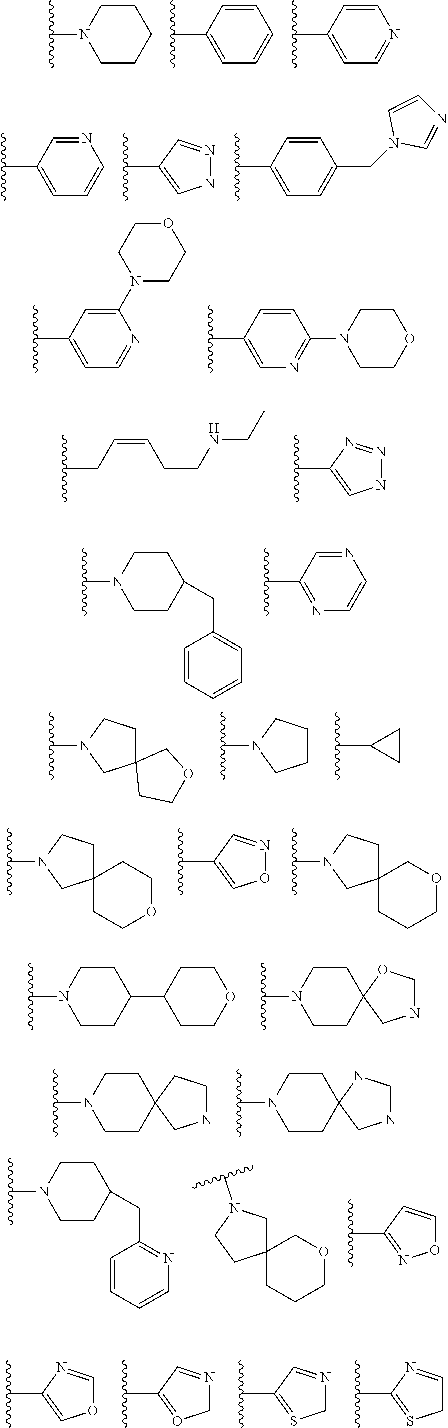

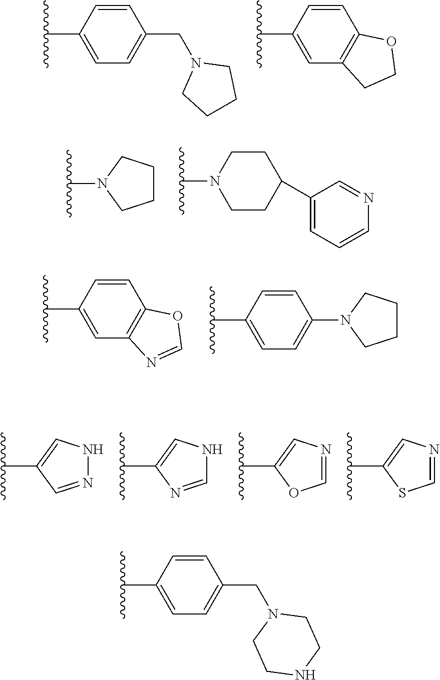

which is unsubstituted or mono-, di- or trisubstituted with R⁵
and wherein R¹, R³, R⁴, R⁵, R⁶ and R⁷ have the meanings as disclosed in embodiment E1, and pharmaceutically acceptable salts, derivatives, solvates, prodrugs and stereoisomers thereof, including mixtures thereof in all ratios.
E6. Compound according to any one of embodiments E1 to E5, wherein
R² is one of the following structures:

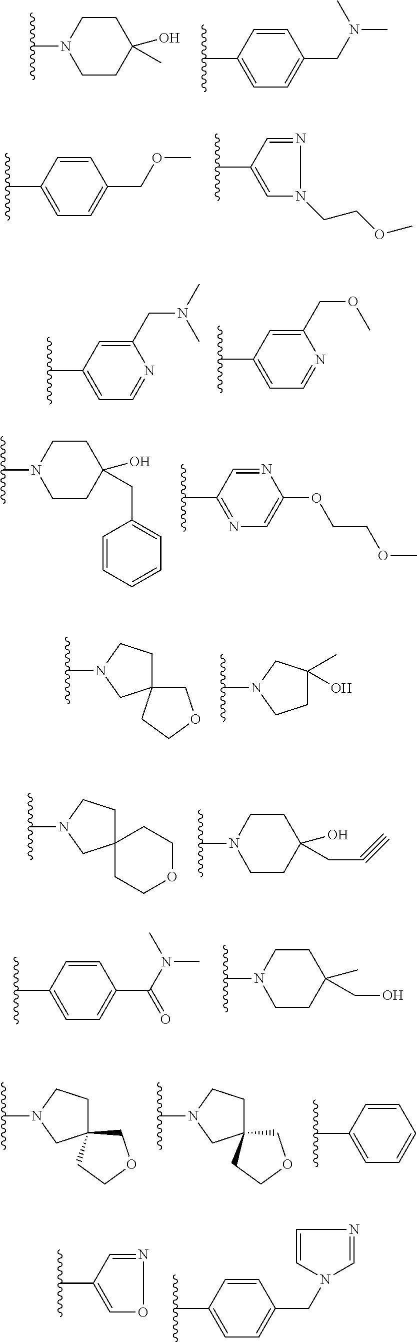

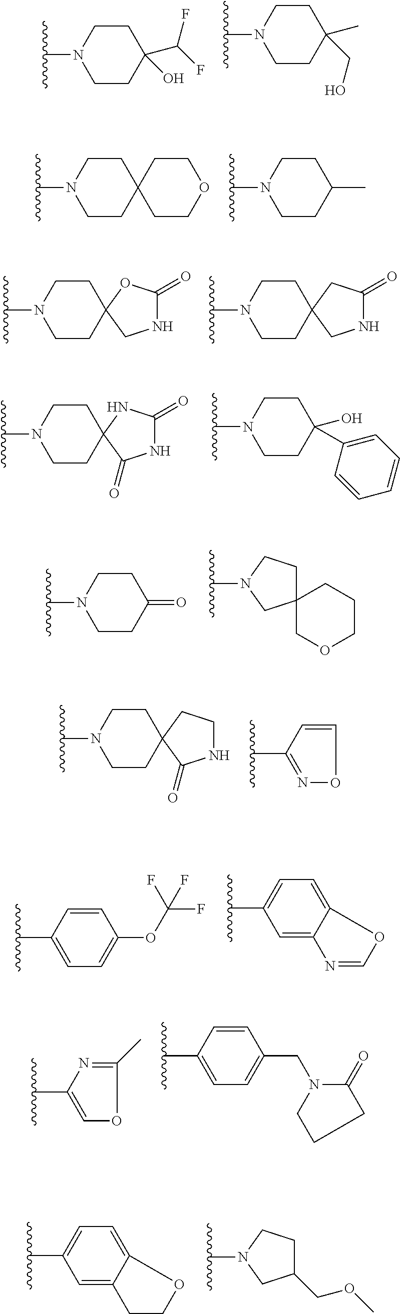

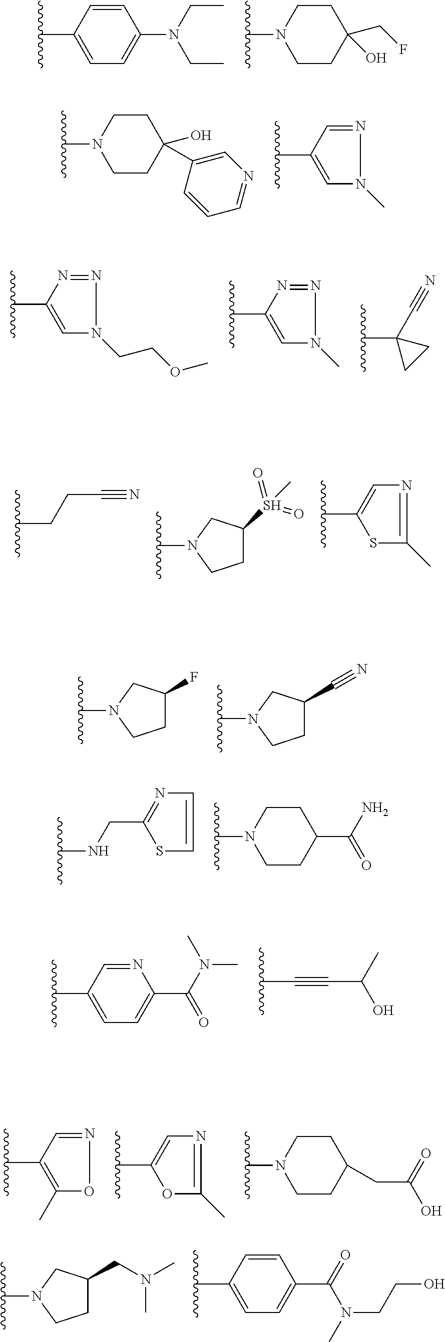

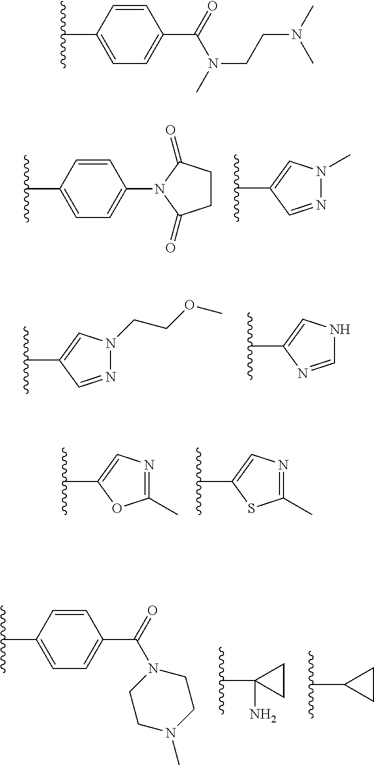

and wherein R¹, R³, R⁴, R⁵, R⁶ and R⁷ have the meanings as disclosed in embodiment E1, and pharmaceutically acceptable salts, derivatives, solvates, prodrugs and stereoisomers thereof, including mixtures thereof in all ratios.
E7. Compound according to any one of embodiments E1 to E6, wherein
R³ one of the following structures

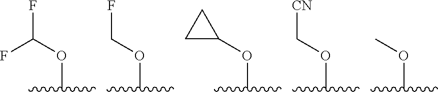

and R¹, R², R⁴, R⁵, R⁶ and R⁷ have the meanings as disclosed in embodiment E1, and pharmaceutically acceptable salts, derivatives, solvates, prodrugs and stereoisomers thereof, including mixtures thereof in all ratios.
E8. Compound according to any one of embodiments E1 to E7, wherein
R³ is OMe
and R¹, R², R⁴, R⁵, R⁶ and R⁷ have the meanings as disclosed in embodiment E1, and pharmaceutically acceptable salts, derivatives, solvates, prodrugs and stereoisomers thereof, including mixtures thereof in all ratios.
E9. Compound according to any one of embodiments E1 to E8, wherein
R¹ is phenyl, methylpyrazole or dihydropyran,
R³ is OMe
and R², R⁴, R⁵, R⁶ and R⁷ have the meanings as disclosed in embodiment E1, and pharmaceutically acceptable salts, derivatives, solvates, prodrugs and stereoisomers thereof, including mixtures thereof in all ratios.
E10. Compound according to embodiment E1, wherein
R⁴ is H, D, methyl, ethyl, F, Br or Cl
and wherein R¹, R², R³, R⁵, R⁶ and R⁷ have the meanings as disclosed in embodiment E1, and pharmaceutically acceptable salts, derivatives, solvates, prodrugs and stereoisomers thereof, including mixtures thereof in all ratios.

E11. Compound according to embodiment E1 or E2, wherein
R⁴ is H,
and wherein R¹, R², R³, R⁵, R⁶ and R⁷ have the meanings as disclosed in embodiment E1, and pharmaceutically acceptable salts, derivatives, solvates, prodrugs and stereoisomers thereof, including mixtures thereof in all ratios.
E12. Compound selected from the group of compounds of Table 1:

TABLE 1

| | |
|---|---|
| 1 | 4-Hydroxy-4-methyl-piperidine-1-carboxylic acid (4- |
| | methoxy-7-morpholin-4-yl-thiazolo[4,5-c]pyridin-2-yl)-amide |
| 2 | 4-Dimethylaminomethyl-N-(4-methoxy-7-morpholin-4- |
| | yl-thiazolo[4,5-c]pyridin-2-yl)-benzamide |
| 3 | 4-Methoxymethyl-N-(4-methoxy-7-morpholin-4-yl- |
| | thiazolo[4,5-c]pyridin-2-yl)-benzamide |
| 4 | 1-(2-Methoxy-ethyl)-1H-pyrazole-4-carboxylic acid (4- |
| | methoxy-7-morpholin-4-yl-thiazolo[4,5-c]pyridin-2-yl)-amide |
| 5 | 2-Dimethylaminomethyl-N-(4-methoxy-7-morpholin-4- |
| | yl-thiazolo[4,5-c]pyridin-2-yl)-isonicotinamide |
| 6 | 2-Methoxymethyl-N-(4-methoxy-7-morpholin-4-yl- |
| | thiazolo[4,5-c]pyridin-2-yl)-isonicotinamide |
| 7 | 4-benzyl-4-hydroxy-N-[4-methoxy-7-(morpholin-4-yl)- |
| | [1,3]thiazolo[4,5-c]pyridin-2-yl]piperidine-1-carboxamide |
| 8 | N-[4-methoxy-7-(morpholin-4-yl)-[1,3]thiazolo[4,5- |
| | c]pyridin-2-yl]-5-(2-methoxyethoxy)pyrazine-2-carboxamide |
| 9 | 2-Oxa-7-aza-spiro[4.4]nonane-7-carboxylic acid (4- |
| | methoxy-7-morpholin-4-yl-thiazolo[4,5-c]pyridin-2-yl)-amide |
| 10 | 3-Hydroxy-3-methyl-pyrrolidine-1-carboxylic acid (4- |
| | methoxy-7-morpholin-4-yl-thiazolo[4,5-c]pyridin-2-yl)-amide |
| 11 | 8-Oxa-2-aza-spiro[4.5]decane-2-carboxylic acid (4- |
| | methoxy-7-morpholin-4-yl-thiazolo[4,5-c]pyridin-2-yl)-amide |
| 12 | 4-Hydroxy-4-prop-2-ynyl-piperidine-1-carboxylic acid |
| | (4-methoxy-7-morpholin-4-yl-thiazolo[4,5-c]pyridin-2-yl)-amide |
| 13 | 1-(2-Methoxy-ethyl)-1H-pyrazole-4-carboxylic acid [4- |
| | methoxy-7-(1-methyl-1H-pyrazol-4-yl)-thiazolo[4,5-c]pyridin-2- |
| | yl]-amide |
| 14 | 4-Methoxymethyl-N-[4-methoxy-7-(1-methyl-1H- |
| | pyrazol-4-yl)-thiazolo[4,5-c]pyridin-2-yl]-benzamide |
| 15 | N-(4-Methoxy-7-morpholin-4-yl-thiazolo[4,5-c]pyridin- |
| | 2-yl)-N′,N′-dimethyl-terephthalamide |
| 16 | 4-Hydroxymethyl-4-methyl-piperidine-1-carboxylic acid |
| | (4-methoxy-7-morpholin-4-yl-thiazolo[4,5-c]pyridin-2-yl)-amide |
| 17 | (5S)-N-[4-methoxy-7-(morpholin-4-yl)-[1,3]thiazolo[4,5- |
| | c]pyridin-2-yl]-2-oxa-7-azaspiro[4.4]nonane-7-carboxamide |
| 18 | (5R)-N-[4-methoxy-7-(morpholin-4-yl)-[1,3]thiazolo[4,5- |
| | c]pyridin-2-yl]-2-oxa-7-azaspiro[4.4]nonane-7-carboxamide |
| 19 | N-[4-Methoxy-7-(1-methyl-1H-pyrazol-4-yl)- |
| | thiazolo[4,5-c]pyridin-2-yl]-benzamide |
| 20 | 4-Methoxy-7-(1-methyl-1H-pyrazol-4-yl)-thiazolo[4,5- |
| | c]pyridin-2-ylamine |
| 21 | 4-Hydroxy-4-methyl-piperidine-1-carboxylic acid [4- |
| | methoxy-7-(1-methyl-1H-pyrazol-4-yl)-thiazolo[4,5-c]pyridin-2- |
| | yl]-amide |
| 22 | 4-Dimethylaminomethyl-N-[4-methoxy-7-(1-methyl-1H- |
| | pyrazol-4-yl)-thiazolo[4,5-c]pyridin-2-yl]-benzamide |
| 23 | 4-Methoxymethyl-N-[4-methoxy-7-(tetrahydro-pyran-4- |
| | yl)-thiazolo[4,5-c]pyridin-2-yl]-benzamide |
| 24 | 2-Dimethylaminomethyl-N-[4-methoxy-7-(1-methyl-1H- |
| | pyrazol-4-yl)-thiazolo[4,5-c]pyridin-2-yl]-isonicotinamide |
| 25 | 2-Dimethylaminomethyl-N-[4-methoxy-7-(tetrahydro- |
| | pyran-4-yl)-thiazolo[4,5-c]pyridin-2-yl]-isonicotinamide |
| 26 | 2-Methoxymethyl-N-[4-methoxy-7-(1-methyl-1H- |
| | pyrazol-4-yl)-thiazolo[4,5-c]pyridin-2-yl]-isonicotinamide |
| 27 | 4-Dimethylaminomethyl-N-[4-methoxy-7-(tetrahydro- |
| | pyran-4-yl)-thiazolo[4,5-c]pyridin-2-yl]-benzamide |
| 28 | 2-Methoxymethyl-N-[4-methoxy-7-(tetrahydro-pyran-4- |
| | yl)-thiazolo[4,5-c]pyridin-2-yl]-isonicotinamide |
| 29 | 1-(2-Methoxy-ethyl)-1H-pyrazole-4-carboxylic acid [4- |
| | methoxy-7-(tetrahydro-pyran-4-yl)-thiazolo[4,5-c]pyridin-2-yl]- |
| | amide |
| 30 | 4-Hydroxy-4-methyl-piperidine-1-carboxylic acid [4- |
| | methoxy-7-(tetrahydro-pyran-4-yl)-thiazolo[4,5-c]pyridin-2-yl]- |
| | amide |
| 31 | Isoxazole-4-carboxylic acid (4-methoxy-7-morpholin-4- |
| | yl-thiazolo[4,5-c]pyridin-2-yl)-amide |
| 32 | 4-Hydroxy-4-methyl-piperidine-1-carboxylic acid (4- |
| | fluoromethoxy-7-morpholin-4-yl-thiazolo[4,5-c]pyridin-2-yl)- |
| | amide |
| 33 | 4-Imidazol-1-ylmethyl-N-(4-methoxy-7-morpholin-4-yl- |
| | thiazolo[4,5-c]pyridin-2-yl)-benzamide |
| 34 | 7-Oxa-2-aza-spiro[4.5]decane-2-carboxylic acid (4- |
| | methoxy-7-morpholin-4-yl-thiazolo[4,5-c]pyridin-2-yl)-amide |
| 35 | 1-(2-Methoxy-ethyl)-1H-pyrazole-4-carboxylic acid (4- |
| | methoxy-7-pyridin-4-yl-thiazolo[4,5-c]pyridin-2-yl)-amide |
| 36 | 1-(2-Methoxy-ethyl)-1H-pyrazole-4-carboxylic acid [4- |
| | methoxy-7-(6-methyl-pyridazin-3-yl)-thiazolo[4,5-c]pyridin-2- |
| | yl]-amide |
| 37 | 4-Hydroxy-4-methyl-piperidine-1-carboxylic acid (4- |
| | methoxy-7-phenyl-thiazolo[4,5-c]pyridin-2-yl)-amide |
| 38 | 4-Difluoromethyl-4-hydroxy-piperidine-1-carboxylic |
| | acid (4-methoxy-7-phenyl-thiazolo[4,5-c]pyridin-2-yl)-amide |
| 39 | 4-Hydroxymethyl-4-methyl-piperidine-1-carboxylic acid |
| | (4-methoxy-7-phenyl-thiazolo[4,5-c]pyridin-2-yl)-amide |
| 40 | 4-Fluoromethyl-4-hydroxy-piperidine-1-carboxylic acid |
| | (4-methoxy-7-phenyl-thiazolo[4,5-c]pyridin-2-yl)-amide |
| 41 | 3-Oxa-9-aza-spiro[5.5]undecane-9-carboxylic acid (4- |
| | methoxy-7-phenyl-thiazolo[4,5-c]pyridin-2-yl)-amide |
| 42 | 4-Methyl-piperidine-1-carboxylic acid (4-methoxy-7- |
| | phenyl-thiazolo[4,5-c]pyridin-2-yl)-amide |
| 43 | 4-Benzyl-4-hydroxy-piperidine-1-carboxylic acid (4- |
| | methoxy-7-phenyl-thiazolo[4,5-c]pyridin-2-yl)-amide |
| 44 | 2-Oxo-1-oxa-3,8-diaza-spiro[4.5]decane-8-carboxylic |
| | acid (4-methoxy-7-phenyl-thiazolo[4,5-c]pyridin-2-yl)-amide |
| 45 | 3-Oxo-2,8-diaza-spiro[4.5]decane-8-carboxylic acid (4- |
| | methoxy-7-phenyl-thiazolo[4,5-c]pyridin-2-yl)-amide |
| 46 | 4-Dimethylaminomethyl-N-(4-methoxy-7-phenyl- |
| | thiazolo[4,5-c]pyridin-2-yl)-benzamide |
| 47 | 4-Methoxymethyl-N-(4-methoxy-7-phenyl-thiazolo[4,5- |
| | c]pyridin-2-yl)-benzamide |
| 48 | 2,4-Dioxo-1,3,8-triaza-spiro[4.5]decane-8-carboxylic |
| | acid (4-methoxy-7-phenyl-thiazolo[4,5-c]pyridin-2-yl)-amide |
| 49 | 4′-Hydroxy-3′,4′,5′,6′-tetrahydro-2′H-[3,4′]bipyridinyl-1′- |
| | carboxylic acid (4-methoxy-7-phenyl-thiazolo[4,5-c]pyridin-2- |
| | yl)-amide |
| 50 | 4-Oxo-piperidine-1-carboxylic acid (4-methoxy-7- |
| | phenyl-thiazolo[4,5-c]pyridin-2-yl)-amide |
| 51 | 1-Oxo-2,8-diaza-spiro[4.5]decane-8-carboxylic acid (4- |
| | methoxy-7-phenyl-thiazolo[4,5-c]pyridin-2-yl)-amide |
| 52 | 7-Oxa-2-aza-spiro[4.5]decane-2-carboxylic acid (4- |
| | methoxy-7-phenyl-thiazolo[4,5-c]pyridin-2-yl)-amide |
| 53 | 8-Oxa-2-aza-spiro[4.5]decane-2-carboxylic acid (4- |
| | methoxy-7-phenyl-thiazolo[4,5-c]pyridin-2-yl)-amide |
| 54 | 2-Oxa-7-aza-spiro[4.4]nonane-7-carboxylic acid (4- |
| | methoxy-7-phenyl-thiazolo[4,5-c]pyridin-2-yl)-amide |
| 55 | 4-Imidazol-1-ylmethyl-N-(4-methoxy-7-phenyl- |
| | thiazolo[4,5-c]pyridin-2-yl)-benzamide |
| 56 | Isoxazole-3-carboxylic acid (4-methoxy-7-phenyl- |
| | thiazolo[4,5-c]pyridin-2-yl)-amide |
| 57 | 4-Hydroxy-4-prop-2-ynyl-piperidine-1-carboxylic acid |
| | (4-methoxy-7-phenyl-thiazolo[4,5-c]pyridin-2-yl)-amide |
| 58 | N-(4-Methoxy-7-phenyl-thiazolo[4,5-c]pyridin-2-yl)- |
| | N′,N′-dimethyl-terephthalamide |
| 59 | N-(4-Methoxy-7-phenyl-thiazolo[4,5-c]pyridin-2-yl)-4- |
| | trifluoromethoxy-benzamide |
| 60 | 2-Methyl-oxazole-4-carboxylic acid (4-methoxy-7- |
| | phenyl-thiazolo[4,5-c]pyridin-2-yl)-amide |
| 61 | Benzooxazole-5-carboxylic acid (4-methoxy-7-phenyl- |
| | thiazolo[4,5-c]pyridin-2-yl)-amide |
| 62 | N-(4-Methoxy-7-phenyl-thiazolo[4,5-c]pyridin-2-yl)-4- |
| | (2-oxo-pyrrolidin-1-ylmethyl)-benzamide |
| 63 | 2,3-Dihydro-benzofuran-5-carboxylic acid (4-methoxy- |
| | 7-phenyl-thiazolo[4,5-c]pyridin-2-yl)-amide |
| 64 | 3-Methoxymethyl-pyrrolidine-1-carboxylic acid (4- |
| | methoxy-7-phenyl-thiazolo[4,5-c]pyridin-2-yl)-amide |
| 65 | Piperidine-1,4-dicarboxylic acid 4-amide 1-[(4- |
| | methoxy-7-phenyl-thiazolo[4,5-c]pyridin-2-yl)-amide] |
| 66 | 4-Diethylamino-N-(4-methoxy-7-phenyl-thiazolo[4,5- |
| | c]pyridin-2-yl)-benzamide |
| 67 | 4-Difluoromethyl-4-hydroxy-piperidine-1-carboxylic |
| | acid [4-methoxy-7-(1-methyl-1H-pyrazol-4-yl)-thiazolo[4,5- |
| | c]pyridin-2-yl]-amide |
| 68 | 4-Hydroxymethyl-4-methyl-piperidine-1-carboxylic acid |
| | [4-methoxy-7-(1-methyl-1H-pyrazol-4-yl)-thiazolo[4,5- |
| | c]pyridin-2-yl]-amide |
| 69 | 4-Fluoromethyl-4-hydroxy-piperidine-1-carboxylic acid |
| | [4-methoxy-7-(1-methyl-1H-pyrazol-4-yl)-thiazolo[4,5- |
| | c]pyridin-2-yl]-amide |
| 70 | 3-Oxa-9-aza-spiro[5.5]undecane-9-carboxylic acid [4- |
| | methoxy-7-(1-methyl-1H-pyrazol-4-yl)-thiazolo[4,5-c]pyridin-2- |
| | yl]-amide |
| 71 | 4-Methyl-piperidine-1-carboxylic acid [4-methoxy-7-(1- |
| | methyl-1H-pyrazol-4-yl)-thiazolo[4,5-c]pyridin-2-yl]-amide |
| 72 | 4-Benzyl-4-hydroxy-piperidine-1-carboxylic acid [4- |
| | methoxy-7-(1-methyl-1H-pyrazol-4-yl)-thiazolo[4,5-c]pyridin-2- |
| | yl]-amide |
| 73 | 2-Oxo-1-oxa-3,8-diaza-spiro[4.5]decane-8-carboxylic |
| | acid [4-methoxy-7-(1-methyl-1H-pyrazol-4-yl)-thiazolo[4,5- |
| | c]pyridin-2-yl]-amide |
| 74 | 3-Oxo-2,8-diaza-spiro[4.5]decane-8-carboxylic acid [4- |
| | methoxy-7-(1-methyl-1H-pyrazol-4-yl)-thiazolo[4,5-c]pyridin-2- |
| | yl]-amide |
| 75 | 4-Dimethylaminomethyl-N-[4-methoxy-7-(1-methyl-1H- |
| | pyrazol-4-yl)-thiazolo[4,5-c]pyridin-2-yl]-benzamide |
| 76 | 2,4-Dioxo-1,3,8-triaza-spiro[4.5]decane-8-carboxylic |
| | acid [4-methoxy-7-(1-methyl-1H-pyrazol-4-yl)-thiazolo[4,5- |
| | c]pyridin-2-yl]-amide |
| 77 | 4′-Hydroxy-3′,4′,5′,6′-tetrahydro-2′H-[3,4′]bipyridinyl-1′- |
| | carboxylic acid [4-methoxy-7-(1-methyl-1H-pyrazol-4-yl)- |
| | thiazolo[4,5-c]pyridin-2-yl]-amide |
| 78 | 4-Oxo-piperidine-1-carboxylic acid [4-methoxy-7-(1- |
| | methyl-1H-pyrazol-4-yl)-thiazolo[4,5-c]pyridin-2-yl]-amide |
| 79 | 1-Oxo-2,8-diaza-spiro[4.5]decane-8-carboxylic acid [4- |
| | methoxy-7-(1-methyl-1H-pyrazol-4-yl)-thiazolo[4,5-c]pyridin-2- |
| | yl]-amide |
| 80 | 7-Oxa-2-aza-spiro[4.5]decane-2-carboxylic acid [4- |
| | methoxy-7-(1-methyl-1H-pyrazol-4-yl)-thiazolo[4,5-c]pyridin-2- |
| | yl]-amide |
| 81 | 8-Oxa-2-aza-spiro[4.5]decane-2-carboxylic acid [4- |
| | methoxy-7-(1-methyl-1H-pyrazol-4-yl)-thiazolo[4,5-c]pyridin-2- |
| | yl]-amide |
| 82 | 2-Oxa-7-aza-spiro[4.4]nonane-7-carboxylic acid [4- |
| | methoxy-7-(1-methyl-1H-pyrazol-4-yl)-thiazolo[4,5-c]pyridin-2- |
| | yl]-amide |
| 83 | 4-Imidazol-1-ylmethyl-N-[4-methoxy-7-(1-methyl-1H- |
| | pyrazol-4-yl)-thiazolo[4,5-c]pyridin-2-yl]-benzamide |
| 84 | Isoxazole-3-carboxylic acid [4-methoxy-7-(1-methyl- |
| | 1H-pyrazol-4-yl)-thiazolo[4,5-c]pyridin-2-yl]-amide |
| 85 | 4-Hydroxy-4-prop-2-ynyl-piperidine-1-carboxylic acid |
| | [4-methoxy-7-(1-methyl-1H-pyrazol-4-yl)-thiazolo[4,5- |
| | c]pyridin-2-yl]-amide |
| 86 | N-[4-Methoxy-7-(1-methyl-1H-pyrazol-4-yl)- |
| | thiazolo[4,5-c]pyridin-2-yl]-N′,N′-dimethyl-terephthalamide |
| 87 | N-[4-Methoxy-7-(1-methyl-1H-pyrazol-4-yl)- |
| | thiazolo[4,5-c]pyridin-2-yl]-4-trifluoromethoxy-benzamide |
| 88 | 2-Methyl-oxazole-4-carboxylic acid [4-methoxy-7-(1- |
| | methyl-1H-pyrazol-4-yl)-thiazolo[4,5-c]pyridin-2-yl]-amide |
| 89 | Benzooxazole-5-carboxylic acid [4-methoxy-7-(1- |
| | methyl-1H-pyrazol-4-yl)-thiazolo[4,5-c]pyridin-2-yl]-amide |
| 90 | N-[4-Methoxy-7-(1-methyl-1H-pyrazol-4-yl)- |
| | thiazolo[4,5-c]pyridin-2-yl]-4-(2-oxo-pyrrolidin-1-ylmethyl)- |
| | benzamide |
| 91 | 2,3-Dihydro-benzofuran-5-carboxylic acid [4-methoxy- |
| | 7-(1-methyl-1H-pyrazol-4-yl)-thiazolo[4,5-c]pyridin-2-yl]-amide |
| 92 | 3-Methoxymethyl-pyrrolidine-1-carboxylic acid [4- |
| | methoxy-7-(1-methyl-1H-pyrazol-4-yl)-thiazolo[4,5-c]pyridin-2- |
| | yl]-amide |
| 93 | Piperidine-1,4-dicarboxylic acid 4-amide 1-{[4- |
| | methoxy-7-(1-methyl-1H-pyrazol-4-yl)-thiazolo[4,5-c]pyridin-2- |
| | yl]-amide} |
| 94 | 4-Diethylamino-N-[4-methoxy-7-(1-methyl-1H-pyrazol- |
| | 4-yl)-thiazolo[4,5-c]pyridin-2-yl]-benzamide |
| 95 | 4-Difluoromethyl-4-hydroxy-piperidine-1-carboxylic |
| | acid [4-methoxy-7-(tetrahydro-pyran-4-yl)-thiazolo[4,5- |
| | c]pyridin-2-yl]-amide |
| 96 | 4-Hydroxymethyl-4-methyl-piperidine-1-carboxylic acid |
| | [4-methoxy-7-(tetrahydro-pyran-4-yl)-thiazolo[4,5-c]pyridin-2- |
| | yl]-amide |
| 97 | 4-Fluoromethyl-4-hydroxy-piperidine-1-carboxylic acid |
| | [4-methoxy-7-(tetrahydro-pyran-4-yl)-thiazolo[4,5-c]pyridin-2- |
| | yl]-amide |
| 98 | 3-Oxa-9-aza-spiro[5.5]undecane-9-carboxylic acid [4- |
| | methoxy-7-(tetrahydro-pyran-4-yl)-thiazolo[4,5-c]pyridin-2-yl]- |
| | amide |
| 99 | 4-Methyl-piperidine-1-carboxylic acid [4-methoxy-7- |
| | (tetrahydro-pyran-4-yl)-thiazolo[4,5-c]pyridin-2-yl]-amide |
| 100 | 4-Benzyl-4-hydroxy-piperidine-1-carboxylic acid [4- |
| | methoxy-7-(tetrahydro-pyran-4-yl)-thiazolo[4,5-c]pyridin-2-yl]- |
| | amide |
| 101 | 2-Oxo-1-oxa-3,8-diaza-spiro[4.5]decane-8-carboxylic |
| | acid [4-methoxy-7-(tetrahydro-pyran-4-yl)-thiazolo[4,5- |
| | c]pyridin-2-yl]-amide |
| 102 | 3-Oxo-2,8-diaza-spiro[4.5]decane-8-carboxylic acid [4- |
| | methoxy-7-(tetrahydro-pyran-4-yl)-thiazolo[4,5-c]pyridin-2-yl]- |
| | amide |
| 103 | 2,4-Dioxo-1,3,8-triaza-spiro[4.5]decane-8-carboxylic |
| | acid [4-methoxy-7-(tetrahydro-pyran-4-yl)-thiazolo[4,5- |
| | c]pyridin-2-yl]-amide |
| 104 | 4′-Hydroxy-3′,4′,5′,6′-tetrahydro-2′H-[3,4′]bipyridinyl-1′- |
| | carboxylic acid [4-methoxy-7-(tetrahydro-pyran-4-yl)- |
| | thiazolo[4,5-c]pyridin-2-yl]-amide |
| 105 | 4-Oxo-piperidine-1-carboxylic acid [4-methoxy-7- |
| | (tetrahydro-pyran-4-yl)-thiazolo[4,5-c]pyridin-2-yl]-amide |
| 106 | 1-Oxo-2,8-diaza-spiro[4.5]decane-8-carboxylic acid [4- |
| | methoxy-7-(tetrahydro-pyran-4-yl)-thiazolo[4,5-c]pyridin-2-yl]- |
| | amide |
| 107 | 7-Oxa-2-aza-spiro[4.5]decane-2-carboxylic acid [4- |
| | methoxy-7-(tetrahydro-pyran-4-yl)-thiazolo[4,5-c]pyridin-2-yl]- |
| | amide |
| 108 | 8-Oxa-2-aza-spiro[4.5]decane-2-carboxylic acid [4- |
| | methoxy-7-(tetrahydro-pyran-4-yl)-thiazolo[4,5-c]pyridin-2-yl]- |
| | amide |
| 109 | 2-Oxa-7-aza-spiro[4.4]nonane-7-carboxylic acid [4- |
| | methoxy-7-(tetrahydro-pyran-4-yl)-thiazolo[4,5-c]pyridin-2-yl]- |
| | amide |
| 110 | 4-Imidazol-1-ylmethyl-N-[4-methoxy-7-(tetrahydro- |
| | pyran-4-yl)-thiazolo[4,5-c]pyridin-2-yl]-benzamide |
| 111 | Isoxazole-3-carboxylic acid [4-methoxy-7-(tetrahydro- |
| | pyran-4-yl)-thiazolo[4,5-c]pyridin-2-yl]-amide |
| 112 | 4-Hydroxy-4-prop-2-ynyl-piperidine-1-carboxylic acid |
| | [4-methoxy-7-(tetrahydro-pyran-4-yl)-thiazolo[4,5-c]pyridin-2- |
| | yl]-amide |
| 113 | N-[4-Methoxy-7-(tetrahydro-pyran-4-yl)-thiazolo[4,5- |
| | c]pyridin-2-yl]-N′,N′-dimethyl-terephthalamide |
| 114 | N-[4-Methoxy-7-(tetrahydro-pyran-4-yl)-thiazolo[4,5- |
| | c]pyridin-2-yl]-4-trifluoromethoxy-benzamide |
| 115 | 2-Methyl-oxazole-4-carboxylic acid [4-methoxy-7- |
| | (tetrahydro-pyran-4-yl)-thiazolo[4,5-c]pyridin-2-yl]-amide |
| 116 | Benzooxazole-5-carboxylic acid [4-methoxy-7- |
| | (tetrahydro-pyran-4-yl)-thiazolo[4,5-c]pyridin-2-yl]-amide |
| 117 | N-[4-Methoxy-7-(tetrahydro-pyran-4-yl)-thiazolo[4,5- |
| | c]pyridin-2-yl]-4-(2-oxo-pyrrolidin-1-ylmethyl)-benzamide |
| 118 | 2,3-Dihydro-benzofuran-5-carboxylic acid [4-methoxy- |
| | 7-(tetrahydro-pyran-4-yl)-thiazolo[4,5-c]pyridin-2-yl]-amide |
| 119 | 3-Methoxymethyl-pyrrolidine-1-carboxylic acid [4- |
| | methoxy-7-(tetrahydro-pyran-4-yl)-thiazolo[4,5-c]pyridin-2-yl]- |
| | amide |
| 120 | Piperidine-1,4-dicarboxylic acid 4-amide 1-{[4- |
| | methoxy-7-(tetrahydro-pyran-4-yl)-thiazolo[4,5-c]pyridin-2-yl]- |
| | amide} |
| 121 | 4-Diethylamino-N-[4-methoxy-7-(tetrahydro-pyran-4- |
| | yl)-thiazolo[4,5-c]pyridin-2-yl]-benzamide |
| 122 | {1-[4-Methoxy-7-(1-methyl-1H-pyrazol-4-yl)- |
| | thiazolo[4,5-c]pyridin-2-ylcarbamoyl]-piperidin-3-yl}-acetic acid |
| 123 | Pyridine-2,5-dicarboxylic acid 2-dimethylamide 5-{[4- |
| | methoxy-7-(1-methyl-1H-pyrazol-4-yl)-thiazolo[4,5-c]pyridin-2- |
| | yl]-amide} |
| 124 | 1-Methyl-1H-pyrazole-4-carboxylic acid [4-methoxy-7- |
| | (1-methyl-1H-pyrazol-4-yl)-thiazolo[4,5-c]pyridin-2-yl]-amide |
| 125 | 5-Methyl-isoxazole-4-carboxylic acid [4-methoxy-7-(1- |
| | methyl-1H-pyrazol-4-yl)-thiazolo[4,5-c]pyridin-2-yl]-amide |
| 126 | 1-(2-Methoxy-ethyl)-1H-[1,2,3]triazole-4-carboxylic |
| | acid [4-methoxy-7-(1-methyl-1H-pyrazol-4-yl)-thiazolo[4,5- |
| | c]pyridin-2-yl]-amide |
| 127 | 1-Methyl-1H-[1,2,3]triazole-4-carboxylic acid [4- |
| | methoxy-7-(1-methyl-1H-pyrazol-4-yl)-thiazolo[4,5-c]pyridin-2- |
| | yl]-amide |
| 128 | 1-Cyano-cyclopropanecarboxylic acid [4-methoxy-7- |
| | (1-methyl-1H-pyrazol-4-yl)-thiazolo[4,5-c]pyridin-2-yl]-amide |
| 129 | 3-Cyano-N-[4-methoxy-7-(1-methyl-1H-pyrazol-4-yl)- |
| | thiazolo[4,5-c]pyridin-2-yl]-propionamide |
| 130 | 2-Methyl-oxazole-5-carboxylic acid [4-methoxy-7-(1- |
| | methyl-1H-pyrazol-4-yl)-thiazolo[4,5-c]pyridin-2-yl]-amide |
| 131 | 2-Methyl-thiazole-5-carboxylic acid [4-methoxy-7-(1- |
| | methyl-1H-pyrazol-4-yl)-thiazolo[4,5-c]pyridin-2-yl]-amide |
| 132 | 4-Hydroxy-pent-2-ynoic acid [4-methoxy-7-(1-methyl- |
| | 1H-pyrazol-4-yl)-thiazolo[4,5-c]pyridin-2-yl]-amide |
| 133 | (S)-3-Methanesulfonyl-pyrrolidine-1-carboxylic acid [4- |
| | methoxy-7-(1-methyl-1H-pyrazol-4-yl)-thiazolo[4,5-c]pyridin-2- |
| | yl]-amide |
| 134 | (S)-3-Fluoro-pyrrolidine-1-carboxylic acid [4-methoxy- |
| | 7-(1-methyl-1H-pyrazol-4-yl)-thiazolo[4,5-c]pyridin-2-yl]-amide |
| 135 | (S)-3-Cyano-pyrrolidine-1-carboxylic acid [4-methoxy- |
| | 7-(1-methyl-1H-pyrazol-4-yl)-thiazolo[4,5-c]pyridin-2-yl]-amide |
| 136 | (R)-3-Dimethylaminomethyl-pyrrolidine-1-carboxylic |
| | acid [4-methoxy-7-(1-methyl-1H-pyrazol-4-yl)-thiazolo[4,5- |
| | c]pyridin-2-yl]-amide |
| 137 | 5-Methyl-isoxazole-4-carboxylic acid (4-methoxy-7- |
| | morpholin-4-yl-thiazolo[4,5-c]pyridin-2-yl)-amide |
| 138 | 1-(2-Methoxy-ethyl)-1H-[1,2,3]triazole-4-carboxylic |
| | acid (4-methoxy-7-morpholin-4-yl-thiazolo[4,5-c]pyridin-2-yl)- |
| | amide |
| 139 | 1-Methyl-1H-[1,2,3]triazole-4-carboxylic acid (4- |
| | methoxy-7-morpholin-4-yl-thiazolo[4,5-c]pyridin-2-yl)-amide |
| 140 | Pyridine-2,5-dicarboxylic acid 2-dimethylamide 5-{[4- |
| | methoxy-7-(tetrahydro-pyran-4-yl)-thiazolo[4,5-c]pyridin-2-yl]- |
| | amide} |
| 141 | Pyridine-2,5-dicarboxylic acid 2-dimethylamide 5-[(4- |
| | methoxy-7-morpholin-4-yl-thiazolo[4,5-c]pyridin-2-yl)-amide] |
| 142 | 1-Methyl-1H-pyrazole-4-carboxylic acid [7-methoxy-4- |
| | (tetrahydro-pyran-4-yl)-1H-benzoimidazol-2-yl]-amide |
| 143 | 5-Methyl-isoxazole-4-carboxylic acid [4-methoxy-7- |
| | (tetrahydro-pyran-4-yl)-thiazolo[4,5-c]pyridin-2-yl]-amide |
| 144 | 1-(2-Methoxy-ethyl)-1H-[1,2,3]triazole-4-carboxylic |
| | acid [4-methoxy-7-(tetrahydro-pyran-4-yl)-thiazolo[4,5- |
| | c]pyridin-2-yl]-amide |
| 145 | 1-Methyl-1H-[1,2,3]triazole-4-carboxylic acid [4- |
| | methoxy-7-(tetrahydro-pyran-4-yl)-thiazolo[4,5-c]pyridin-2-yl]- |
| | amide |
| 146 | 1-Cyano-cyclopropanecarboxylic acid [4-methoxy-7- |
| | (tetrahydro-pyran-4-yl)-thiazolo[4,5-c]pyridin-2-yl]-amide |
| 147 | 2-Methyl-oxazole-5-carboxylic acid [4-methoxy-7- |
| | (tetrahydro-pyran-4-yl)-thiazolo[4,5-c]pyridin-2-yl]-amide |
| 148 | 2-Methyl-thiazole-5-carboxylic acid [4-methoxy-7- |
| | (tetrahydro-pyran-4-yl)-thiazolo[4,5-c]pyridin-2-yl]-amide |
| 149 | (S)-3-Methanesulfonyl-pyrrolidine-1-carboxylic acid [4- |
| | methoxy-7-(tetrahydro-pyran-4-yl)-thiazolo[4,5-c]pyridin-2-yl]- |
| | amide |
| 150 | (S)-3-Fluoro-pyrrolidine-1-carboxylic acid [4-methoxy- |
| | 7-(tetrahydro-pyran-4-yl)-thiazolo[4,5-c]pyridin-2-yl]-amide |
| 151 | (S)-3-Cyano-pyrrolidine-1-carboxylic acid [4-methoxy- |
| | 7-(tetrahydro-pyran-4-yl)-thiazolo[4,5-c]pyridin-2-yl]-amide |
| 152 | (R)-3-Dimethylaminomethyl-pyrrolidine-1-carboxylic |
| | acid [4-methoxy-7-(tetrahydro-pyran-4-yl)-thiazolo[4,5- |
| | c]pyridin-2-yl]-amide |
| 153 | 1-[4-Methoxy-7-(tetrahydro-pyran-4-yl)-thiazolo[4,5- |
| | c]pyridin-2-yl]-3-thiazol-2-ylmethyl-urea |
| 154 | 1-(2-Methoxy-ethyl)-1H-pyrazole-4-carboxylic acid [4- |
| | methoxy-7-(2-oxa-7-aza-spiro[4.4]non-7-yl)-thiazolo[4,5- |
| | c]pyridin-2-yl]-amide |
| 155 | 1-Methyl-1H-pyrazole-4-carboxylic acid {4-methoxy-7- |
| | [(2-methoxy-propyl)-methyl-amino]-thiazolo[4,5-c]pyridin-2-yl}- |
| | amide |
| 156 | 1-Methyl-1H-pyrazole-4-carboxylic acid [4-methoxy-7- |
| | (5-oxa-2-aza-spiro[3.4]oct-2-yl)-thiazolo[4,5-c]pyridin-2-yl]- |
| | amide |
| 157 | 1-Methyl-1H-pyrazole-4-carboxylic acid (4-methoxy-7- |
| | piperidin-4-yl-thiazolo[4,5-c]pyridin-2-yl)-amide |
| 158 | 1-Methyl-1H-pyrazole-4-carboxylic acid (4-methoxy-7- |
| | piperidin-3-yl-thiazolo[4,5-c]pyridin-2-yl)-amide |
| 159 | 1-Methyl-1H-pyrazole-4-carboxylic acid [7- |
| | (carbamoylmethyl-methyl-amino)-4-methoxy-thiazolo[4,5- |
| | c]pyridin-2-yl]-amide |
| 160 | 1-Methyl-1H-pyrazole-4-carboxylic acid [4-methoxy-7- |
| | (2,2,2-trifluoro-ethoxy)-thiazolo[4,5-c]pyridin-2-yl]-amide |
| 161 | 4-Hydroxy-4-methyl-piperidine-1-carboxylic acid (4- |
| | fluoromethoxy-7-morpholin-4-yl-thiazolo[4,5-c]pyridin-2-yl)- |
| | amide |
| 162 | 4-Hydroxy-4-methyl-piperidine-1-carboxylic acid (4- |
| | difluoromethoxy-7-morpholin-4-yl-thiazolo[4,5-c]pyridin-2-yl)- |
| | amide |
| 163 | N-(4-methoxy-6-methyl-7-morpholino-thiazolo[4,5- |
| | c]pyridin-2-yl)-1-methyl-pyrazole-4-carboxamide |
| 164 | N-(6-bromo-4-methoxy-7-morpholino-thiazolo[4,5- |
| | c]pyridin-2-yl)-1-methyl-pyrazole-4-carboxamide |
| 165 | 1-Methyl-1H-pyrazole-4-carboxylic acid (6-fluoro-4- |
| | methoxy-7-morpholin-4-yl-thiazolo[4,5-c]pyridin-2-yl)-amide |
| 166 | N-(6-chloro-4-methoxy-7-morpholino-thiazolo[4,5- |
| | c]pyridin-2-yl)-1-methyl-pyrazole-4-carboxamide |
| 167 | Cyclopropanecarboxylic acid (4-methoxy-7-morpholin- |
| | 4-yl-thiazolo[4,5-c]pyridin-2-yl)-amide |
| 168 | (S)-7-Oxa-2-aza-spiro[4.5]decane-2-carboxylic acid (4- |
| | methoxy-7-morpholin-4-yl-thiazolo[4,5-c]pyridin-2-yl)-amide |
| 169 | (R)-7-Oxa-2-aza-spiro[4.5]decane-2-carboxylic acid |
| | (4-methoxy-7-morpholin-4-yl-thiazolo[4,5-c]pyridin-2-yl)-amide |
| 170 | N-(4-Methoxy-7-morpholin-4-yl-thiazolo[4,5-c]pyridin- |
| | 2-yl)-4-(2-oxo-pyrrolidin-1-ylmethyl)-benzamide |
| 171 | 1-Methyl-1H-pyrazole-4-carboxylic acid (4-methoxy-7- |
| | morpholin-4-yl-thiazolo[4,5-c]pyridin-2-yl)-amide |
| 172 | (R)-2-Oxa-7-aza-spiro[4.4]nonane-7-carboxylic acid |
| | [4-methoxy-7-(1-methyl-1H-pyrazol-4-yl)-thiazolo[4,5- |
| | c]pyridin-2-yl]-amide |
| 173 | (S)-2-Oxa-7-aza-spiro[4.4]nonane-7-carboxylic acid [4- |
| | methoxy-7-(1-methyl-1H-pyrazol-4-yl)-thiazolo[4,5-c]pyridin-2- |
| | yl]-amide |
| 174 | 3-cyano-N-[4-methoxy-7-(morpholin-4-yl)- |
| | [1,3]thiazolo[4,5-c]pyridin-2-yl]propanamide |
| 175 | 1-cyano-N-[4-methoxy-7-(morpholin-4-yl)- |
| | [1,3]thiazolo[4,5-c]pyridin-2-yl]cyclopropane-1-carboxamide |
| 176 | (S)-7-Oxa-2-aza-spiro[4.5]decane-2-carboxylic acid [4- |
| | methoxy-7-(1-methyl-1H-pyrazol-4-yl)-thiazolo[4,5-c]pyridin-2- |
| | yl]-amide |
| 177 | (R)-7-Oxa-2-aza-spiro[4.5]decane-2-carboxylic acid [4- |
| | methoxy-7-(1-methyl-1H-pyrazol-4-yl)-thiazolo[4,5-c]pyridin-2- |
| | yl]-amide |
| 178 | 1-Methyl-1H-pyrazole-4-carboxylic acid (6-bromo-4- |
| | methoxy-7-morpholin-4-yl-thiazolo[4,5-c]pyridin-2-yl)-amide |
| 179 | Cyclopropanecarboxylic acid [4-methoxy-7-(1-methyl- |
| | 1H-pyrazol-4-yl)-thiazolo[4,5-c]pyridin-2-yl]-amide |
| 180 | N-[4-Methoxy-7-(tetrahydro-pyran-4-yl)-thiazolo[4,5- |
| | c]pyridin-2-yl]-4-(2-oxo-pyrrolidin-1-ylmethyl)-benzamide |
| 181 | 2-Methyl-thiazole-5-carboxylic acid [4-methoxy-7- |
| | (tetrahydro-pyran-4-yl)-thiazolo[4,5-c]pyridin-2-yl]-amide |
| 182 | 2-Oxa-7-aza-spiro[4.4]nonane-7-carboxylic acid [7-(3- |
| | ethoxy-3-methyl-azetidin-1-yl)-4-methoxy-thiazolo[4,5- |
| | c]pyridin-2-yl]-amide |
| 183 | 2-Oxa-7-aza-spiro[4.4]nonane-7-carboxylic acid (4- |
| | methoxy-7-piperidin-4-yl-thiazolo[4,5-c]pyridin-2-yl)-amide |
| 184 | 2-Oxa-7-aza-spiro[4.4]nonane-7-carboxylic acid [4- |
| | methoxy-7-(5-oxa-2-aza-spiro[3.4]oct-2-yl)-thiazolo[4,5- |
| | c]pyridin-2-yl]-amide |
| 185 | 2-Oxa-7-aza-spiro[4.4]nonane-7-carboxylic acid [4- |
| | methoxy-7-(3-methoxy-3-methyl-azetidin-1-yl)-thiazolo[4,5- |
| | c]pyridin-2-yl]-amide |
| 186 | 2-Methyl-oxazole-5-carboxylic acid (4-methoxy-7- |
| | phenyl-thiazolo[4,5-c]pyridin-2-yl)-amide |
| 187 | Cyclopropanecarboxylic acid (4-methoxy-7-phenyl- |
| | thiazolo[4,5-c]pyridin-2-yl)-amide |
| 188 | 1-Methyl-1H-pyrazole-4-carboxylic acid (4-methoxy-7- |
| | phenyl-thiazolo[4,5-c]pyridin-2-yl)-amide |
| 189 | N-[4-methoxy-7-(morpholin-4-yl)-[1,3]thiazolo[4,5- |
| | c]pyridin-2-yl]-2-methyl-1,3-oxazole-5-carboxamide |
| 190 | 2-Methyl-thiazole-5-carboxylic acid (4-methoxy-7- |
| | phenyl-thiazolo[4,5-c]pyridin-2-yl)-amide |
| 191 | 8-Oxa-2-aza-spiro[4.5]decane-2-carboxylic acid [4- |
| | methoxy-7-(5-oxa-2-aza-spiro[3.4]oct-2-yl)-thiazolo[4,5- |
| | c]pyridin-2-yl]-amide |
| 192 | 8-Oxa-2-aza-spiro[4.5]decane-2-carboxylic acid (4- |
| | methoxy-7-piperidin-4-yl-thiazolo[4,5-c]pyridin-2-yl)-amide |
| 193 | 8-Oxa-2-aza-spiro[4.5]decane-2-carboxylic acid [7-(4- |
| | fluoro-phenyl)-4-methoxy-thiazolo[4,5-c]pyridin-2-yl]-amide |
| 194 | 8-Oxa-2-aza-spiro[4.5]decane-2-carboxylic acid [7-(3- |
| | fluoro-phenyl)-4-methoxy-thiazolo[4,5-c]pyridin-2-yl]-amide |
| 195 | 8-Oxa-2-aza-spiro[4.5]decane-2-carboxylic acid [7-(2- |
| | fluoro-phenyl)-4-methoxy-thiazolo[4,5-c]pyridin-2-yl]-amide |
| 196 | 1-Dimethylaminomethyl-cyclopropanecarboxylic acid |
| | (4-methoxy-7-phenyl-thiazolo[4,5-c]pyridin-2-yl)-amide |
| 197 | Cyclopropane-1,1-dicarboxylic acid dimethylamide (4- |
| | methoxy-7-phenyl-thiazolo[4,5-c]pyridin-2-yl)-amide |
| 198 | 1-Imidazol-1-ylmethyl-cyclopropanecarboxylic acid (4- |
| | methoxy-7-phenyl-thiazolo[4,5-c]pyridin-2-yl)-amide |
| 199 | 1-Methyl-1H-pyrazole-4-carboxylic acid (4-hydroxy-7- |
| | morpholin-4-yl-thiazolo[4,5-c]pyridin-2-yl)-amide |
| 200 | 8-Oxa-2-aza-spiro[4.5]decane-2-carboxylic acid (4- |
| | methoxy-7-piperidin-3-yl-thiazolo[4,5-c]pyridin-2-yl)-amide |
| 201 | 1-amino-N-{4-methoxy-7-phenyl-[1,3]thiazolo[4,5- |
| | c]pyridin-2-yl}-8-azaspiro[4.5]decane-8-carboxamide |
| 202 | (1S,2S)-2-methoxy-N-{4-methoxy-7-phenyl- |
| | [1,3]thiazolo[4,5-c]pyridin-2-yl}cyclopropane-1-carboxamide |
| 203 | 1-Amino-cyclopropanecarboxylic acid (4-methoxy-7- |
| | phenyl-thiazolo[4,5-c]pyridin-2-yl)-amide |
| 204 | (S)-7-Oxa-2-aza-spiro[4.5]decane-2-carboxylic acid (4- |
| | methoxy-7-phenyl-thiazolo[4,5-c]pyridin-2-yl)-amide |
| 205 | (R)-7-Oxa-2-aza-spiro[4.5]decane-2-carboxylic acid |
| | (4-methoxy-7-phenyl-thiazolo[4,5-c]pyridin-2-yl)-amide |
| 206 | 8-Oxa-2-aza-spiro[4.5]decane-2-carboxylic acid (4- |
| | methoxy-7-pyridin-3-yl-thiazolo[4,5-c]pyridin-2-yl)-amide |
| 207 | (S)-2-Oxa-7-aza-spiro[4.4]nonane-7-carboxylic acid |
| | (4-methoxy-7-phenyl-thiazolo[4,5-c]pyridin-2-yl)-amide |
| 208 | (R)-2-Oxa-7-aza-spiro[4.4]nonane-7-carboxylic acid |
| | (4-methoxy-7-phenyl-thiazolo[4,5-c]pyridin-2-yl)-amide |
| 209 | 8-Oxa-2-aza-spiro[4.5]decane-2-carboxylic acid [7- |
| | (2,3-difluoro-phenyl)-4-methoxy-thiazolo[4,5-c]pyridin-2-yl]- |
| | amide |
| 210 | 8-Oxa-2-aza-spiro[4.5]decane-2-carboxylic acid [7- |
| | (2,5-difluoro-phenyl)-4-methoxy-thiazolo[4,5-c]pyridin-2-yl]- |
| | amide |
| 211 | 8-Oxa-2-aza-spiro[4.5]decane-2-carboxylic acid (4- |
| | methoxy-7-pyridin-2-yl-thiazolo[4,5-c]pyridin-2-yl)-amide |
| 212 | 8-Oxa-2-aza-spiro[4.5]decane-2-carboxylic acid (4- |
| | methoxy-7-pyridin-4-yl-thiazolo[4,5-c]pyridin-2-yl)-amide |
| 213 | 4-(2,5-Dioxo-pyrrolidin-1-yl)-N-(4-methoxy-7-phenyl- |
| | thiazolo[4,5-c]pyridin-2-yl)-benzamide |
| 214 | 4-(2,5-Dioxo-pyrrolidin-1-yl)-N-(4-methoxy-7- |
| | morpholin-4-yl-thiazolo[4,5-c]pyridin-2-yl)-benzamide |
| 215 | (R)-1-Amino-8-aza-spiro[4.5]decane-8-carboxylic acid |
| | (4-methoxy-7-phenyl-thiazolo[4,5-c]pyridin-2-yl)-amide |
| 216 | (S)-1-Amino-8-aza-spiro[4.5]decane-8-carboxylic acid |
| | (4-methoxy-7-phenyl-thiazolo[4,5-c]pyridin-2-yl)-amide |
| 217 | 1-Methyl-1H-pyrazole-4-carboxylic acid (6-cyano-4- |
| | methoxy-7-morpholin-4-yl-thiazolo[4,5-c]pyridin-2-yl)-amide |
| 218 | N-(4-Methoxy-7-pyridin-3-yl-thiazolo[4,5-c]pyridin-2- |
| | yl)-N′,N′-dimethyl-terephthalamide |
| 219 | N-(4-Methoxy-7-pyridin-4-yl-thiazolo[4,5-c]pyridin-2- |
| | yl)-N′,N′-dimethyl-terephthalamide |
| 220 | 8-Oxa-2-aza-spiro[4.5]decane-2-carboxylic acid (6- |
| | chloro-4-methoxy-7-phenyl-thiazolo[4,5-c]pyridin-2-yl)-amide |
| 221 | 1-Methyl-1H-pyrazole-4-carboxylic acid (6-chloro-4- |
| | methoxy-7-morpholin-4-yl-thiazolo[4,5-c]pyridin-2-yl)-amide |
| 222 | 8-Oxa-2-aza-spiro[4.5]decane-2-carboxylic acid (6- |
| | cyano-4-methoxy-7-phenyl-thiazolo[4,5-c]pyridin-2-yl)-amide |
| 223 | 8-Oxa-2-aza-spiro[4.5]decane-2-carboxylic acid (4- |
| | methoxy-6-methyl-7-phenyl-thiazolo[4,5-c]pyridin-2-yl)-amide |
| 224 | N-{4-Methoxy-7-[3-(2-methoxy-ethoxy)-phenyl]- |
| | thiazolo[4,5-c]pyridin-2-yl}-N′,N′-dimethyl-terephthalamide |
| 225 | 8-Oxa-2-aza-spiro[4.5]decane-2-carboxylic acid {4- |
| | methoxy-7-[3-(2-methoxy-ethoxy)-phenyl]-thiazolo[4,5- |
| | c]pyridin-2-yl}-amide |
| 226 | 4-(2,5-Dioxo-pyrrolidin-1-yl)-N-(6-fluoro-4-methoxy-7- |
| | morpholin-4-yl-thiazolo[4,5-c]pyridin-2-yl)-benzamide |
| 227 | N-(2-Hydroxy-ethyl)-N′-(4-methoxy-7-phenyl- |
| | thiazolo[4,5-c]pyridin-2-yl)-N-methyl-terephthalamide |
| 228 | N-(2-Hydroxy-ethyl)-N′-(4-methoxy-7-phenyl- |
| | thiazolo[4,5-c]pyridin-2-yl)-terephthalamide |
| 229 | 8-Oxa-2-aza-spiro[4.5]decane-2-carboxylic acid (6- |
| | fluoro-4-methoxy-7-phenyl-thiazolo[4,5-c]pyridin-2-yl)-amide |
| 230 | N-(2-Dimethylamino-ethyl)-N′-(4-methoxy-7-phenyl- |
| | thiazolo[4,5-c]pyridin-2-yl)-terephthalamide |
| 231 | 8-Oxa-2-aza-spiro[4.5]decane-2-carboxylic acid (4,6- |
| | dimethyl-7-phenyl-thiazolo[4,5-c]pyridin-2-yl)-amide |
| 232 | 8-Oxa-2-aza-spiro[4.5]decane-2-carboxylic acid [4- |
| | methoxy-7-(tetrahydro-pyran-4-yl)-thiazolo[4,5-c]pyridin-2-yl]- |
| | amide |
| 233 | N-{4-methoxy-7-[3-(2-methoxyethoxy)phenyl]- |
| | [1,3]thiazolo[4,5-c]pyridin-2-yl}-1-(2-methoxyethyl)-1H- |
| | pyrazole-4-carboxamide |
| 234 | N-{6-fluoro-4-methoxy-7-[3-(2-methoxyethoxy)phenyl]- |
| | [1,3]thiazolo[4,5-c]pyridin-2-yl}-8-oxa-2-azaspiro[4.5]decane- |
| | 2-carboxamide |
| 235 | N-[4-methoxy-7-(oxan-4-yl)-[1,3]thiazolo[4,5-c]pyridin- |
| | 2-yl]-1-methyl-1H-pyrazole-4-carboxamide |
| 236 | N-[4-methoxy-7-(2-methoxyphenyl)-[1,3]thiazolo[4,5- |
| | c]pyridin-2-yl]-8-oxa-2-azaspiro[4.5]decane-2-carboxamide |
| 237 | 8-Oxa-2-aza-spiro[4.5]decane-2-carboxylic acid [7- |
| | (3,6-dihydro-2H-pyran-4-yl)-4-methoxy-thiazolo[4,5-c]pyridin- |
| | 2-yl]-amide |
| 238 | N-[7-(3,6-dihydro-2H-pyran-4-yl)-4-methoxy- |
| | [1,3]thiazolo[4,5-c]pyridin-2-yl]-1-methyl-1H-pyrazole-4- |
| | carboxamide |
| 239 | N-[7-(2,6-dimethoxypyridin-3-yl)-4-methoxy- |
| | [1,3]thiazolo[4,5-c]pyridin-2-yl]-8-oxa-2-azaspiro[4.5]decane- |
| | 2-carboxamide |
| 240 | 8-Oxa-2-aza-spiro[4.5]decane-2-carboxylic acid (6- |
| | fluoro-4-hydroxy-7-phenyl-thiazolo[4,5-c]pyridin-2-yl)-amide |
| 241 | 2-amino-N-[4-methoxy-7-(oxan-4-yl)-[1,3]thiazolo[4,5- |
| | c]pyridin-2-yl]-1,3-thiazole-5-carboxamide |
| 242 | 3-Amino-pyrrolidine-1-carboxylic acid (4-methoxy-7- |
| | phenyl-thiazolo[4,5-c]pyridin-2-yl)-amide |
| 243 | (R)-3-Amino-pyrrolidine-1-carboxylic acid (4-methoxy- |
| | 7-phenyl-thiazolo[4,5-c]pyridin-2-yl)-amide |
| 244 | (S)-3-Amino-pyrrolidine-1-carboxylic acid (4-methoxy- |
| | 7-phenyl-thiazolo[4,5-c]pyridin-2-yl)-amide |
| 245 | 8-Oxa-2-aza-spiro[4.5]decane-2-carboxylic acid (7- |
| | phenyl-thiazolo[4,5-c]pyridin-2-yl)-amide |
| 246 | 1-(2-Methoxy-ethyl)-1H-pyrazole-4-carboxylic acid (6- |
| | fluoro-4-methoxy-7-morpholin-4-yl-thiazolo[4,5-c]pyridin-2-yl)- |
| | amide |
| 247 | Bicyclo[1.1.1]pentane-1,3-dicarboxylic acid |
| | dimethylamide (4-methoxy-7-morpholin-4-yl-thiazolo[4,5- |
| | c]pyridin-2-yl)-amide |
| 248 | N-(2-Hydroxy-ethyl)-N′-[4-methoxy-7-(tetrahydro- |
| | pyran-4-yl)-thiazolo[4,5-c]pyridin-2-yl]-N-methyl- |
| | terephthalamide |

and pharmaceutically acceptable salts, derivatives, solvates, prodrugs and stereoisomers thereof, including mixtures thereof in all ratios.

TABLE 2

| | |
|---|---|
| 1 | (R)-3-Aminomethyl-pyrrolidine-1-carboxylic acid (4- |
| | methoxy-7-phenyl-thiazolo[4,5-c]pyridin-2-yl)-amide |
| 2 | N-{4-methoxy-7-[4-(oxan-4-yloxy)phenyl]- |
| | [1,3]thiazolo[4,5-c]pyridin-2-yl}-8-oxa-2-azaspiro[4.5]decane- |
| | 2-carboxamide |
| 3 | (S)-3-Aminomethyl-pyrrolidine-1-carboxylic acid (4- |
| | methoxy-7-phenyl-thiazolo[4,5-c]pyridin-2-yl)-amide |
| 4 | Cyclopropanecarboxylic acid (6-fluoro-4-methoxy-7- |
| | morpholin-4-yl-thiazolo[4,5-c]pyridin-2-yl)-amide |
| 5 | 4-Methoxy-7-(tetrahydro-pyran-4-yl)-thiazolo[4,5- |
| | c]pyridin-2-ylamine |
| 6 | N-(6-Fluoro-4-methoxy-7-morpholin-4-yl-thiazolo[4,5- |
| | c]pyridin-2-yl)-4-(1H-tetrazol-5-yl)-benzamide |
| 7 | 7-Oxa-2-aza-spiro[4.5]decane-2-carboxylic acid (6- |
| | fluoro-4-methoxy-7-morpholin-4-yl-thiazolo[4,5-c]pyridin-2-yl)- |
| | amide |
| 8 | 7-(3,6-Dihydro-2H-pyran-4-yl)-4-methoxy-thiazolo[4,5- |
| | c]pyridin-2-ylamine |
| 9 | N-[7-(1H-indol-6-yl)-4-methoxy-[1,3]thiazolo[4,5- |
| | c]pyridin-2-yl]-8-oxa-2-azaspiro[4.5]decane-2-carboxamide |
| 10 | (R)-7-Oxa-2-aza-spiro[4.5]decane-2-carboxylic acid |
| | (6-fluoro-4-methoxy-7-morpholin-4-yl-thiazolo[4,5-c]pyridin-2- |
| | yl)-amide |
| 11 | (5S)-N-[6-fluoro-4-methoxy-7-(morpholin-4-yl)- |
| | [1,3]thiazolo[4,5-c]pyridin-2-yl]-7-oxa-2-azaspiro[4.5]decane- |
| | 2-carboxamide |
| 12 | (R)-7-Oxa-2-aza-spiro[4.5]decane-2-carboxylic acid |
| | (6-fluoro-4-methoxy-7-phenyl-thiazolo[4,5-c]pyridin-2-yl)- |
| | amide |
| 13 | (5S)-N-{6-fluoro-4-methoxy-7-phenyl-[1,3]thiazolo[4,5- |
| | c]pyridin-2-yl}-7-oxa-2-azaspiro[4.5]decane-2-carboxamide |
| 14 | 3-Dimethylaminomethyl-bicyclo[1.1.1]pentane-1- |
| | carboxylic acid (4-methoxy-7-morpholin-4-yl-thiazolo[4,5- |
| | c]pyridin-2-yl)-amide |
| 15 | 7-Oxa-2-aza-spiro[4.5]decane-2-carboxylic acid [7- |
| | (3,6-dihydro-2H-pyran-4-yl)-4-methoxy-thiazolo[4,5-c]pyridin- |
| | 2-yl]-amide |
| 16 | N-[6-fluoro-4-methoxy-7-(morpholin-4-yl)- |
| | [1,3]thiazolo[4,5-c]pyridin-2-yl]-2-oxa-7-azaspiro[4.4]nonane- |
| | 7-carboxamide |
| 17 | N-[4-methoxy-7-(oxan-4-yl)-[1,3]thiazolo[4,5-c]pyridin- |
| | 2-yl]-2-[(2-methoxyethyl)amino]-1,3-thiazole-5-carboxamide |
| 18 | (R)-2-Oxa-7-aza-spiro[4.4]nonane-7-carboxylic acid |
| | (6-fluoro-4-methoxy-7-morpholin-4-yl-thiazolo[4,5-c]pyridin-2- |
| | yl)-amide |
| 19 | (5S)-N-[6-fluoro-4-methoxy-7-(morpholin-4-yl)- |
| | [1,3]thiazolo[4,5-c]pyridin-2-yl]-2-oxa-7-azaspiro[4.4]nonane- |
| | 7-carboxamide |
| 20 | N-[6-Fluoro-4-methoxy-7-(tetrahydro-pyran-4-yl)- |
| | thiazolo[4,5-c]pyridin-2-yl]-N′,N′-dimethyl-terephthalamide |
| 21 | 1-Imidazol-1-ylmethyl-cyclopropanecarboxylic acid [6- |
| | fluoro-4-methoxy-7-(tetrahydro-pyran-4-yl)-thiazolo[4,5- |
| | c]pyridin-2-yl]-amide |
| 22 | N-[6-fluoro-4-methoxy-7-(oxan-4-yl)-[1,3]thiazolo[4,5- |
| | c]pyridin-2-yl]-1-(2-methoxyethyl)-1H-pyrazole-4-carboxamide |
| 23 | N-[6-fluoro-4-methoxy-7-(oxan-4-yl)-[1,3]thiazolo[4,5- |
| | c]pyridin-2-yl]-1-methyl-1H-pyrazole-4-carboxamide |
| 24 | (R)-7-Oxa-2-aza-spiro[4.5]decane-2-carboxylic acid [7- |
| | (3,6-dihydro-2H-pyran-4-yl)-4-methoxy-thiazolo[4,5-c]pyridin- |
| | 2-yl]-amide |
| 25 | (S)-7-Oxa-2-aza-spiro[4.5]decane-2-carboxylic acid [7- |
| | (3,6-dihydro-2H-pyran-4-yl)-4-methoxy-thiazolo[4,5-c]pyridin- |
| | 2-yl]-amide |
| 26 | 8-Oxa-2-aza-spiro[4.5]decane-2-carboxylic acid [6- |
| | fluoro-4-methoxy-7-(tetrahydro-pyran-4-yl)-thiazolo[4,5- |
| | c]pyridin-2-yl]-amide |
| 27 | 4-Hydroxy-4-methyl-piperidine-1-carboxylic acid [6- |
| | fluoro-7-(4-fluoro-phenyl)-4-methoxy-thiazolo[4,5-c]pyridin-2- |
| | yl]-amide |
| 28 | Cyclopropanecarboxylic acid [6-fluoro-4-methoxy-7- |
| | (tetrahydro-pyran-4-yl)-thiazolo[4,5-c]pyridin-2-yl]-amide |
| 29 | 8-Oxa-2-aza-spiro[4.5]decane-2-carboxylic acid [6- |
| | fluoro-7-(4-fluoro-phenyl)-4-methoxy-thiazolo[4,5-c]pyridin-2- |
| | yl]-amide |
| 30 | Cyclopropanecarboxylic acid [7-(3-ethylaminomethyl- |
| | phenyl)-4-methoxy-thiazolo[4,5-c]pyridin-2-yl]-amide |
| 31 | 7-Oxa-2-aza-spiro[4.5]decane-2-carboxylic acid [6- |
| | fluoro-4-methoxy-7-(tetrahydro-pyran-4-yl)-thiazolo[4,5- |
| | c]pyridin-2-yl]-amide |
| 32 | 1H-Imidazole-4-carboxylic acid (6-fluoro-4-methoxy-7- |
| | phenyl-thiazolo[4,5-c]pyridin-2-yl)-amide |
| 33 | N-[6-fluoro-4-methoxy-7-(oxan-4-yl)-[1,3]thiazolo[4,5- |
| | c]pyridin-2-yl]-2-oxa-7-azaspiro[4.4]nonane-7-carboxamide |
| 34 | (R)-7-Oxa-2-aza-spiro[4.5]decane-2-carboxylic acid [6- |
| | fluoro-4-methoxy-7-(tetrahydro-pyran-4-yl)-thiazolo[4,5- |
| | c]pyridin-2-yl]-amide |
| 35 | (5S)-N-[6-fluoro-4-methoxy-7-(oxan-4-yl)- |
| | [1,3]thiazolo[4,5-c]pyridin-2-yl]-7-oxa-2-azaspiro[4.5]decane- |
| | 2-carboxamide |
| 36 | 1-Methyl-1H-pyrazole-4-carboxylic acid [4-methoxy-7- |
| | (2,2,2-trifluoro-ethoxy)-thiazolo[4,5-c]pyridin-2-yl]-amide |
| 37 | (R)-2-Oxa-7-aza-spiro[4.4]nonane-7-carboxylic acid |
| | [6-fluoro-4-methoxy-7-(tetrahydro-pyran-4-yl)-thiazolo[4,5- |
| | c]pyridin-2-yl]-amide |
| 38 | (5S)-N-[6-fluoro-4-methoxy-7-(oxan-4-yl)- |
| | [1,3]thiazolo[4,5-c]pyridin-2-yl]-2-oxa-7-azaspiro[4.4]nonane- |
| | 7-carboxamide |
| 39 | 8-Oxa-2-aza-spiro[4.5]decane-2-carboxylic acid [7-(3- |
| | amino-phenyl)-4-methoxy-thiazolo[4,5-c]pyridin-2-yl]-amide |
| 40 | 8-Oxa-2-aza-spiro[4.5]decane-2-carboxylic acid [4- |
| | methoxy-7-(3-oxo-cyclopent-1-enyl)-thiazolo[4,5-c]pyridin-2- |
| | yl]-amide |
| 41 | Bicyclo[1.1.1]pentane-1,3-dicarboxylic acid [6-fluoro-4- |
| | methoxy-7-(tetrahydro-pyran-4-yl)-thiazolo[4,5-c]pyridin-2-yl]- |
| | amide (2-hydroxy-ethyl)-methyl-amide |
| 42 | N-[7-(2,5-dihydrofuran-3-yl)-4-methoxy- |
| | [1,3]thiazolo[4,5-c]pyridin-2-yl]cyclopropanecarboxamide |
| 43 | N-[7-(2,5-dihydrofuran-3-yl)-4-methoxy- |
| | [1,3]thiazolo[4,5-c]pyridin-2-yl]-1H-imidazole-4-carboxamide |
| 44 | N-[7-(2,5-dihydrofuran-3-yl)-4-methoxy- |
| | [1,3]thiazolo[4,5-c]pyridin-2-yl]-8-oxa-2-azaspiro[4.5]decane- |
| | 2-carboxamide |
| 45 | N-[7-(2,5-dihydrofuran-3-yl)-4-methoxy- |
| | [1,3]thiazolo[4,5-c]pyridin-2-yl]-7-oxa-2-azaspiro[4.5]decane- |
| | 2-carboxamide |
| 46 | 8-Oxa-2-aza-spiro[4.5]decane-2-carboxylic acid [7-(1- |
| | acetyl-1,2,3,6-tetrahydro-pyridin-4-yl)-4-methoxy-thiazolo[4,5- |
| | c]pyridin-2-yl]-amide |
| 47 | 8-Oxa-2-aza-spiro[4.5]decane-2-carboxylic acid (4- |
| | methoxy-7-thiophen-2-yl-thiazolo[4,5-c]pyridin-2-yl)-amide |
| 48 | 8-Oxa-2-aza-spiro[4.5]decane-2-carboxylic acid (7- |
| | furan-2-yl-4-methoxy-thiazolo[4,5-c]pyridin-2-yl)-amide |
| 49 | 8-Oxa-2-aza-spiro[4.5]decane-2-carboxylic acid [7-(3- |
| | ethylaminomethyl-phenyl)-4-methoxy-thiazolo[4,5-c]pyridin-2- |
| | yl]-amide |
| 50 | N-[6-Fluoro-4-methoxy-7-(tetrahydro-pyran-4-yl)- |
| | thiazolo[4,5-c]pyridin-2-yl]-N′-(2-hydroxy-ethyl)-N′-methyl- |
| | terephthalamide |
| 51 | 8-Oxa-2-aza-spiro[4.5]decane-2-carboxylic acid (4- |
| | methoxy-7-piperidin-1-yl-thiazolo[4,5-c]pyridin-2-yl)-amide |
| 52 | 8-Oxa-2-aza-spiro[4.5]decane-2-carboxylic acid (7- |
| | furan-3-yl-4-methoxy-thiazolo[4,5-c]pyridin-2-yl)-amide |
| 53 | 8-Oxa-2-aza-spiro[4.5]decane-2-carboxylic acid [4- |
| | methoxy-7-(4-methyl-piperazin-1-yl)-thiazolo[4,5-c]pyridin-2- |
| | yl]-amide |
| 54 | 8-Oxa-2-aza-spiro[4.5]decane-2-carboxylic acid [4- |
| | methoxy-7-(3-methoxy-phenyl)-thiazolo[4,5-c]pyridin-2-yl]- |
| | amide |
| 55 | N-{6-cyano-4-methoxy-[1,3]thiazolo[4,5-c]pyridin-2-yl}- |
| | 1-(2-methoxyethyl)-1H-pyrazole-4-carboxamide |
| 56 | 8-Oxa-2-aza-spiro[4.5]decane-2-carboxylic acid [4- |
| | methoxy-7-(6-methyl-pyridazin-3-yl)-thiazolo[4,5-c]pyridin-2- |
| | yl]-amide |
| 57 | 8-Oxa-2-aza-spiro[4.5]decane-2-carboxylic acid (7- |
| | azetidin-1-yl-4-methoxy-thiazolo[4,5-c]pyridin-2-yl)-amide |
| 58 | 8-Oxa-2-aza-spiro[4.5]decane-2-carboxylic acid [7-(3- |
| | hydroxy-azetidin-1-yl)-4-methoxy-thiazolo[4,5-c]pyridin-2-yl]- |
| | amide |
| 59 | 8-Oxa-2-aza-spiro[4.5]decane-2-carboxylic acid (7- |
| | cyclohex-1-enyl-4-methoxy-thiazolo[4,5-c]pyridin-2-yl)-amide |
| 60 | 1H-Imidazole-4-carboxylic acid (4-methoxy-7-phenyl- |
| | thiazolo[4,5-c]pyridin-2-yl)-amide |
| 61 | N4-[7-(3,6-dihydro-2H-pyran-4-yl)-4-methoxy- |
| | [1,3]thiazolo[4,5-c]pyridin-2-yl]-N1,N1-dimethylbenzene-1,4- |
| | dicarboxamide |
| 62 | 8-Oxa-2-aza-spiro[4.5]decane-2-carboxylic acid (7- |
| | cyclohexyl-4-methoxy-thiazolo[4,5-c]pyridin-2-yl)-amide |
| 63 | 8-Oxa-2-aza-spiro[4.5]decane-2-carboxylic acid [7- |
| | (4,4-difluoro-cyclohex-1-enyl)-4-methoxy-thiazolo[4,5- |
| | c]pyridin-2-yl]-amide |
| 64 | 8-Oxa-2-aza-spiro[4.5]decane-2-carboxylic acid [7- |
| | (3,6-dihydro-2H-thiopyran-4-yl)-4-methoxy-thiazolo[4,5- |
| | c]pyridin-2-yl]-amide |
| 65 | 1H-Imidazole-4-carboxylic acid [7-(3,6-dihydro-2H- |
| | pyran-4-yl)-4-methoxy-thiazolo[4,5-c]pyridin-2-yl]-amide |
| 66 | N-[4-methoxy-7-(4-methoxycyclohex-1-en-1-yl)- |
| | [1,3]thiazolo[4,5-c]pyridin-2-yl]-8-oxa-2-azaspiro[4.5]decane- |
| | 2-carboxamide |
| 67 | 8-Oxa-2-aza-spiro[4.5]decane-2-carboxylic acid [4- |
| | methoxy-7-(2-methyl-thiazol-4-yl)-thiazolo[4,5-c]pyridin-2-yl]- |
| | amide |
| 68 | 8-Oxa-2-aza-spiro[4.5]decane-2-carboxylic acid [4- |
| | methoxy-7-(1-pyridin-3-ylmethyl-1H-pyrazol-4-yl)-thiazolo[4,5- |
| | c]pyridin-2-yl]-amide |
| 69 | 8-Oxa-2-aza-spiro[4.5]decane-2-carboxylic acid [4- |
| | methoxy-7-(1-pyridin-2-ylmethyl-1H-pyrazol-4-yl)-thiazolo[4,5- |
| | c]pyridin-2-yl]-amide |
| 70 | (5R)-N-[4-methoxy-7-(4-methoxycyclohex-1-en-1-yl)- |
| | [1,3]thiazolo[4,5-c]pyridin-2-yl]-7-oxa-2-azaspiro[4.5]decane- |
| | 2-carboxamide |
| 71 | N-[7-(3,6-dihydro-2H-pyran-4-yl)-4-methoxy- |
| | [1,3]thiazolo[4,5-c]pyridin-2-yl]-4-(1H-1,2,3-triazol-1- |
| | yl)benzamide |
| 72 | 4-{[7-(3,6-dihydro-2H-pyran-4-yl)-4-methoxy- |
| | [1,3]thiazolo[4,5-c]pyridin-2-yl]carbamoyl}benzoic acid |
| 73 | 8-Oxa-2-aza-spiro[4.5]decane-2-carboxylic acid (7- |
| | [1,4]dioxan-2-yl-4-methoxy-thiazolo[4,5-c]pyridin-2-yl)-amide |
| 74 | 8-Oxa-2-aza-spiro[4.5]decane-2-carboxylic acid {7-[1- |
| | (2,2-difluoro-ethyl)-1H-pyrazol-4-yl]-4-methoxy-thiazolo[4,5- |
| | c]pyridin-2-yl}-amide |
| 75 | 8-Oxa-2-aza-spiro[4.5]decane-2-carboxylic acid [4- |
| | methoxy-7-(1-pyridin-4-ylmethyl-1H-pyrazol-4-yl)-thiazolo[4,5- |
| | c]pyridin-2-yl]-amide |
| 76 | 8-Oxa-2-aza-spiro[4.5]decane-2-carboxylic acid [7-(1- |
| | benzyl-1H-pyrazol-4-yl)-4-methoxy-thiazolo[4,5-c]pyridin-2-yl]- |
| | amide |
| 77 | (5S)-N-[4-methoxy-7-(4-methoxycyclohex-1-en-1-yl)- |
| | [1,3]thiazolo[4,5-c]pyridin-2-yl]-7-oxa-2-azaspiro[4.5]decane- |
| | 2-carboxamide |
| 78 | 8-Oxa-2-aza-spiro[4.5]decane-2-carboxylic acid [4- |
| | methoxy-7-(6-oxo-1,6-dihydro-pyridin-3-yl)-thiazolo[4,5- |
| | c]pyridin-2-yl]-amide |
| 79 | 8-Oxa-2-aza-spiro[4.5]decane-2-carboxylic acid [7-(1- |
| | difluoromethyl-1H-pyrazol-4-yl)-4-methoxy-thiazolo[4,5- |
| | c]pyridin-2-yl]-amide |
| 80 | 8-Oxa-2-aza-spiro[4.5]decane-2-carboxylic acid (4- |
| | difluoromethoxy-7-phenyl-thiazolo[4,5-c]pyridin-2-yl)-amide |
| 81 | N-[7-(3,6-dihydro-2H-pyran-4-yl)-4-methoxy- |
| | [1,3]thiazolo[4,5-c]pyridin-2-yl]-2-[(2-methoxyethyl)amino]-1,3- |
| | thiazole-5-carboxamide |
| 82 | N-[7-(3,6-dihydro-2H-pyran-4-yl)-4-methoxy- |
| | [1,3]thiazolo[4,5-c]pyridin-2-yl]-4-[(1H-imidazol-1- |
| | yl)methyl]benzamide |
| 83 | N-[7-(3,6-dihydro-2H-pyran-4-yl)-4-methoxy- |
| | [1,3]thiazolo[4,5-c]pyridin-2-yl]-4-[(1R)-1- |
| | acetamidoethyl]benzamide |
| 84 | 8-Oxa-2-aza-spiro[4.5]decane-2-carboxylic acid {4- |
| | methoxy-7-[1-(tetrahydro-pyran-2-ylmethyl)-1H-pyrazol-4-yl]- |
| | thiazolo[4,5-c]pyridin-2-yl}-amide |
| 85 | 8-Oxa-2-aza-spiro[4.5]decane-2-carboxylic acid {4- |
| | methoxy-7-[1-(tetrahydro-pyran-4-ylmethyl)-1H-pyrazol-4-yl]- |
| | thiazolo[4,5-c]pyridin-2-yl}-amide |
| 86 | 8-Oxa-2-aza-spiro[4.5]decane-2-carboxylic acid [7- |
| | (1,1-dioxo-hexahydro-1I6-thiopyran-4-yl)-4-methoxy- |
| | thiazolo[4,5-c]pyridin-2-yl]-amide |
| 87 | 8-Oxa-2-aza-spiro[4.5]decane-2-carboxylic acid {4- |
| | methoxy-7-[1-(tetrahydro-pyran-3-ylmethyl)-1H-pyrazol-4-yl]- |
| | thiazolo[4,5-c]pyridin-2-yl}-amide |
| 88 | N-[7-(3,6-dihydro-2H-pyran-4-yl)-4-methoxy- |
| | [1,3]thiazolo[4,5-c]pyridin-2-yl]-4-(2,5-dioxo-2,5-dihydro-1H- |
| | pyrrol-1-yl)piperidine-1-carboxamide |
| 89 | 3-[7-(3,6-dihydro-2H-pyran-4-yl)-4-methoxy- |
| | [1,3]thiazolo[4,5-c]pyridin-2-yl]-1-[4-(2-oxopyrrolidin-1- |
| | yl)phenyl]urea |
| 90 | N-[4-({[7-(3,6-dihydro-2H-pyran-4-yl)-4-methoxy- |
| | [1,3]thiazolo[4,5-c]pyridin-2-yl]carbamoyl}amino)phenyl]-2- |
| | (dimethylamino)acetamide |
| 91 | N-[7-(3,6-dihydro-2H-pyran-4-yl)-4-methoxy- |
| | [1,3]thiazolo[4,5-c]pyridin-2-yl]-4-(2,4-dioxo-1,3-thiazolidin-3- |
| | yl)piperidine-1-carboxamide |
| 92 | N-[4-({[7-(3,6-dihydro-2H-pyran-4-yl)-4-methoxy- |
| | [1,3]thiazolo[4,5-c]pyridin-2-yl]carbamoyl}amino)-2- |
| | methylphenyl]acetamide |
| 93 | N4-[7-(3,6-dihydro-2H-pyran-4-yl)-4-hydroxy- |
| | [1,3]thiazolo[4,5-c]pyridin-2-yl]-N1-(2-hydroxyethyl)-N1- |
| | methylbenzene-1,4-dicarboxamide |
| 94 | 3-[7-(3,6-dihydro-2H-pyran-4-yl)-4-methoxy- |
| | [1,3]thiazolo[4,5-c]pyridin-2-yl]-1-[4-(3-methyl-5-oxo-4,5- |
| | dihydro-1H-pyrazol-1-yl)phenyl]urea |
| 95 | 3-[7-(3,6-dihydro-2H-pyran-4-yl)-4-methoxy- |
| | [1,3]thiazolo[4,5-c]pyridin-2-yl]-1-[4-(2-oxo-1,3-oxazolidin-3- |
| | yl)phenyl]urea |
| 96 | N1-[7-(3,6-dihydro-2H-pyran-4-yl)-4-methoxy- |
| | [1,3]thiazolo[4,5-c]pyridin-2-yl]-N4,N4-dimethylpiperidine-1,4- |
| | dicarboxamide |
| 97 | [4-(4-Methoxy-7-phenyl-thiazolo[4,5-c]pyridin-2- |
| | ylcarbamoyl)-benzyl]-methyl-carbamic acid methyl ester |
| 98 | 2,8-Diaza-spiro[4.5]decane-2-carboxylic acid [7-(3,6- |
| | dihydro-2H-pyran-4-yl)-4-methoxy-thiazolo[4,5-c]pyridin-2-yl]- |
| | amide |
| 99 | 4-(2,5-Dioxo-pyrrolidin-1-yl)-piperidine-1-carboxylic |
| | acid (4-methoxy-7-phenyl-thiazolo[4,5-c]pyridin-2-yl)-amide |
| 100 | 4-(2,5-Dioxo-pyrrolidin-1-yl)-piperidine-1-carboxylic |
| | acid [7-(3,6-dihydro-2H-pyran-4-yl)-4-methoxy-thiazolo[4,5- |
| | c]pyridin-2-yl]-amide |
| 101 | Bicyclo[1.1.1]pentane-1,3-dicarboxylic acid (6-fluoro-4- |
| | methoxy-7-phenyl-thiazolo[4,5-c]pyridin-2-yl)-amide (2- |
| | hydroxy-ethyl)-methyl-amide |
| 102 | 2,7-Diaza-spiro[4.5]decane-2-carboxylic acid [7-(3,6- |
| | dihydro-2H-pyran-4-yl)-4-methoxy-thiazolo[4,5-c]pyridin-2-yl]- |
| | amide |
| 103 | 8-Oxa-2-aza-spiro[4.5]decane-2-carboxylic acid (4- |
| | methoxy-7-{1-[2-(2-methoxy-ethoxy)-ethyl]-1H-pyrazol-4-yl}- |
| | thiazolo[4,5-c]pyridin-2-yl)-amide |
| 104 | 8-Oxa-2-aza-spiro[4.5]decane-2-carboxylic acid (4- |
| | methoxy-7-{1-[(R)-1-(tetrahydro-pyran-3-yl)methyl]-1H- |
| | pyrazol-4-yl}-thiazolo[4,5-c]pyridin-2-yl)-amide |
| 105 | 8-Oxa-2-aza-spiro[4.5]decane-2-carboxylic acid (4- |
| | methoxy-7-{1-[(S)-1-(tetrahydro-pyran-3-yl)methyl]-1H- |
| | pyrazol-4-yl}-thiazolo[4,5-c]pyridin-2-yl)-amide |
| 106 | N1-[7-(3,6-dihydro-2H-pyran-4-yl)-4-methoxy- |
| | [1,3]thiazolo[4,5-c]pyridin-2-yl]piperidine-1,4-dicarboxamide |
| 107 | N-[7-(3,6-dihydro-2H-pyran-4-yl)-4-hydroxy- |
| | [1,3]thiazolo[4,5-c]pyridin-2-yl]-2-oxa-7-azaspiro[4.4]nonane- |
| | 7-carboxamide |
| 108 | N-[7-(3,6-dihydro-2H-pyran-4-yl)-4-methoxy- |
| | [1,3]thiazolo[4,5-c]pyridin-2-yl]-2-oxa-7-azaspiro[4.4]nonane- |
| | 7-carboxamide |
| 109 | 4-({4-methoxy-7-phenyl-[1,3]thiazolo[4,5-c]pyridin-2- |
| | yl}carbamoyl)benzoic acid |
| 110 | N-{4-methoxy-7-phenyl-[1,3]thiazolo[4,5-c]pyridin-2- |
| | yl}-4-(1H-1,2,3,4-tetrazol-5-yl)benzamide |
| 111 | 8-Oxa-2-aza-spiro[4.5]decane-2-carboxylic acid [7- |
| | (4,4-difluoro-cyclohexyl)-4-methoxy-thiazolo[4,5-c]pyridin-2- |
| | yl]-amide |
| 112 | 8-Oxa-2-aza-spiro[4.5]decane-2-carboxylic acid [4- |
| | methoxy-7-(3-methylamino-phenyl)-thiazolo[4,5-c]pyridin-2- |
| | yl]-amide |
| 113 | 8-Oxa-2-aza-spiro[4.5]decane-2-carboxylic acid [4- |
| | methoxy-7-(5-methyl-thiophen-2-yl)-thiazolo[4,5-c]pyridin-2- |
| | yl]-amide |
| 114 | 8-Oxa-2-aza-spiro[4.5]decane-2-carboxylic acid [4- |
| | methoxy-7-(5-methyl-furan-2-yl)-thiazolo[4,5-c]pyridin-2-yl]- |
| | amide |
| 115 | 4-[(4-methoxy-7-{1-[(pyridin-3-yl)methyl]-1H-pyrazol-4- |
| | yl}-[1,3]thiazolo[4,5-c]pyridin-2-yl)carbamoyl]benzoic acid |
| 116 | N-[7-(3,6-dihydro-2H-pyran-4-yl)-4-methoxy- |
| | [1,3]thiazolo[4,5-c]pyridin-2-yl]-1H-pyrazole-4-carboxamide |
| 117 | N-{4-methoxy-7-phenyl-[1,3]thiazolo[4,5-c]pyridin-2- |
| | yl}-1H-pyrazole-4-carboxamide |
| 118 | 8-Oxa-2-aza-spiro[4.5]decane-2-carboxylic acid (4- |
| | methoxy-7-{1-[(S)-1-(tetrahydro-pyran-2-yl)methyl]-1H- |
| | pyrazol-4-yl}-thiazolo[4,5-c]pyridin-2-yl)-amide |
| 119 | 8-Oxa-2-aza-spiro[4.5]decane-2-carboxylic acid (4- |
| | methoxy-7-{1-[(R)-1-(tetrahydro-pyran-2-yl)methyl]-1H- |
| | pyrazol-4-yl}-thiazolo[4,5-c]pyridin-2-yl)-amide |
| 120 | 8-Oxa-2-aza-spiro[4.5]decane-2-carboxylic acid [7-(3- |
| | methanesulfonylamino-phenyl)-4-methoxy-thiazolo[4,5- |
| | c]pyridin-2-yl]-amide |
| 121 | (R)-2,7-Diaza-spiro[4.5]decane-2-carboxylic acid [7- |
| | (3,6-dihydro-2H-pyran-4-yl)-4-methoxy-thiazolo[4,5-c]pyridin- |
| | 2-yl]-amide |
| 122 | (S)-2,7-Diaza-spiro[4.5]decane-2-carboxylic acid [7- |
| | (3,6-dihydro-2H-pyran-4-yl)-4-methoxy-thiazolo[4,5-c]pyridin- |
| | 2-yl]-amide |
| 123 | Piperidine-1,4-dicarboxylic acid 4-dimethylamide 1-[(4- |
| | methoxy-7-phenyl-thiazolo[4,5-c]pyridin-2-yl)-amide] |
| 124 | 8-Oxa-2-aza-spiro[4.5]decane-2-carboxylic acid [7-(2- |
| | amino-pyridin-4-yl)-4-methoxy-thiazolo[4,5-c]pyridin-2-yl]- |
| | amide |
| 125 | N-[7-(3,6-Dihydro-2H-pyran-4-yl)-4-methoxy- |
| | thiazolo[4,5-c]pyridin-2-yl]-4-(4-methyl-piperazine-1-carbonyl)- |
| | benzamide |
| 126 | N-[7-(3,6-Dihydro-2H-pyran-4-yl)-4-methoxy- |
| | thiazolo[4,5-c]pyridin-2-yl]-N′-(2-piperidin-1-yl-ethyl)- |
| | terephthalamide |
| 127 | 8-Oxa-2-aza-spiro[4.5]decane-2-carboxylic acid [4- |
| | methoxy-7-(2-methylamino-pyridin-4-yl)-thiazolo[4,5-c]pyridin- |
| | 2-yl]-amide |
| 128 | 8-Oxa-2-aza-spiro[4.5]decane-2-carboxylic acid [4- |
| | methoxy-7-(5-methyl-cyclohex-1-enyl)-thiazolo[4,5-c]pyridin-2- |
| | yl]-amide |
| 129 | N-[7-(3,6-Dihydro-2H-pyran-4-yl)-4-methoxy- |
| | thiazolo[4,5-c]pyridin-2-yl]-4-(4-hydroxy-4-methyl-piperidine-1- |
| | carbonyl)-benzamide |
| 130 | 8-Oxa-2-aza-spiro[4.5]decane-2-carboxylic acid [7-(3- |
| | fluoro-5-methanesulfonylamino-phenyl)-4-methoxy- |
| | thiazolo[4,5-c]pyridin-2-yl]-amide |
| 131 | 4-(2,5-Dioxo-imidazolidin-1-yl)-piperidine-1-carboxylic |
| | acid [7-(3,6-dihydro-2H-pyran-4-yl)-4-methoxy-thiazolo[4,5- |
| | c]pyridin-2-yl]-amide |
| 132 | 8-Oxa-2-aza-spiro[4.5]decane-2-carboxylic acid [4- |
| | methoxy-7-(3-methyl-3,6-dihydro-2H-pyran-4-yl)-thiazolo[4,5- |
| | c]pyridin-2-yl]-amide |
| 133 | 8-Oxa-2-aza-spiro[4.5]decane-2-carboxylic acid [4- |
| | methoxy-7-(3-trifluoromethyl-piperidin-1-yl)-thiazolo[4,5- |
| | c]pyridin-2-yl]-amide |
| 134 | 8-Oxa-2-aza-spiro[4.5]decane-2-carboxylic acid [4- |
| | methoxy-7-(3-methoxy-piperidin-1-yl)-thiazolo[4,5-c]pyridin-2- |
| | yl]-amide |
| 135 | Imidazo[1,2-a]pyridine-3-carboxylic acid [7-(3,6- |
| | dihydro-2H-pyran-4-yl)-4-methoxy-thiazolo[4,5-c]pyridin-2-yl]- |
| | amide |
| 136 | 8-Oxa-2-aza-spiro[4.5]decane-2-carboxylic acid [4- |
| | methoxy-7-(5-oxo-2,5-dihydro-1H-pyrrol-3-yl)-thiazolo[4,5- |
| | c]pyridin-2-yl]-amide |
| 137 | 4-(2,5-Dioxo-imidazolidin-1-yl)-piperidine-1-carboxylic |
| | acid (4-methoxy-7-phenyl-thiazolo[4,5-c]pyridin-2-yl)-amide |
| 138 | 8-Oxa-2-aza-spiro[4.5]decane-2-carboxylic acid [7-(5- |
| | amino-2-fluoro-pyridin-3-yl)-4-methoxy-thiazolo[4,5-c]pyridin- |
| | 2-yl]-amide |
| 139 | N-(2-Azetidin-1-yl-ethyl)-N′-[7-(3,6-dihydro-2H-pyran- |
| | 4-yl)-4-methoxy-thiazolo[4,5-c]pyridin-2-yl]-terephthalamide |
| 140 | 2-Pyridin-3-yl-1H-imidazole-4-carboxylic acid [7-(3,6- |
| | dihydro-2H-pyran-4-yl)-4-methoxy-thiazolo[4,5-c]pyridin-2-yl]- |
| | amide |
| 141 | N-{4-methoxy-7-[3-(trifluoromethyl)phenyl]- |
| | [1,3]thiazolo[4,5-c]pyridin-2-yl}-8-oxa-2-azaspiro[4.5]decane- |
| | 2-carboxamide |
| 142 | 8-Oxa-2-aza-spiro[4.5]decane-2-carboxylic acid [7-(5- |
| | amino-6-fluoro-pyridin-3-yl)-4-methoxy-thiazolo[4,5-c]pyridin- |
| | 2-yl]-amide |
| 143 | 8-Oxa-2-aza-spiro[4.5]decane-2-carboxylic acid [7-(5- |
| | amino-pyridin-3-yl)-4-methoxy-thiazolo[4,5-c]pyridin-2-yl]- |
| | amide |
| 144 | {4-[7-(3,6-Dihydro-2H-pyran-4-yl)-4-methoxy- |
| | thiazolo[4,5-c]pyridin-2-ylcarbamoyl]-phenyl}-acetic acid |
| 145 | 8-Oxa-2-aza-spiro[4.5]decane-2-carboxylic acid [4- |
| | methoxy-7-((S)-3-methyl-cyclohex-1-enyl)-thiazolo[4,5- |
| | c]pyridin-2-yl]-amide |
| 146 | 8-Oxa-2-aza-spiro[4.5]decane-2-carboxylic acid [4- |
| | methoxy-7-((R)-3-methyl-cyclohex-1-enyl)-thiazolo[4,5- |
| | c]pyridin-2-yl]-amide |

and pharmaceutically acceptable salts, derivatives, solvates, prodrugs and stereoisomers thereof, including mixtures thereof in all ratios.

In one embodiment, the therapeutic combination of the invention is used in the treatment of a human subject. The main expected benefit in the treatment with the therapeutic combination is a gain in risk/benefit ratio for these human patients. The administration of the combinations of the invention may be advantageous over the individual therapeutic agents in that the combinations may provide one or more of the following improved properties when compared to the individual administration of a single therapeutic agent alone: i) a greater anticancer effect than the most active single agent, ii) synergistic or highly synergistic anticancer activity, iii) a dosing protocol that provides enhanced anticancer activity with reduced side effect profile, iv) a reduction in the toxic effect profile, v) an increase in the therapeutic window, and/or vi) an increase in the bioavailability of one or both of the therapeutic agents.

In certain embodiments, the invention provides for the treatment of diseases, disorders, and conditions characterized by excessive or abnormal cell proliferation. Such diseases include a proliferative or hyperproliferative disease. Examples of proliferative and hyperproliferative diseases include cancer and myeloproliferative disorders.

In another embodiment, the cancer is selected from carcinoma, lymphoma, leukemia, blastoma, and sarcoma. More particular examples of such cancers include squamous cell carcinoma, myeloma, small-cell lung cancer, non-small cell lung cancer, glioma, Hodgkin's lymphoma, non-Hodgkin's lymphoma, acute myeloid leukemia, multiple myeloma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma, cervical cancer, brain cancer, stomach cancer, bladder cancer, hepatoma, breast cancer, colon carcinoma, biliary tract cancer, and head and neck cancer. The disease or medical disorder in question may be selected from any of those disclosed in WO2015118175, WO2018029367, WO2018208720, PCT/US18/12604, PCT/US19/47734, PCT/US19/40129, PCT/US19/36725, PCT/US19/732271, PCT/US19/38600, PCT/EP2019/061558. In some embodiments, the cancer is selected from lung adenocarcinoma, head-neck squamous cell carcinoma, esophageal carcinoma, stomach adenocarcinoma, pancreatic adenocarcinoma, rectum adenocarcinoma and colon adenocarcinoma.

In some embodiments, the cancer is a CD73-expressing cancer. In some embodiments, the cancer is a cancer with elevated adenosine levels, e.g., extracellular adenosine levels, in the tumor microenvironment. In some embodiments, the cancer has an adenosine gene-expression signature reflecting increased levels of adenosine, which may be measured, for instance, in peripheral blood or tumor samples. Such gene-expression signatures include the ones outlined in Fong et al. 2019, Cancer Discov. 10:40-53; DiRenzo et al. 2019, Abstract 3168, Cancer Res. 79:3168 and in Sidders et al., Clin. Cancer Res. 26, 2176-2187 (2020). In some embodiments, the adenosine gene-expression signature comprises evaluating the expression of CD73 and/or tissue non-specific alkaline phosphatase (TNAP). In some embodiments, the adenosine gene-expression signature comprises evaluating the expression of one or more of CXCL1, CXCL2, CXCL3, CXCL5, CXCL6, CXCL8, IL1p and PTGS2, which may be measured, for instance, in peripheral blood mononuclear cells (PBMC). In some embodiments, the adenosine gene-expression signature comprises evaluating the expression of one or more of PPARG, CYBB, COL3A1, FOXP3, LAG3, APP, CD81, GPI, PTGS2, CASP1, FOS, MAPK1, MAPK3, and CREB1. In some embodiments, the adenosine gene-expression signature comprises evaluating the expression of one or more enzymes of the adenosine signaling pathway, such as CD39, CD73, the adenosine A_{2A} receptor and the adenosine A_{2B} receptor.

In some embodiments, the cancer is a cancer with adenosine A_{2B} receptor-mediated signaling.

In various embodiments, the method of the invention is employed as a first, second, third or later line of treatment. A line of treatment refers to a place in the order of treatment with different medications or other therapies received by a patient. First line therapy regimens are treatments given first, whereas second or third line therapy is given after the first line therapy or after the second line therapy, respectively. Therefore, first line therapy is the first treatment for a disease or condition. In patients with cancer, first line therapy, sometimes referred to as primary therapy or primary treatment, can be surgery, chemotherapy, radiation therapy, or a combination of these therapies. Typically, a patient is given a subsequent chemotherapy regimen (second or third line therapy), either because the patient did not show a positive clinical outcome or only showed a sub-clinical response to a first or second line therapy or showed a positive clinical response but later experienced a relapse, sometimes with disease now resistant to the earlier therapy that elicited the earlier positive response.

In some embodiments, the therapeutic combination of the invention is applied in a later line of treatment, particularly a second line or higher treatment of the cancer. There is no limitation to the prior number of therapies provided that the subject underwent at least one round of prior cancer therapy. The round of prior cancer therapy refers to a defined schedule/phase for treating a subject with, e.g., one or more chemotherapeutic agents, radiotherapy or chemoradiotherapy, and the subject failed with such previous treatment, which was either completed or terminated ahead of schedule. One reason could be that the cancer was resistant or became resistant to prior therapy. The current standard of care (SoC) for treating cancer patients often involves the administration of toxic and old chemotherapy regimens. Such SoC is associated with high risks of strong adverse events that are likely to interfere with the quality of life (such as secondary cancers). In one embodiment, the combined administration of the PD-1 inhibitor, TGFβ inhibitor, and adenosine inhibitor may be as effective and better tolerated than the SoC in patients with cancer. As the modes of action of the PD-1 inhibitor, TGFβ inhibitor, and adenosine inhibitor are different, it is thought that the likelihood that administration of the therapeutic treatment of the invention may lead to enhanced immune-related adverse events (irAE) is small.

In one embodiment, the PD-1 inhibitor, TGFβ inhibitor, and adenosine inhibitor are administered in a second line or higher treatment of a cancer selected from the group of pre-treated relapsing metastatic NSCLC, unresectable locally advanced NSCLC, pre-treated SCLC ED, SCLC unsuitable for systemic treatment, pre-treated relapsing (recurrent) or metastatic SCCHN, recurrent SCCHN eligible for re-irradiation, and pre-treated microsatellite status instable low (MSI-L) or microsatellite status stable (MSS) metastatic colorectal cancer (mCRC). SCLC and SCCHN are particularly systemically pre-treated. MSI-L/MSS mCRC occurs in 85% of all mCRC.

In one embodiment, the cancer exhibits microsatellite instability (MSI). Microsatellite instability (“MSI”) is or comprises a change that in the DNA of certain cells (such as tumor cells) in which the number of repeats of microsatellites (short, repeated sequences of DNA) is different than the number of repeats that was contained in the DNA from which it was inherited. Microsatellite instability arises from a failure to repair replication-associated errors due to a defective DNA mismatch repair (MMR) system. This failure allows persistence of mismatch mutations all over the genome, but especially in regions of repetitive DNA known as microsatellites, leading to increased mutational load. It has been demonstrated that at least some tumors characterized by MSI-H have improved responses to certain anti-PD-1 agents (Le et al. (2015) N. Engl. J. Med. 372(26):2509-2520; Westdorp et al. (2016) Cancer Immunol. Immunother. 65(10): 1249-1259).

In some embodiments, a cancer has a microsatellite instability status of high microsatellite instability (e.g. MSI-H status). In some embodiments, a cancer has a microsatellite instability status of low microsatellite instability (e.g. MSI-L status). In some embodiments, a cancer has a microsatellite instability status of microsatellite stable (e.g. MSS status). In some embodiments microsatellite instability status is assessed by a next generation sequencing (NGS)-based assay, an immunohistochemistry (IHC)-based assay, and/or a PCR-based assay. In some embodiments, microsatellite instability is detected by NGS. In some embodiments, microsatellite instability is detected by IHC. In some embodiments, microsatellite instability is detected by PCR.

In some embodiments, the cancer is associated with a high tumor mutation burden (TMB). In some embodiments, the cancer is associated with high TMB and MSI-H. In some embodiments, the cancer is associated with high TMB and MSI-L or MSS. In some embodiments, the cancer is endometrial cancer associated with high TMB. In some related embodiments, the endometrial cancer is associated with high TMB and MSI-H. In some related embodiments, the endometrial cancer is associated with high TMB and MSI-L or MSS.

In some embodiments, a cancer is a mismatch repair deficient (dMMR) cancer. Microsatellite instability may arise from a failure to repair replication-associated errors due to a defective DNA mismatch repair (MMR) system. This failure allows persistence of mismatch mutations all over the genome, but especially in regions of repetitive DNA known as microsatellites, leading to increased mutational load that may improve responses to certain therapeutic agents.

In some embodiments, a cancer is a hypermutated cancer. In some embodiments, a cancer harbors a mutation in polymerase epsilon (POLE). In some embodiments, a cancer harbors a mutation in polymerase delta (POLD).

In some embodiments, a cancer is endometrial cancer (e.g. MSI-H or MSS/MSI-L endometrial cancer). In some embodiments, a cancer is a MSI-H cancer comprising a mutation in POLE or POLD (e.g. a MSI-H non-endometrial cancer comprising a mutation in POLE or POLD).

In some embodiments, the cancer is an advanced cancer. In some embodiments, the cancer is a metastatic cancer. In some embodiments, the cancer is a recurrent cancer (e.g. a recurrent gynecological cancer such as recurrent epithelial ovarian cancer, recurrent fallopian tube cancer, recurrent primary peritoneal cancer, or recurrent endometrial cancer). In one embodiment, the cancer is recurrent or advanced.

In one embodiment, the cancer is selected from: appendiceal cancer, bladder cancer, breast cancer, cervical cancer, colorectal cancer, endometrial cancer, esophageal cancer (in particular esophageal squamous cell carcinoma), fallopian tube cancer, gastric cancer, glioma (such as diffuse intrinsic pontine glioma), head and neck cancer (in particular head and neck squamous cell carcinoma and oropharyngeal cancer), leukemia (in particular acute lymphoblastic leukemia, acute myeloid leukemia) lung cancer (in particular non small cell lung cancer), lymphoma (in particular Hodgkin's lymphoma, non-Hodgkin's lymphoma), melanoma, mesothelioma (in particular malignant pleural mesothelioma), Merkel cell carcinoma, neuroblastoma, oral cancer, osteosarcoma, ovarian cancer, prostate cancer, renal cancer, salivary gland tumor, sarcoma (in particular Ewing's sarcoma or rhabdomyosarcoma) squamous cell carcinoma, soft tissue sarcoma, thymoma, thyroid cancer, urothelial cancer, uterine cancer, vaginal cancer, vulvar cancer or Wilms tumor. In a further embodiment, the cancer is selected from: appendiceal cancer, bladder cancer, cervical cancer, colorectal cancer, esophageal cancer, head and neck cancer, melanoma, mesothelioma, non-small-cell lung cancer, prostate cancer and urothelial cancer. In a further embodiment, the cancer is selected from cervical cancer, endometrial cancer, head and neck cancer (in particular head and neck squamous cell carcinoma and oropharyngeal cancer), lung cancer (in particular non small cell lung cancer), lymphoma (in particular non-Hodgkin's lymphoma), melanoma, oral cancer, thyroid cancer, urothelial cancer or uterine cancer. In another embodiment, the cancer is selected from head and neck cancer (in particular head and neck squamous cell carcinoma and oropharyngeal cancer), lung cancer (in particular non small cell lung cancer), urothelial cancer, melanoma or cervical cancer.

In one embodiment, the human has a solid tumor. In one embodiment, the solid tumor is advanced solid tumor. In one embodiment, the cancer is selected from head and neck cancer, squamous cell carcinoma of the head and neck (SCCHN or HNSCC), gastric cancer, melanoma, renal cell carcinoma (RCC), esophageal cancer, non-small cell lung carcinoma, prostate cancer, colorectal cancer, ovarian cancer and pancreatic cancer. In one embodiment, the cancer is selected from the group consisting of: colorectal cancer, cervical cancer, bladder cancer, urothelial cancer, head and neck cancer, melanoma, mesothelioma, non-small cell lung carcinoma, prostate cancer, esophageal cancer, and esophageal squamous cell carcinoma. In one aspect the human has one or more of the following: SCCHN, colorectal cancer, esophageal cancer, cervical cancer, bladder cancer, breast cancer, head and neck cancer, ovarian cancer, melanoma, renal cell carcinoma (RCC), esophageal squamous cell carcinoma, non-small cell lung carcinoma, mesothelioma (e.g. pleural malignant mesothelioma), and prostate cancer.

In another aspect the human has a liquid tumor such as diffuse large B cell lymphoma (DLBCL), multiple myeloma, chronic lymphoblastic leukemia, follicular lymphoma, acute myeloid leukemia and chronic myelogenous leukemia.

In one embodiment, the cancer is head and neck cancer. In one embodiment, the cancer is HNSCC. Squamous cell carcinoma is a cancer that arises from particular cells called squamous cells. Squamous cells are found in the outer layer of skin and in the mucous membranes, which are the moist tissues that line body cavities such as the airways and intestines. Head and neck squamous cell carcinoma (HNSCC) develops in the mucous membranes of the mouth, nose, and throat. HNSCC is also known as SCCHN and squamous cell carcinoma of the head and neck.

HNSCC can occur in the mouth (oral cavity), the middle part of the throat near the mouth (oropharynx), the space behind the nose (nasal cavity and paranasal sinuses), the upper part of the throat near the nasal cavity (nasopharynx), the voicebox (larynx), or the lower part of the throat near the larynx (hypopharynx). Depending on the location, the cancer can cause abnormal patches or open sores (ulcers) in the mouth and throat, unusual bleeding or pain in the mouth, sinus congestion that does not clear, sore throat, earache, pain when swallowing or difficulty swallowing, a hoarse voice, difficulty breathing, or enlarged lymph nodes.

HNSCC can metastasize to other parts of the body, such as the lymph nodes, lungs or liver.

Tobacco use and alcohol consumption are the two most important risk factors for the development of HNSCC, and their contributions to risk are synergistic. In addition, the human papillomavirus (HPV), especially HPV-16, is now a well-established independent risk factor. Patients with HNSCC have a relatively poor prognosis. Recurrent/metastatic (R/M) HNSCC is especially challenging, regardless of human papillomavirus (HPV) status, and currently, few effective treatment options are available in the art. HPV-negative HNSCC is associated with a locoregional relapse rate of 19-35% and a distant metastatic rate of 14-22% following standard of care, compared with rates of 9-18% and 5-12%, respectively, for HPV-positive HNSCC. The median overall survival for patients with R/M disease is 10-13 months in the setting of first line chemotherapy and 6 months in the second line setting. The current standard of care is platinum-based doublet chemotherapy with or without cetuximab. Second line standard of care options include cetuximab, methotrexate, and taxanes. All of these chemotherapeutic agents are associated with significant side effects, and only 10-13% of patients respond to treatment. HNSCC regressions from existing systemic therapies are transient and do not add significantly increased longevity, and virtually all patients succumb to their malignancy.

In one embodiment, the cancer is head and neck cancer. In one embodiment the cancer is head and neck squamous cell carcinoma (HNSCC). In one embodiment, the cancer is recurrent/metastatic (R/M) HNSCC. In one embodiment, the cancer is recurring/refractory (R/R) HNSCC. In one embodiment, the cancer is HPV-negative or HPV-positive HNSCC. In one embodiment, the cancer is a locally advanced HNSCC. In one embodiment, the cancer is HNSCC, such as (R/M) HNSCC, in PD-L1 positive patients having a CPS of ≥1% or a TPS ≥50%. The CPS or TPS is as determined by an FDA- or EMA-approved test, such as the Dako IHC 22C3 PharmDx assay. In one embodiment, the cancer is HNSCC in PD-1 inhibitor experienced or PD-1 inhibitor naïve patients. In one embodiment, the cancer is HNSCC in PD-1 inhibitor experienced or PD-1 inhibitor naïve patients.

In one embodiment, the head and neck cancer is oropharyngeal cancer. In one embodiment, the head and neck cancer is an oral cancer (i.e. a mouth cancer).

In one embodiment, the cancer is lung cancer. In some embodiments, the lung cancer is a squamous cell carcinoma of the lung. In some embodiments, the lung cancer is small cell lung cancer (SCLC). In some embodiments, the lung cancer is non-small cell lung cancer (NSCLC), such as squamous NSCLC. In some embodiments, the lung cancer is an ALK-translocated lung cancer (e.g. ALK-translocated NSCLC). In some embodiments, the cancer is NSCLC with an identified ALK translocation. In some embodiments, the lung cancer is an EGFR-mutant lung cancer (e.g. EGFR-mutant NSCLC). In some embodiments, the cancer is NSCLC with an identified EGFR mutation. In one embodiment, the cancer is NSCLC in PD-L1 positive patients having a TPS 1% or a TPS ≥50%. The TPS is as determined by an FDA- or EMA-approved test, such as the Dako IHC 22C3 PharmDx assay or the VENTANA PD-L1 (SP263) assay.

In one embodiment, the cancer is melanoma. In some embodiments, the melanoma is an advanced melanoma. In some embodiments, the melanoma is a metastatic melanoma. In some embodiments, the melanoma is a MSI-H melanoma. In some embodiments, the melanoma is a MSS melanoma. In some embodiments, the melanoma is a POLE-mutant melanoma. In some embodiments, the melanoma is a POLD-mutant melanoma. In some embodiments, the melanoma is a high TMB melanoma.

In one embodiment, the cancer is colorectal cancer. In some embodiments, the colorectal cancer is an advanced colorectal cancer. In some embodiments, the colorectal cancer is a metastatic colorectal cancer. In some embodiments, the colorectal cancer is a MSI-H colorectal cancer. In some embodiments, the colorectal cancer is a MSS colorectal cancer. In some embodiments, the colorectal cancer is a POLE-mutant colorectal cancer. In some embodiments, the colorectal cancer is a POLD-mutant colorectal cancer. In some embodiments, the colorectal cancer is a high TMB colorectal cancer.

In some embodiments, the cancer is a gynecologic cancer (i.e. a cancer of the female reproductive system such as ovarian cancer, fallopian tube cancer, cervical cancer, vaginal cancer, vulvar cancer, uterine cancer, or primary peritoneal cancer, or breast cancer). In some embodiments, cancers of the female reproductive system include, but are not limited to, ovarian cancer, cancer of the fallopian tube(s), peritoneal cancer, and breast cancer.

In some embodiments, the cancer is ovarian cancer (e.g. serous or clear cell ovarian cancer). In some embodiments, the cancer is fallopian tube cancer (e.g. serous or clear cell fallopian tube cancer). In some embodiments, the cancer is primary peritoneal cancer (e.g. serous or clear cell primary peritoneal cancer).

In some embodiments, the ovarian cancer is an epithelial carcinoma. Epithelial carcinomas make up 85% to 90% of ovarian cancers. While historically considered to start on the surface of the ovary, new evidence suggests at least some ovarian cancer begins in special cells in a part of the fallopian tube. The fallopian tubes are small ducts that link a woman's ovaries to her uterus that are a part of a woman's reproductive system. In a normal female reproductive system, there are two fallopian tubes, one located on each side of the uterus. Cancer cells that begin in the fallopian tube may go to the surface of the ovary early on. The term “ovarian cancer” is often used to describe epithelial cancers that begin in the ovary, in the fallopian tube, and from the lining of the abdominal cavity, call the peritoneum. In some embodiments, the cancer is or comprises a germ cell tumor. Germ cell tumors are a type of ovarian cancer develops in the egg-producing cells of the ovaries. In some embodiments, a cancer is or comprises a stromal tumor. Stromal tumors develop in the connective tissue cells that hold the ovaries together, which sometimes is the tissue that makes female hormones called estrogen. In some embodiments, the cancer is or comprises a granulosa cell tumor. Granulosa cell tumors may secrete estrogen resulting in unusual vaginal bleeding at the time of diagnosis. In some embodiments, a gynecologic cancer is associated with homologous recombination repair deficiency/homologous repair deficiency (HRD) and/or BRCA1/2 mutation(s). In some embodiments, a gynecologic cancer is platinum-sensitive. In some embodiments, a gynecologic cancer has responded to a platinum-based therapy. In some embodiments, a gynecologic cancer has developed resistance to a platinum-based therapy. In some embodiments, a gynecologic cancer has at one time shown a partial or complete response to platinum-based therapy (e.g. a partial or complete response to the last platinum-based therapy or to the penultimate platinum-based therapy). In some embodiments, a gynecologic cancer is now resistant to platinum-based therapy.

In some embodiments, the cancer is breast cancer. Usually breast cancer either begins in the cells of the milk producing glands, known as the lobules, or in the ducts. Less commonly breast cancer can begin in the stromal tissues. These include the fatty and fibrous connective tissues of the breast. Over time the breast cancer cells can invade nearby tissues such the underarm lymph nodes or the lungs in a process known as metastasis. The stage of a breast cancer, the size of the tumor and its rate of growth are all factors which determine the type of treatment that is offered. Treatment options include surgery to remove the tumor, drug treatment which includes chemotherapy and hormonal therapy, radiation therapy and immunotherapy. The prognosis and survival rate varies widely; the five year relative survival rates vary from 98% to 23% depending on the type of breast cancer that occurs. Breast cancer is the second most common cancer in the world with approximately 1.7 million new cases in 2012 and the fifth most common cause of death from cancer, with approximately 521,000 deaths. Of these cases, approximately 15% are triple-negative, which do not express the estrogen receptor, progesterone receptor (PR) or HER2. In some embodiments, triple negative breast cancer (TNBC) is characterized as breast cancer cells that are estrogen receptor expression negative (<1% of cells), progesterone receptor expression negative (<1% of cells), and HER2-negative. In one embodiment, the cancer is TNBC in PD-L1 positive patients having PD-L1 expressing tumor-infiltrating immune cells (IC) of ≥1%. The IC is as determined by an FDA- or EMA-approved test, such as the Ventana PD-L1 (SP142) assay.

In some embodiments, the cancer is estrogen receptor (ER)-positive breast cancer, ER-negative breast cancer, PR-positive breast cancer, PR-negative breast cancer, HER2-positive breast cancer, HER2-negative breast cancer, BRCA1/2-positive breast cancer, BRCA1/2-negative cancer, or TNBC. In some embodiments, the breast cancer is a metastatic breast cancer. In some embodiments, the breast cancer is an advanced breast cancer. In some embodiments, the cancer is a stage II, stage Ill or stage IV breast cancer. In some embodiments, the cancer is a stage IV breast cancer. In some embodiments, the breast cancer is a triple negative breast cancer.

In one embodiment, the cancer is endometrial cancer. Endometrial carcinoma is the most common cancer of the female genital, tract accounting for 10-20 per 100,000 person-years. The annual number of new cases of endometrial cancer (EC) is estimated at about 325 thousand worldwide. Further, EC is the most commonly occurring cancer in postmenopausal women. About 53% of endometrial cancer cases occur in developed countries. In 2015, approximately 55,000 cases of EC were diagnosed in the U.S. and no targeted therapies are currently approved for use in EC. There is a need for agents and regimens that improve survival for advanced and recurrent EC in 1L and 2L settings. Approximately 10,170 people are predicted to die from EC in the U.S. in 2016. The most common histologic form is endometrioid adenocarcinoma, representing about 75-80% of diagnosed cases. Other histologic forms include uterine papillary serous (less than 10%), clear cell 4%, mucinous 1%, squamous less than 1% and mixed about 10%.

From the pathogenetic point of view, EC falls into two different types, so-called types I and II. Type I tumors are low-grade and estrogen-related endometrioid carcinomas (EEC) while type II are non-endometrioid (NEEC) (mainly serous and clear cell) carcinomas. The World Health Organization has updated the pathologic classification of EC, recognizing nine different subtypes of EC, but EEC and serous carcinoma (SC) account for the vast majority of cases. EECs are estrogen-related carcinomas, which occur in perimenopausal patients, and are preceded by precursor lesions (endometrial hyperplasia/endometrioid intraepithelial neoplasia). Microscopically, lowgrade EEC (EEC 1-2) contains tubular glands, somewhat resembling the proliferative endometrium, with architectural complexity with fusion of the glands and cribriform pattern. High-grade EEC shows solid pattern of growth. In contrast, SC occurs in postmenopausal patients in absence of hyperestrogenism. At the microscope, SC shows thick, fibrotic or edematous papillae with prominent stratification of tumor cells, cellular budding, and anaplastic cells with large, eosinophilic cytoplasms. The vast majority of EEC are low grade tumors (grades 1 and 2), and are associated with good prognosis when they are restricted to the uterus. Grade 3 EEC (EEC3) is an aggressive tumor, with increased frequency of lymph node metastasis. SCs are very aggressive, unrelated to estrogen stimulation, mainly occurring in older women. EEC 3 and SC are considered high-grade tumors. SC and EEC3 have been compared using the surveillance, epidemiology and End Results (SEER) program data from 1988 to 2001. They represented 10% and 15% of EC respectively, but accounted for 39% and 27% of cancer death respectively. Endometrial cancers can also be classified into four molecular subgroups: (1) ultramutated/POLE-mutant; (2) hypermutated MSI+(e.g., MSI-H or MSI-L); (3) copy number low/micro satellite stable (MSS); and (4) copy number high/serous-like. Approximately 28% of cases are MSI-high. (Murali, Lancet Oncol. (2014). In some embodiments, the patient has a mismatch repair deficient subset of 2L endometrial cancer. In some embodiments, the endometrial cancer is metastatic endometrial cancer. In some embodiments, the patient has a MSS endometrial cancer. In some embodiments, the patient has a MSI-H endometrial cancer.

In one embodiment, the cancer is cervical cancer. In some embodiments, the cervical cancer is an advanced cervical cancer. In some embodiments, the cervical cancer is a metastatic cervical cancer. In some embodiments, the cervical cancer is a MSI-H cervical cancer. In some embodiments, the cervical cancer is a MSS cervical cancer. In some embodiments, the cervical cancer is a POLE-mutant cervical cancer. In some embodiments, the cervical cancer is a POLD-mutant cervical cancer. In some embodiments, the cervical cancer is a high TMB cervical cancer. In one embodiment, the cancer is cervical cancer in PD-L1 positive patients having a CPS ≥1%. The CPS is as determined by an FDA- or EMA-approved test, such as the Dako IHC 22C3 PharmDx assay.

In one embodiment, the cancer is uterine cancer. In some embodiments, the uterine cancer is an advanced uterine cancer. In some embodiments, the uterine cancer is a metastatic uterine cancer. In some embodiments, the uterine cancer is a MSI-H uterine cancer. In some embodiments, the uterine cancer is a MSS uterine cancer. In some embodiments, the uterine cancer is a POLE-mutant uterine cancer. In some embodiments, the uterine cancer is a POLD-mutant uterine cancer. In some embodiments, the uterine cancer is a high TMB uterine cancer.

In one embodiment, the cancer is urothelial cancer. In some embodiments, the urothelial cancer is an advanced urothelial cancer. In some embodiments, the urothelial cancer is a metastatic urothelial cancer. In some embodiments, the urothelial cancer is a MSI-H urothelial cancer. In some embodiments, the urothelial cancer is a MSS urothelial cancer. In some embodiments, the urothelial cancer is a POLE-mutant urothelial cancer. In some embodiments, the urothelial cancer is a POLD-mutant urothelial cancer. In some embodiments, the urothelial cancer is a high TMB urothelial cancer. In one embodiment, the cancer is urothelial carcinoma in PD-L1 positive patients having a CPS ≥10%. The CPS is as determined by an FDA- or EMA-approved test, such as the Dako IHC 22C3 PharmDx assay. In one embodiment, the cancer is urothelial carcinoma in PD-L1 positive patients having PD-L1 expressing tumor-infiltrating immune cells (IC) of ≥5%. The IC is as determined by an FDA- or EMA-approved test, such as the Ventana PD-L1 (SP142) assay.

In one embodiment, the cancer is thyroid cancer. In some embodiments, the thyroid cancer is an advanced thyroid cancer. In some embodiments, the thyroid cancer is a metastatic thyroid cancer. In some embodiments, the thyroid cancer is a MSI-H thyroid cancer. In some embodiments, the thyroid cancer is a MSS thyroid cancer. In some embodiments, the thyroid cancer is a POLE-mutant thyroid cancer. In some embodiments, the thyroid cancer is a POLD-mutant thyroid cancer. In some embodiments, the thyroid cancer is a high TMB thyroid cancer.

Tumors may be a hematopoietic (or hematologic or hematological or blood-related) cancer, for example, cancers derived from blood cells or immune cells, which may be referred to as “liquid tumors”. Specific examples of clinical conditions based on hematologic tumors include leukemias such as chronic myelocytic leukemia, acute myelocytic leukemia, chronic lymphocytic leukemia and acute lymphocytic leukemia; plasma cell malignancies such as multiple myeloma, monoclonal gammopathy of undetermined (or unknown or unclear) significance (MGUS) and Waldenstrom's macroglobulinemia; lymphomas such as non-Hodgkin's lymphoma, Hodgkin's lymphoma, and the like.

In one embodiment, the cancer is a gastric cancer (GC) or a gastroesophageal junction cancer (GEJ). In one embodiment, the cancer is GC or GEJ in PD-L1 positive patients having a CPS ≥1%. The CPS is as determined by an FDA- or EMA-approved test, such as the Dako IHC 22C3 PharmDx assay.

In one embodiment, the cancer is esophageal squamous cell carcinoma (ESCC). In one embodiment, the cancer is ESCC in PD-L1 positive patients having a CPS ≥10%. The CPS is as determined by an FDA- or EMA-approved test, such as the Dako IHC 22C3 PharmDx assay.

The cancer may be any cancer in which an abnormal number of blast cells or unwanted cell proliferation is present or that is diagnosed as a hematological cancer, including both lymphoid and myeloid malignancies. Myeloid malignancies include, but are not limited to, acute myeloid (or myelocytic or myelogenous or myeloblastic) leukemia (undifferentiated or differentiated), acute promyeloid (or promyelocytic or promyelogenous or promyeloblastic) leukemia, acute myelomonocytic (or myelomonoblastic) leukemia, acute monocytic (or monoblastic) leukemia, erythroleukemia and megakaryocytic (or megakaryoblastic) leukemia. These leukemias may be referred together as acute myeloid (or myelocytic or myelogenous) leukemia. Myeloid malignancies also include myeloproliferative disorders (MPD) which include, but are not limited to, chronic myelogenous (or myeloid or myelocytic) leukemia (CML), chronic myelomonocytic leukemia (CMML), essential thrombocythemia (or thrombocytosis), and polcythemia vera (PCV). Myeloid malignancies also include myelodysplasia (or myelodysplastic syndrome or MDS), which may be referred to as refractory anemia (RA), refractory anemia with excess blasts (RAEB), and refractory anemia with excess blasts in transformation (RAEBT); as well as myelofibrosis (MFS) with or without agnogenic myeloid metaplasia.

In one embodiment, the cancer is non-Hodgkin's lymphoma. Hematopoietic cancers also include lymphoid malignancies, which may affect the lymph nodes, spleens, bone marrow, peripheral blood, and/or extranodal sites. Lymphoid cancers include B-cell malignancies, which include, but are not limited to, B-cell non-Hodgkin's lymphomas (B-NHLs). B-NHLs may be indolent (or low-grade), intermediate-grade (or aggressive) or high-grade (very aggressive). Indolent B cell lymphomas include follicular lymphoma (FL); small lymphocytic lymphoma (SLL); marginal zone lymphoma (MZL) including nodal MZL, extranodal MZL, splenic MZL and splenic MZL with villous lymphocytes; lymphoplasmacytic lymphoma (LPL); and mucosa-associated-lymphoid tissue (MALT or extranodal marginal zone) lymphoma. Intermediate-grade B-NHLs include mantle cell lymphoma (MCL) with or without leukemic involvement, diffuse large B cell lymphoma (DLBCL), follicular large cell (or grade 3 or grade 3B) lymphoma, and primary mediastinal lymphoma (PML). High-grade B-NHLs include Burkitt's lymphoma (BL), Burkitt-like lymphoma, small non-cleaved cell lymphoma (SNCCL) and lymphoblastic lymphoma. Other B-NHLs include immunoblastic lymphoma (or immunocytoma), primary effusion lymphoma, HIV associated (or AIDS related) lymphomas, and post-transplant lymphoproliferative disorder (PTLD) or lymphoma. B-cell malignancies also include, but are not limited to, chronic lymphocytic leukemia (CLL), prolymphocytic leukemia (PLL), Waldenstrom's macroglobulinemia (WM), hairy cell leukemia (HCL), large granular lymphocyte (LGL) leukemia, acute lymphoid (or lymphocytic or lymphoblastic) leukemia, and Castleman's disease. NHL may also include T-cell non-Hodgkin's lymphomas (T-NHLs), which include, but are not limited to T-cell non-Hodgkin's lymphoma not otherwise specified (NOS), peripheral T-cell lymphoma (PTCL), anaplastic large cell lymphoma (ALCL), angioimmunoblastic lymphoid disorder (AILD), nasal natural killer (NK) cell/T-cell lymphoma, gamma/delta lymphoma, cutaneous T cell lymphoma, mycosis fungoides, and Sezary syndrome.

Hematopoietic cancers also include Hodgkin's lymphoma (or disease) including classical Hodgkin's lymphoma, nodular sclerosing Hodgkin's lymphoma, mixed cellularity Hodgkin's lymphoma, lymphocyte predominant (LP) Hodgkin's lymphoma, nodular LP Hodgkin's lymphoma, and lymphocyte depleted Hodgkin's lymphoma. Hematopoietic cancers also include plasma cell diseases or cancers such as multiple myeloma (MM) including smoldering MM, monoclonal gammopathy of undetermined (or unknown or unclear) significance (MGUS), plasmacytoma (bone, extramedullary), lymphoplasmacytic lymphoma (LPL), Waldenström's Macroglobulinemia, plasma cell leukemia, and primary amyloidosis (AL). Hematopoietic cancers may also include other cancers of additional hematopoietic cells, including polymorphonuclear leukocytes (or neutrophils), basophils, eosinophils, dendritic cells, platelets, erythrocytes and natural killer cells. Tissues which include hematopoietic cells referred herein to as “hematopoietic cell tissues” include bone marrow; peripheral blood; thymus; and peripheral lymphoid tissues, such as spleen, lymph nodes, lymphoid tissues associated with mucosa (such as the gut-associated lymphoid tissues), tonsils, Peyer's patches and appendix, and lymphoid tissues associated with other mucosa, for example, the bronchial linings.

In one embodiment, the treatment is first line or second line treatment of HNSCC. In one embodiment, the treatment is first line or second line treatment of recurrent/metastatic HNSCC. In one embodiment the treatment is first line treatment of recurrent/metastatic (1L R/M) HNSCC. In one embodiment, the treatment is first line treatment of 1L R/M HNSCC that is PD-L1 positive. In one embodiment the treatment is second line treatment of recurrent/metastatic (2L R/M) HNSCC.

In one embodiment, the treatment is first line, second line, third line, fourth line or fifth line treatment of PD-1/PD-L1-naïve HNSCC. In one embodiment, the treatment first line, second line, third line, fourth line or fifth line treatment of PD-1/PD-L1 experienced HNSCC.

In some embodiments, the treatment of cancer is first line treatment of cancer. In one embodiment, the treatment of cancer is second line treatment of cancer. In some embodiments, the treatment is third line treatment of cancer. In some embodiments, the treatment is fourth line treatment of cancer. In some embodiments, the treatment is fifth line treatment of cancer. In some embodiments, prior treatment to said second line, third line, fourth line or fifth line treatment of cancer comprises one or more of radiotherapy, chemotherapy, surgery or radiochemotherapy.

In one embodiment, the prior treatment comprises treatment with diterpenoids, such as paclitaxel, nab-paclitaxel or docetaxel; vinca alkaloids, such as vinblastine, vincristine, or vinorelbine; platinum coordination complexes, such as cisplatin or carboplatin; nitrogen mustards such as cyclophosphamide, melphalan, or chlorambucil; alkyl sulfonates such as busulfan; nitrosoureas such as carmustine; triazenes such as dacarbazine; actinomycins such as dactinomycin; anthrocyclins such as daunorubicin or doxorubicin; bleomycins; epipodophyllotoxins such as etoposide or teniposide; antimetabolite anti-neoplastic agents such as fluorouracil, methotrexate, cytarabine, mecaptopurine, thioguanine, or gemcitabine; methotrexate; camptothecins such as irinotecan or topotecan; rituximab; ofatumumab; trastuzumab; cetuximab; bexarotene; sorafenib; erbB inhibitors such as lapatinib, erlotinib or gefitinib; pertuzumab; ipilimumab; nivolumab; FOLFOX; capecitabine; FOLFIRI; bevacizumab; atezolizumab; selicrelumab; obinotuzumab or any combinations thereof. In one embodiment, prior treatment to said second line treatment, third line, fourth line or fifth line treatment of cancer comprises ipilimumab and nivolumab. In one embodiment, prior treatment to said second line treatment, third line, fourth line or fifth line treatment of cancer comprises FOLFOX, capecitabine, FOLFIRI/bevacizumab and atezolizumab/selicrelumab. In one embodiment, prior treatment to said second line treatment, third line, fourth line or fifth line treatment of cancer comprises carboplatin/Nab-paclitaxel. In one embodiment, prior treatment to said second line treatment, third line, fourth line or fifth line treatment of cancer comprises nivolumab and electrochemotherapy. In one embodiment, prior treatment to said second line treatment, third line, fourth line or fifth line treatment of cancer comprises radiotherapy, cisplatin and carboplatin/paclitaxel.

In one embodiment, the treatment is first line or second line treatment of head and neck cancer (in particular head and neck squamous cell carcinoma and oropharyngeal cancer). In one embodiment, the treatment is first line or second line treatment of recurrent/metastatic HNSCC. In one embodiment the treatment is first line treatment of recurrent/metastatic (1L R/M) HNSCC. In one embodiment, the treatment is first line treatment of 1L R/M HNSCC that is PD-L1 positive. In one embodiment the treatment is second line treatment of recurrent/metastatic (2L R/M) HNSCC.

In one embodiment, the treatment is first line, second line, third line, fourth line or fifth line treatment of PD-1/PD-L1-naïve HNSCC. In one embodiment, the treatment is first line, second line, third line, fourth line or fifth line treatment of PD-1/PD-L1 experienced HNSCC.

In some embodiments, the treatment results in one or more of increased tumor infiltrating lymphocytes including cytotoxic T cells, helper T cell and NK cells, increased T cells, increased granzyme B+ cells, reduced proliferating tumor cells and increased activated T cells as compared to levels prior to treatment (e.g. baseline level). Activated T cells may be observed by greater OX40 and human leukocyte antigen DR expression. In some embodiments, treatment results in upregulation of PD-1 and/or PD-L1 as compared to levels prior to treatment (e.g. baseline level).

In one embodiment, the methods of the present invention further comprise administering at least one neo-plastic agent or cancer adjuvant to said human. The methods of the present invention may also be employed with other therapeutic methods of cancer treatment.

Typically, any anti-neoplastic agent or cancer adjuvant that has activity versus a tumor, such as a susceptible tumor being treated may be co-administered in the treatment of cancer in the present invention. Examples of such agents can be found in Cancer Principles and Practice of Oncology by V. T. Devita, T. S. Lawrence, and S. A. Rosenberg (editors), 10th edition (Dec. 5, 2014), Lippincott Williams & Wilkins Publishers.

In one embodiment, the human has previously been treated with one or more different cancer treatment modalities. In some embodiments, at least some of the patients in the cancer patient population have previously been treated with one or more therapies, such as surgery, radiotherapy, chemotherapy or immunotherapy. In some embodiments, at least some of the patients in the cancer patient population have previously been treated with chemotherapy (e.g. platinum-based chemotherapy). For example, a patient who has received two lines of cancer treatment can be identified as a 2L cancer patient (e.g. a 2L NSCLC patient). In some embodiments, a patient has received two lines or more lines of cancer treatment (e.g. a 2L+ cancer patient such as a 2L+ endometrial cancer patient). In some embodiments, a patient has not been previously treated with an antibody therapy, such as an anti-PD-1 therapy. In some embodiments, a patient previously received at least one line of cancer treatment (e.g. a patient previously received at least one line or at least two lines of cancer treatment). In some embodiments, a patient previously received at least one line of treatment for metastatic cancer (e.g. a patient previously received one or two lines of treatment for metastatic cancer). In some embodiments, a subject is resistant to treatment with a PD-1 inhibitor. In some embodiments, a subject is refractory to treatment with a PD-1 inhibitor. In some embodiments, a method described herein sensitizes the subject to treatment with a PD-1 inhibitor.

In certain embodiments, the cancer to be treated is PD-L1 positive. For example, in certain embodiments, the cancer to be treated exhibits PD-L1+ expression (e.g., high PD-L1 expression). Methods of detecting a biomarker, such as PD-L1 or CD73 for example, on a cancer or tumor, are routine in the art and are contemplated herein. Non-limiting examples include immunohistochemistry, immunofluorescence and fluorescence activated cell sorting (FACS). In some embodiments, subjects or patients with PD-L1 high cancer are treated by intravenously administering anti-PD(L)1:TGFβRII fusion protein at a dose of about 1200 mg Q2W. In some embodiments, subjects or patients with PD-L1 high cancer are treated by intravenously administering anti-PD(L)1:TGFβRII fusion protein at a dose of about 1800 mg Q3W. In some embodiments, subjects or patients with PD-L1 high cancer are treated by intravenously administering anti-PD(L)1:TGFβRII fusion protein at a dose of about 2100 mg Q3W. In some embodiments, subjects or patients with PD-L1 high cancer are treated by intravenously administering anti-PD(L)1:TGFβRII fusion protein at a dose of about 2400 mg Q3W. In some embodiments, subjects or patients with PD-L1 high cancer are treated by intravenously administering anti-PD(L)1:TGFβRII fusion protein at a dose of about 15 mg/kg Q3W.

In certain embodiments, the cancer to be treated is CD73 positive. For example, in certain embodiments, the cancer to be treated exhibits CD73+ expression (e.g., high CD73 expression).

In certain embodiments, the cancer to be treated has elevated levels of adenosine in the tumor microenvironment.

In certain embodiments, the dosing regimen comprises administering the anti-PD(L)1:TGFβRII fusion protein, such as one having the amino acid sequence of bintrafusp alfa, at a dose of about 0.01-3000 mg (e.g. a dose about 0.01 mg; a dose about 0.08 mg; a dose about 0.1 mg; a dose about 0.24 mg; a dose about 0.8 mg; a dose about 1 mg; a dose about 2.4 mg; a dose about 8 mg; a dose about 10 mg; a dose about 20 mg; a dose about 24 mg; a dose about 30 mg; a dose about 40 mg; a dose about 48 mg; a dose about 50 mg; a dose about 60 mg; a dose about 70 mg; a dose about 80 mg; a dose about 90 mg; a dose about 100 mg; a dose about 160 mg; a dose about 200 mg; a dose about 240 mg; a dose about 300 mg; a dose about 400 mg; a dose about 500 mg; a dose about 600 mg; a dose about 700 mg; a dose about 800 mg; a dose about 900 mg; a dose about 1000 mg; a dose about 1100 mg; a dose about 1200 mg; a dose about 1300 mg; a dose about 1400 mg; a dose about 1500 mg; a dose about 1600 mg; a dose about 1700 mg; a dose about 1800 mg; a dose about 1900 mg; a dose about 2000 mg; a dose about 2100 mg; a dose about 2200 mg; a dose about 2300 mg; a dose about 2400 mg; a dose about 2500 mg; a dose about 2600 mg; a dose about 2700 mg; a dose about 2800 mg; a dose about 2900 mg; or a dose about 3000 mg). In some embodiments, the dose is a dose of about 500 mg. In some embodiments, the dose is about 1200 mg. In some embodiments, the dose is about 2400 mg. In some embodiments, the dose of the anti-PD(L)1:TGFβRII fusion protein, such as one having the amino acid sequence of bintrafusp alfa, is about 0.001-100 mg/kg (e.g., a dose about 0.001 mg/kg; a dose about 0.003 mg/kg; a dose about 0.01 mg/kg; a dose about 0.03 mg/kg; a dose about 0.1 mg/kg; a dose about 0.3 mg/kg; a dose about 1 mg/kg; a dose about 2 mg/kg; a dose about 3 mg/kg; a dose about 10 mg/kg; a dose about 15 mg/kg; or a dose about 30 mg/kg).

All fixed doses disclosed herein are considered comparable to the body-weight dosing based on a reference body weight of 80 kg. Accordingly, when reference is made to a fixed dose of 2400 mg, a body-weight dose of 30 mg/kg is likewise disclosed therewith.

In some embodiments, the anti-PD(L)1:TGFβRII fusion protein light chain and heavy chain sequences correspond to SEQ ID NO: 15 and SEQ ID NO: 17 or SEQ ID NO: 15 and SEQ ID NO: 18 respectively and the dose of the anti-PD(L)1:TGFβRII fusion protein is 30 mg/kg.

In one embodiment, the anti-PD(L)1:TGFβRII fusion protein, such as one having the amino acid sequence of bintrafusp alfa, is administered once every 2-6 weeks (e.g. 2, 3 or 4 weeks, in particular 3 weeks). In one embodiment, the anti-PD(L)1:TGFβRII fusion protein, such as one having the amino acid sequence of bintrafusp alfa, is administered for once every two weeks (“Q2W”). In one embodiment, the anti-PD(L)1:TGFβRII fusion protein, such as one having the amino acid sequence of bintrafusp alfa, is administered for once every three weeks (“Q3W”). In one embodiment, the anti-PD(L)1:TGFβRII fusion protein, such as one having the amino acid sequence of bintrafusp alfa, is administered for once every 6 weeks (“Q6W”). In one embodiment, the anti-PD(L)1:TGFβRII fusion protein, such as one having the amino acid sequence of bintrafusp alfa, is administered for Q3W for 2-6 dosing cycles (e.g. the first 3, 4, or 5 dosing cycles, in particular, the first 4 dosing cycles).

In some embodiments, the anti-PD(L)1:TGFβRII fusion protein light chain and heavy chain sequences correspond to SEQ ID NO: 15 and SEQ ID NO: 17 or SEQ ID NO: 15 and SEQ ID NO: 18 respectively and the anti-PD(L)1:TGFβRII fusion protein is administered Q3W.

In certain embodiments, about 1200 mg of the anti-PD(L)1:TGFβRII fusion protein, such as one having the amino acid sequence of bintrafusp alfa, is administered to a subject Q2W. In certain embodiments, about 2400 mg of the anti-PD(L)1:TGFβRII fusion protein, such as one having the amino acid sequence of bintrafusp alfa, is administered to a subject Q3W.

In some embodiments, the anti-PD(L)1:TGFβRII fusion protein light chain and heavy chain sequences correspond to SEQ ID NO: 15 and SEQ ID NO: 17 or SEQ ID NO: 15 and SEQ ID NO: 18 respectively and the anti-PD(L)1:TGFβRII fusion protein is administered at a dose of 30 mg/kg Q3W.

In some embodiments, the dosing regimen comprises administering the adenosine inhibitor at a dose of about 0.01-5000 mg (e.g. a dose about 0.01 mg; a dose about 0.08 mg; a dose about 0.1 mg; a dose about 0.24 mg; a dose about 0.8 mg; a dose about 1 mg; a dose about 2.4 mg; a dose about 8 mg; a dose about 10 mg; a dose about 20 mg; a dose about 24 mg; a dose about 30 mg; a dose about 40 mg; a dose about 48 mg; a dose about 50 mg; a dose about 60 mg; a dose about 70 mg; a dose about 80 mg; a dose about 90 mg; a dose about 100 mg; a dose about 160 mg; a dose about 200 mg; a dose about 240 mg; a dose about 300 mg; a dose about 400 mg; a dose about 500 mg; a dose about 600 mg; a dose about 700 mg; a dose about 800 mg; a dose about 900 mg; a dose about 1000 mg; a dose about 1100 mg; a dose about 1200 mg; a dose about 1300 mg; a dose about 1400 mg; a dose about 1500 mg; a dose about 1600 mg; a dose about 1700 mg; a dose about 1800 mg; a dose about 1900 mg; a dose about 2000 mg; a dose about 2100 mg; a dose about 2200 mg; a dose about 2300 mg; a dose about 2400 mg; a dose about 2500 mg; a dose about 2600 mg; a dose about 2700 mg; a dose about 2800 mg; a dose about 2900 mg; a dose about 3000 mg; a dose about 3100 mg; a dose about 3200 mg; a dose about 3300 mg; a dose about 3400 mg; a dose about 3500 mg; a dose about 3600 mg; a dose about 3700 mg; a dose about 3800 mg; a dose about 3900 mg; a dose about 4000 mg; a dose about 4100 mg; a dose about 4200 mg; a dose about 4300 mg; a dose about 4400 mg; a dose about 4500 mg; a dose about 4600 mg; a dose about 4700 mg; a dose about 4800 mg; a dose about 4900 mg; or a dose about 5000 mg). In some embodiments, the dose of the adenosine inhibitor is about 0.001-250 mg/kg (e.g., a dose about 0.001 mg/kg; a dose about 0.003 mg/kg; a dose about 0.01 mg/kg; a dose about 0.03 mg/kg; a dose about 0.1 mg/kg; a dose about 0.3 mg/kg; a dose about 1 mg/kg; a dose about 2 mg/kg; a dose about 3 mg/kg; a dose about 10 mg/kg; a dose about 15 mg/kg; a dose about 30 mg/kg or a dose about 100 mg/kg). In one embodiment, such doses of the adenosine inhibitor are administered orally.

In one embodiment, the adenosine inhibitor is administered one, two, three or four times a day. In one embodiment, the adenosine inhibitor is administered once daily (“QD”), particularly continuously. In one embodiment, the adenosine inhibitor is administered twice daily (“BID”), particularly continuously. In one embodiment, the adenosine inhibitor is administered three times per day (“TID”), particularly continuously. In one embodiment, the adenosine inhibitor is administered four times per day (“QID”), particularly continuously.

In one embodiment, the adenosine inhibitor is administered once every 2-6 weeks (e.g. 2, 3 or 4 weeks, in particular 3 weeks). In one embodiment, the adenosine inhibitor is administered for once every two weeks (“Q2W”). In one embodiment, the adenosine inhibitor is administered for once every three weeks (“Q3W”). In one embodiment, the adenosine inhibitor is administered for once every 6 weeks (“Q6W”). In one embodiment, the adenosine inhibitor is administered for Q3W for 2-6 dosing cycles (e.g. the first 3, 4, or 5 dosing cycles, in particular, the first 4 dosing cycles).

In certain embodiments, about 50-150 mg of the adenosine receptor inhibitor are administered BID. In certain embodiments, about 50-150 mg of the adenosine A_{2A} and/or A_{2B} receptor inhibitor, such as (S)-7-Oxa-2-aza-spiro[4.5]decane-2-carboxylic acid [7-(3,6-dihydro-2H-pyran-4-yl)-4-methoxy-thiazolo[4,5-c]pyridin-2-yl]-amide or a pharmaceutically acceptable salt, derivative, solvate, prodrug and stereoisomer thereof, including mixtures thereof in all ratios, is administered BID together with about 1200 mg of the anti-PD(L)1:TGFβRII fusion protein, such as one having the amino acid sequence of bintrafusp alfa, Q2W or about 2400 mg of the anti-PD(L)1:TGFβRII fusion protein, such as one having the amino acid sequence of bintrafusp alfa, Q3W.

Concurrent treatment in addition to the treatment with the combination of the invention and considered necessary for the patient's well-being may be given at discretion of the treating physician. In some embodiments, the present invention provides methods of treating, stabilizing or decreasing the severity or progression of one or more diseases or disorders described herein comprising administering to a patient in need thereof a PD-1 inhibitor, a TGFβ inhibitor, and an adenosine inhibitor in combination with an additional therapy, such as chemotherapy, radiotherapy or chemoradiotherapy.

In one embodiment, diterpenoids, such as paclitaxel, nab-paclitaxel or docetaxel; vinca alkaloids, such as vinblastine, vincristine, or vinorelbine; platinum coordination complexes, such as cisplatin or carboplatin; nitrogen mustards such as cyclophosphamide, melphalan, or chlorambucil; alkyl sulfonates such as busulfan; nitrosoureas such as carmustine; triazenes such as dacarbazine; actinomycins such as dactinomycin; anthrocyclins such as daunorubicin or doxorubicin; bleomycins; epipodophyllotoxins such as etoposide or teniposide; antimetabolite anti-neoplastic agents such as fluorouracil, pemetrexed, methotrexate, cytarabine, mecaptopurine, thioguanine, or gemcitabine; methotrexate; camptothecins such as irinotecan or topotecan; rituximab; ofatumumab; trastuzumab; cetuximab; bexarotene; sorafenib; erbB inhibitors such as lapatinib, erlotinib or gefitinib; pertuzumab; ipilimumab; tremelimumab; nivolumab; pembrolizumab; FOLFOX; capecitabine; FOLFIRI; bevacizumab; atezolizumab; selicrelumab; obinotuzumab or any combinations thereof is/are further administered concurrently or sequentially with the PD-1 inhibitor, TGFβ inhibitor, and adenosine inhibitor.

In one embodiment, chemotherapy is further administered concurrently or sequentially with the PD-1 inhibitor, TGFβ inhibitor, and adenosine inhibitor. In one embodiment, chemotherapy is further administered concurrently or sequentially with the PD-1 inhibitor, TGFβ inhibitor, and adenosine inhibitor. In one embodiment, the chemotherapy is platinum-based chemotherapy. In one embodiment, the chemotherapy is platinum-based chemotherapy and fluorouracil. In one embodiment, the platinum-based chemotherapy is paclitaxel, nab-paclitaxel, docetaxel, cisplatin, carboplatin or any combination thereof. In one embodiment, the platinum-based chemotherapy is fluorouracil, cisplatin, carboplatin or any combination thereof. In one embodiment, chemotherapy is a platinum doublet of cisplatin or carboplatin with any one of pemetrexed, paclitaxel, gemcitabine, or fluorouracil. In one embodiment chemotherapy is further administered concurrently or sequentially with the PD-1 inhibitor, TGFβ inhibitor, and adenosine inhibitor to PD-1 inhibitor naïve patients.

In one embodiment, the PD-1 inhibitor, TGFβ inhibitor, and adenosine inhibitor are administered concurrently or sequentially to PD-L1 positive and/or CD73 positive patients.

In one embodiment, radiotherapy is further administered concurrently or sequentially with the PD-1 inhibitor, TGFβ inhibitor, and adenosine inhibitor. In some embodiments, the radiotherapy is selected from the group consisting of systemic radiation therapy, external beam radiation therapy, image-guided radiation therapy, tomotherapy, stereotactic radio surgery, stereotactic body radiation therapy, and proton therapy. In some embodiments, the radiotherapy comprises external-beam radiation therapy, internal radiation therapy (brachytherapy), or systemic radiation therapy. See, e.g., Amini et al., Radiat Oncol.

“Stereotactic body radiation therapy (SBRT) for lung cancer patients previously treated with conventional radiotherapy: a review” 9:210 (2014); Baker et al., Radiat Oncol. “A critical review of recent developments in radiotherapy for non-small cell lung cancer” 11(1):115 (2016); Ko et al., Clin Cancer Res “The Integration of Radiotherapy with Immunotherapy for the Treatment of Non-Small Cell Lung Cancer” (24) (23) 5792-5806; and, Yamoah et al., Int J Radiat Oncol Biol Phys “Radiotherapy Intensification for Solid Tumors: A Systematic Review of Randomized Trials” 93(4): 737-745 (2015).

In some embodiments, the radiotherapy comprises external-beam radiation therapy, and the external bean radiation therapy comprises intensity-modulated radiation therapy (IMRT), image-guided radiation therapy (IGRT), tomotherapy, stereotactic radiosurgery, stereotactic body radiation therapy, proton therapy, or other charged particle beams.

In some embodiments, the radiotherapy comprises stereotactic body radiation therapy.

The PD-1 inhibitor, TGFβ inhibitor, and adenosine inhibitor are administered using any amount and any route of administration effective for treating or decreasing the severity of a disorder provided above. The exact amount required may vary from subject to subject, depending on the species, age, and general condition of the subject, the severity of the infection, the particular agent, its mode of administration, and the like.

In some embodiments, the PD-1 inhibitor, TGFβ inhibitor, and adenosine inhibitor are administered simultaneously, separately or sequentially and in any order. The PD-1 inhibitor, TGFβ inhibitor, and adenosine inhibitor are administered to the patient in any order (i.e., simultaneously or sequentially) and the compounds may be in separate compositions, formulations or unit dosage forms, or together in a single composition, formulation or unit dosage form. In one embodiment, the PD-1 inhibitor, TGFβ inhibitor, and adenosine inhibitor are administered simultaneously or sequentially in any order, in jointly therapeutically effective amounts (for example in synergistically effective amounts), e.g. in daily or intermittently dosages corresponding to the amounts described herein. The individual combination partners of the PD-1 inhibitor, TGFβ inhibitor, and adenosine inhibitor may be administered separately at different times during the course of therapy or concurrently. Typically, in such combination therapies, individual compounds are formulated into separate pharmaceutical compositions or medicaments. When the compounds are separately formulated, the individual compounds can be administered simultaneously or sequentially, optionally via different routes. Optionally, the treatment regimens for each of the PD-1 inhibitor, TGFβ inhibitor, and adenosine inhibitor have different but overlapping delivery regimens, e.g., daily, twice daily, vs. a single administration, or weekly. In certain embodiments, the PD-1 inhibitor, TGFβ inhibitor and adenosine inhibitor are administered simultaneously in the same composition comprising the PD-1 inhibitor, TGFβ inhibitor and adenosine inhibitor. In certain embodiments, the PD-1 inhibitor, TGFβ inhibitor and adenosine inhibitor are administered simultaneously in separate compositions, i.e., wherein the PD-1 inhibitor, TGFβ inhibitor and adenosine inhibitor are administered simultaneously each in a separate unit dosage form. In some embodiments, the PD-1 inhibitor and TGFβ inhibitor are fused and administered in a separate unit dosage form from the adenosine inhibitor and the PD-1 inhibitor and TGFβ inhibitor are administered simultaneously or sequentially in any order with the adenosine inhibitor. It will be appreciated that the PD-1 inhibitor, TGFβ inhibitor, and adenosine inhibitor are administered on the same day or on different days and in any order as according to an appropriate dosing protocol. The instant invention is therefore to be understood as embracing all such regimens of simultaneous or alternating treatment and the term “administering” is to be interpreted accordingly. In one embodiment, the PD-1 inhibitor and the TGFβ inhibitor are administered Q2W or Q3W and the adenosine inhibitor is administered BID.

In some embodiments, the anti-PD(L)1:TGFβRII fusion protein and the adenosine A_{2A} and/or A_{2B} receptor inhibitor are administered simultaneously, separately or sequentially and in any order. The anti-PD(L)1:TGFβRII fusion protein and the adenosine A_{2A} and/or A_{2B} receptor inhibitor are administered to the patient in any order (i.e., simultaneously or sequentially) in separate compositions, formulations or unit dosage forms, or together in a single composition, formulation or unit dosage form. In one embodiment, the anti-PD(L)1:TGFβRII fusion protein and the adenosine A_{2A} and/or A_{2B} receptor inhibitor are administered simultaneously or sequentially in any order, in jointly therapeutically effective amounts (for example in synergistically effective amounts), e.g. in daily or intermittently dosages corresponding to the amounts described herein. The individual combination partners of the anti-PD(L)1:TGFβRII fusion protein and the adenosine A_{2A} and/or A_{2B} receptor inhibitor may be administered separately at different times during the course of therapy or concurrently in divided or single combination forms. Typically, in such combination therapies, the individual compounds are formulated into separate pharmaceutical compositions or medicaments. When separately formulated, the individual compounds can be administered simultaneously or sequentially, optionally via different routes. Optionally, the treatment regimens for each of the anti-PD(L)1:TGFβRII fusion protein and the adenosine A_{2A} and/or A_{2B} receptor inhibitor have different but overlapping delivery regimens, e.g., daily, twice daily, vs. a single administration, or weekly. The anti-PD(L)1:TGFβRII fusion protein may be delivered prior to, substantially simultaneously with, or after the adenosine A_{2A} and/or A_{2B} receptor inhibitor. In certain embodiments, the anti-PD(L)1:TGFβRII fusion protein is administered simultaneously in the same composition comprising the anti-PD(L)1:TGFβRII fusion protein and the adenosine A_{2A} and/or A_{2B} receptor inhibitor. In certain embodiments, the anti-PD(L)1:TGFβRII fusion protein and the adenosine A_{2A} and/or A_{2B} receptor inhibitor are administered simultaneously in separate compositions, i.e., wherein the anti-PD(L)1:TGFβRII fusion protein and the adenosine A_{2A} and/or A_{2B} receptor inhibitor are administered simultaneously each in a separate unit dosage form. It will be appreciated that the anti-PD(L)1:TGFβRII fusion protein and the adenosine A_{2A} and/or A_{2B} receptor inhibitor are administered on the same day or on different days and in any order as according to an appropriate dosing protocol. In one embodiment, the anti-PD(L)1:TGFβRII fusion protein is administered Q2W or Q3W, e.g., by intravenous infusion or injection, and the adenosine A_{2A} and/or A_{2B} receptor inhibitor is administered orally BID. In one embodiment, the anti-PD(L)1:TGFβRII fusion protein is administered 1200 mg Q2W or 2400 mg Q3W, e.g., by intravenous infusion or injection, and the adenosine A_{2A} and/or A_{2B} receptor inhibitor is administered orally BID at 25-300 mg or 50-150 mg per dose.

In some embodiments, one or more of the PD-1 inhibitor, TGFβ inhibitor and adenosine inhibitor are administered to a patient in need of treatment at a first dose at a first interval for a first period and at a second dose at a second interval for a second period. Such first and second period could be the lead phase and maintenance phase of treatment. There may be a rest period between the first and second periods in one or more of the PD-1 inhibitor, TGFβ inhibitor and adenosine inhibitor in the combination is/are not administered to the patient. In some embodiments, there is a rest period between the first period and second period. In some embodiments, the rest period is between 1 day and 30 days. In some embodiments, the rest period is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 days. In some embodiments, the rest period is 1 week, 2 weeks, 3 weeks, 4 weeks, 5 weeks, 6 weeks, 7 weeks, 9 weeks, 10 weeks, 11 weeks, 12 weeks, 13 weeks, 14 weeks or 15 weeks.

In some embodiments, the first dose and second dose are the same for each of the PD-1 inhibitor, TGFβ inhibitor and adenosine inhibitor. In some embodiments, the first dose and second dose are different for each of the PD-1 inhibitor, TGFβ inhibitor and adenosine inhibitor. In some embodiments, the first dose and second dose of each of the PD-1 inhibitor and TGFβ inhibitor are the same, whereas the first and the second dose of the adenosine inhibitor are different. In some embodiments, the first dose and second dose of the adenosine inhibitor are the same, whereas the first and the second dose of each of the PD-1 inhibitor and TGFβ inhibitor are different.

In some embodiments, the first dose and the second dose of the anti-PD(L)1:TGFβRII fusion protein, e.g., one having the amino acid sequence of bintrafusp alfa, are about 1200 mg. In some embodiments, the first dose and the second dose of the anti-PD(L)1:TGFβRII fusion protein, e.g., one having the amino acid sequence of bintrafusp alfa, are about 2400 mg. In some embodiments, the first dose of the anti-PD(L)1:TGFβRII fusion protein, e.g., one having the amino acid sequence of bintrafusp alfa, is about 1200 mg and the second dose is about 2400 mg. In some embodiments, the first dose of the anti-PD(L)1:TGFβRII fusion protein, e.g., one having the amino acid sequence of bintrafusp alfa, is about 2400 mg and the second dose is about 1200 mg.

In some embodiments, the first interval and second interval are the same. In some embodiments, the first interval and the second interval are Q2W. In some embodiments, the first interval and the second interval are Q3W. In some embodiments, the first interval and the second interval are Q6W. In some embodiments, the first interval and the second interval are different. In some embodiments, the first interval is Q2W and the second interval is Q3W. In some embodiments, the first interval is Q3W and the second interval is Q6W.

In some embodiments, the first interval and second interval of the PD-1 inhibitor and TGFβ inhibitor are the same. In some embodiments, the first interval and the second interval of the PD-1 inhibitor and TGFβ inhibitor are Q2W. In some embodiments, the first interval and the second interval of the PD-1 inhibitor and TGFβ inhibitor are Q3W. In some embodiments, the first interval and the second interval of the PD-1 inhibitor and TGFβ inhibitor are Q6W. In some embodiments, the first interval and the second interval of the PD-1 inhibitor and TGFβ inhibitor are different. In some embodiments, the first interval of the PD-1 inhibitor and TGFβ inhibitor is Q2W and the second interval is Q3W. In some embodiments, the first interval of the PD-1 inhibitor and TGFβ inhibitor is Q3W and the second interval is Q6W.

In some embodiments, the anti-PD(L)1:TGFβRII fusion protein, e.g., one having the amino acid sequence of bintrafusp alfa, is administered at the first dose of 1200 mg Q2W for the first period of 2-6 dosing cycles (e.g. the first 3, 4, or 5 dosing cycles, in particular, the first 4 dosing cycles), and at the second dose of 2400 mg Q3W until therapy is discontinued (e.g. due to disease progression, an adverse event, or as determined by a physician). In some embodiments, the anti-PD(L)1:TGFβRII fusion protein, e.g., one having the amino acid sequence of bintrafusp alfa, is administered at the first dose of 1200 mg Q2W for the first three dosing cycles, and at the second dose of 2400 mg Q3W or more until therapy is discontinued (e.g. due to disease progression, an adverse event, or as determined by a physician). In some embodiments, the anti-PD(L)1:TGFβRII fusion protein, e.g., one having the amino acid sequence of bintrafusp alfa, is administered at the first dose of 1200 mg Q2W for the first four dosing cycles, and at the second dose of 2400 mg Q3W or more until therapy is discontinued (e.g. due to disease progression, an adverse event, or as determined by a physician). In some embodiments, the anti-PD(L)1:TGFβRII fusion protein, e.g., one having the amino acid sequence of bintrafusp alfa, is administered at the first dose of 1200 mg Q2W for the first five dosing cycles, and at the second dose of 2400 mg Q3W or more until therapy is discontinued (e.g. due to disease progression, an adverse event, or as determined by a physician).

It will be understood that there can be a first treatment with one or two compounds of the adenosine inhibitor, PD-1 inhibitor and TGFβ inhibitor, followed by the treatment with all three compounds. Between first administration to the patient of an adenosine inhibitor, a PD-1 inhibitor, a TGFβ inhibitor or a fused PD-1 inhibitor and TGFβ inhibitor as a monotherapy and the administration of the PD-1 inhibitor, TGFβ inhibitor and adenosine inhibitor as a combination therapy as described herein, a period of no treatment or no administration may be performed, such as for a defined number of cycles. For example, after first administration with a monotherapy, the patient may be administered no treatment for 1 cycle or 2 cycles of 3 weeks, 6 weeks or 12 weeks before being administered a combination therapy as described herein. Thus, in one embodiment, the patient is first administered an adenosine inhibitor as a monotherapy as described herein, then administered no treatment for 1 cycle or 2 cycles of 3 weeks, 6 weeks or 12 weeks, before the patient is administered an adenosine inhibitor with a PD-1 inhibitor and a TGFβ inhibitor as a combination therapy as described herein. In one embodiment, the patient is first administered a PD-1 inhibitor and/or a TGFβ inhibitor as a monotherapy as described herein, then administered no treatment for 1 cycle or 2 cycles of 3 weeks, 6 weeks or 12 weeks, before the patient is administered a PD-1 inhibitor, a TGFβ inhibitor with an adenosine inhibitor as a combination therapy as described herein.

Compositions of the present invention are administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, intraperitoneally, subcutaneously or intravenously. In one embodiment, the compositions are administered by intravenous infusion or injection. In another embodiment, the compositions are administered by intramuscular or subcutaneous injection. In one embodiment, the anti-PD(L)1:TGFβRII fusion protein is administered by intravenous infusion or injection. In another embodiment, the anti-PD(L)1:TGFβRII fusion protein is administered by intramuscular or subcutaneous injection. In one embodiment, the adenosine inhibitor is administered orally. In one embodiment, the adenosine inhibitor is administered by intravenous infusion or injection. In one embodiment, the anti-PD(L)1:TGFβRII fusion protein is administered by intravenous infusion or injection and the adenosine inhibitor is administered by intravenous infusion or injection. In one embodiment, the anti-PD(L)1:TGFβRII fusion protein is administered by intravenous infusion or injection and the adenosine inhibitor is administered orally.

In some embodiments, the anti-PD(L)1:TGFβRII fusion protein, e.g., one having the amino acid sequence of bintrafusp alfa, is administered intravenously (e.g., as an intravenous infusion) or subcutaneously. In some embodiments, the anti-PD(L)1:TGFβRII fusion protein, e.g., one having the amino acid sequence of bintrafusp alfa, is administered as an intravenous infusion. In some embodiments, the anti-PD(L)1:TGFβRII fusion protein, e.g., one having the amino acid sequence of bintrafusp alfa, is administered intravenously at a dose of about 1200 mg or about 2400 mg. In some embodiments, the anti-PD(L)1:TGFβRII fusion protein, e.g., one having the amino acid sequence of bintrafusp alfa, is administered intravenously at a dose of about 1200 mg Q2W. In some embodiments, the anti-PD(L)1:TGFβRII fusion protein, e.g., one having the amino acid sequence of bintrafusp alfa, is administered intravenously at a dose of about 2400 mg Q3W. In some embodiments, the anti-PD(L)1:TGFβRII fusion protein, e.g., one having the amino acid sequence of bintrafusp alfa, is administered intravenously at a dose of about 15 mg/kg Q3W.

In some embodiments, the adenosine inhibitor is an adenosine A_{2A} and/or A_{2B} receptor inhibitor and administered orally at one of the doses described above. In some embodiments, the adenosine inhibitor is an adenosine A_{2A} and/or A_{2B} receptor inhibitor and administered intravenously at one of the doses described above. In some embodiments, the adenosine inhibitor is an adenosine A_{2A} and/or A_{2B} receptor inhibitor and administered orally at 25-300 mg per dose BID. In some embodiments, the adenosine inhibitor is an adenosine A_{2A} and/or A_{2B} receptor inhibitor and administered intravenously at one of the doses described above Q2W or Q3W.

In some embodiments, the patient is first administered the anti-PD(L)1:TGFβRII fusion protein, e.g., one having the amino acid sequence of bintrafusp alfa, at a dose of about 1200 mg as a monotherapy regimen and then the anti-PD(L)1:TGFβRII fusion protein at a dose of about 1200 mg, with the adenosine inhibitor as a combination therapy regimen. In some embodiments, the patient is first administered the anti-PD(L)1:TGFβRII fusion protein, e.g., one having the amino acid sequence of bintrafusp alfa, at a dose of about 2400 mg as a monotherapy regimen and then the anti-PD(L)1:TGFβRII fusion protein at a dose of about 2400 mg, with the adenosine inhibitor as a combination therapy regimen. In some embodiments, the patient is first administered the adenosine inhibitor as a monotherapy regimen and then the adenosine inhibitor with the anti-PD(L)1:TGFβRII fusion protein, e.g., one having the amino acid sequence of bintrafusp alfa, at a dose of about 1200 mg, as a combination therapy regimen. In some embodiments, the patient is first administered the adenosine inhibitor as a monotherapy regimen and then the adenosine inhibitor with the anti-PD(L)1:TGFβRII fusion protein, e.g., one having the amino acid sequence of bintrafusp alfa, at a dose of about 2400 mg, as a combination therapy regimen.

In some embodiments, the combination regimen comprises the steps of: (a) under the direction or control of a physician, the subject receiving the PD-1 inhibitor and the TGFβ inhibitor prior to first receipt of the adenosine inhibitor; and (b) under the direction or control of a physician, the subject receiving the adenosine inhibitor. In some embodiments, the combination regimen comprises the steps of: (a) under the direction or control of a physician, the subject receiving the adenosine inhibitor prior to first receipt of the PD-1 inhibitor and the TGFβ inhibitor; and (b) under the direction or control of a physician, the subject receiving the PD-1 inhibitor and TGFβ inhibitor. In some embodiments, the combination regimen comprises the steps of: (a) under the direction or control of a physician, the subject receiving the PD-1 inhibitor prior to first receipt of the TGFβ inhibitor and the adenosine inhibitor; and (b) under the direction or control of a physician, the subject receiving the TGFβ inhibitor and the adenosine inhibitor. In some embodiments, the combination regimen comprises the steps of: (a) under the direction or control of a physician, the subject receiving the TGFβ inhibitor and the adenosine inhibitor prior to first receipt of the PD-1 inhibitor; and (b) under the direction or control of a physician, the subject receiving the PD-1 inhibitor. In some embodiments, the combination regimen comprises the steps of: (a) under the direction or control of a physician, the subject receiving the TGFβ inhibitor prior to first receipt of the PD-1 inhibitor and the adenosine inhibitor; and (b) under the direction or control of a physician, the subject receiving the PD-1 inhibitor and the adenosine inhibitor. In some embodiments, the combination regimen comprises the steps of: (a) under the direction or control of a physician, the subject receiving the PD-1 inhibitor and the adenosine inhibitor prior to first receipt of the TGFβ inhibitor; and (b) under the direction or control of a physician, the subject receiving the TGFβ inhibitor.

In some embodiments, the combination regimen comprises the steps of: (a) under the direction or control of a physician, the subject receiving the anti-PD(L)1 antibody and the TGFβRII or anti-TGFβ antibody prior to first receipt of the adenosine A_{2A} and/or A_{2B} receptor inhibitor; and (b) under the direction or control of a physician, the subject receiving the adenosine A_{2A} and/or A_{2B} receptor inhibitor. In some embodiments, the combination regimen comprises the steps of: (a) under the direction or control of a physician, the subject receiving the adenosine A_{2A} and/or A_{2B} receptor inhibitor prior to first receipt of the anti-PD(L)1 antibody and the TGFβRII or anti-TGFβ antibody; and (b) under the direction or control of a physician, the subject receiving the anti-PD(L)1 antibody and the TGFβRII or anti-TGFβ antibody. In some embodiments, the combination regimen comprises the steps of: (a) under the direction or control of a physician, the subject receiving the anti-PD(L)1 antibody prior to first receipt of the TGFβRII or anti-TGFβ antibody and the adenosine A_{2A} and/or A_{2B} receptor inhibitor; and (b) under the direction or control of a physician, the subject receiving the TGFβRII or anti-TGFβ antibody and the adenosine A_{2A} and/or A_{2B} receptor inhibitor. In some embodiments, the combination regimen comprises the steps of: (a) under the direction or control of a physician, the subject receiving the TGFβRII or anti-TGFβ antibody and the adenosine A_{2A} and/or A_{2B} receptor inhibitor prior to first receipt of the anti-PD(L)1 antibody; and (b) under the direction or control of a physician, the subject receiving the anti-PD(L)1 antibody. In some embodiments, the combination regimen comprises the steps of: (a) under the direction or control of a physician, the subject receiving the TGFβRII or anti-TGFβ antibody prior to first receipt of the anti-PD(L)1 antibody and the adenosine A_{2A} and/or A_{2B} receptor inhibitor; and (b) under the direction or control of a physician, the subject receiving the anti-PD(L)1 antibody and the adenosine A_{2A} and/or A_{2B} receptor inhibitor. In some embodiments, the combination regimen comprises the steps of: (a) under the direction or control of a physician, the subject receiving the anti-PD(L)1 antibody and the adenosine A_{2A} and/or A_{2B} receptor inhibitor prior to first receipt of the TGFβRII or anti-TGFβ antibody; and (b) under the direction or control of a physician, the subject receiving the TGFβRII or anti-TGFβ antibody.

In some embodiments, the combination regimen comprises the steps of: (a) under the direction or control of a physician, the subject receiving an anti-PD(L)1:TGFβRII fusion protein, e.g., having the amino acid sequence of bintrafusp alfa, prior to first receipt of an adenosine A_{2A} and/or A_{2B} receptor inhibitor, e.g., one according to any one of embodiments E1-E13; and (b) under the direction or control of a physician, the subject receiving the adenosine A_{2A} and/or A_{2B} receptor inhibitor. In some embodiments, the combination regimen comprises the steps of: (a) under the direction or control of a physician, the subject receiving an adenosine A_{2A} and/or A_{2B} receptor inhibitor, e.g., one according to any one of embodiments E1-E13, prior to first receipt of an anti-PD(L)1:TGFβRII fusion protein, e.g., having the amino acid sequence of bintrafusp alfa, (b) under the direction or control of a physician, the subject receiving the anti-PD(L)1:TGFβRII fusion protein. In some embodiments, the combination regimen comprises the steps of: (a) under the direction or control of a physician, the subject receiving an anti-PD(L)1:TGFβRII fusion protein, e.g., having the amino acid sequence of bintrafusp alfa, prior to first receipt of an adenosine A_{2A} and/or A_{2B} receptor inhibitor, e.g., one according to any one of embodiments E1-E13; and (b) under the direction or control of a physician, the subject receiving the adenosine A_{2A} and/or A_{2B} receptor inhibitor. In some embodiments, the combination regimen comprises the steps of: (a) under the direction or control of a physician, the subject receiving an adenosine A_{2A} and/or A_{2B} receptor inhibitor, e.g., one according to any one of embodiments E1-E13, prior to first receipt of an anti-PD(L)1:TGFβRII fusion protein, e.g., having the amino acid sequence of bintrafusp alfa, (b) under the direction or control of a physician, the subject receiving the anti-PD(L)1:TGFβRII fusion protein.

Also provided is a combination comprising a PD-1 inhibitor, a TGFβ inhibitor and an adenosine inhibitor. Also provided is a combination comprising an anti-PD(L)1 antibody, a TGFβRII or anti-TGFβ antibody, and an adenosine A_{2A} and/or A_{2B} receptor inhibitor. Also provided is a combination comprising an adenosine A_{2A} and/or A_{2B} receptor inhibitor and a fused PD-1 inhibitor and TGFβ inhibitor. Also provided is a combination comprising an anti-PD(L)1:TGFβRII fusion protein and an adenosine A_{2A} and/or A_{2B} receptor inhibitor. In some embodiments, any of said combinations is for use as a medicament or for use in the treatment of cancer.

It shall be understood that, in the various embodiments described above, the PD-1 inhibitor and the TGFβ inhibitor can be fused, e.g., as an anti-PD-L1:TGFβRII fusion protein or an anti-PD-1:TGFβRII fusion protein.

### Pharmaceutical Formulations and Kits

The PD-1 inhibitor, TGFβ inhibitor, and adenosine inhibitor described herein may also be in the form of pharmaceutical formulations or kits.

In some embodiments, the present invention provides a pharmaceutically acceptable composition comprising a PD-1 inhibitor. In some embodiments, the present invention provides a pharmaceutically acceptable composition comprising a TGFβ inhibitor. In some embodiments, the present invention provides a pharmaceutically acceptable composition comprising a fused PD-1 inhibitor and TGFβ inhibitor. In some embodiments, the present invention provides a pharmaceutically acceptable composition comprising anti-PD(L)1:TGFβRII fusion protein. In some embodiments, the present invention provides a pharmaceutically acceptable composition comprising anti-PD(L)1:TGFβRII fusion protein having the amino acid sequence of bintrafusp alfa. In some embodiments, the present invention provides a pharmaceutically acceptable composition comprising an adenosine inhibitor. In some embodiments, the present invention provides a pharmaceutically acceptable composition comprising an adenosine A_{2A} and/or A_{2B} receptor inhibitor. In some embodiments, the present invention provides a pharmaceutically acceptable composition comprising an adenosine inhibitor according to any one of embodiments E1-E13. In some embodiments, the present invention provides a pharmaceutical composition comprising a PD-1 inhibitor and a TGFβ inhibitor. In some embodiments, the present invention provides a pharmaceutical composition comprising a TGFβ inhibitor and an adenosine inhibitor. In some embodiments, the present invention provides a pharmaceutical composition comprising a PD-1 inhibitor and an adenosine inhibitor. In some embodiments, the present invention provides a pharmaceutical composition comprising a PD-1 inhibitor, a TGFβ inhibitor and an adenosine inhibitor. In some embodiments, the present invention provides a pharmaceutical composition comprising an adenosine inhibitor and a fused PD-1 inhibitor and TGFβ inhibitor. In some embodiments, the present invention provides a pharmaceutical composition comprising an anti-PD(L)1:TGFβRII fusion protein and an adenosine A_{2A} and/or A_{2B} receptor inhibitor. In some embodiments, the present invention provides a pharmaceutical composition comprising an anti-PD(L)1:TGFβRII fusion protein having the amino acid sequence of bintrafusp alfa and an adenosine inhibitor according to any one of embodiments E1-E13. The pharmaceutically acceptable composition may comprise at least a further pharmaceutically acceptable excipient or adjuvant, such as a pharmaceutically acceptable carrier.

In some embodiments, a composition comprising the fused PD-1 inhibitor and TGFβ inhibitor, e.g., an anti-PD(L)1:TGFβRII fusion protein, is separate from a composition comprising an adenosine inhibitor. In some embodiments, the PD-1 inhibitor and TGFβ inhibitor are fused e.g., as an anti-PD(L)1:TGFβRII fusion protein, and present with an adenosine inhibitor in the same composition.

Examples of such pharmaceutically acceptable compositions are described further below and herein.

The compositions of the present invention may be in a variety of forms. These include, for example, liquid, semi-solid and solid dosage forms, such as liquid solutions (e.g., injectable and infusible solutions), dispersions or suspensions, tablets, pills, powders, liposomes, and suppositories.

Pharmaceutically acceptable carriers, adjuvants or vehicles that are used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Liquid dosage forms for oral administration include, but are not limited to, pharmaceutically acceptable emulsions, microemulsions, solutions, suspensions, syrups and elixirs. The liquid dosage forms may additionally contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions, may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S. P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of the compounds of the invention, it is often desirable to slow absorption from subcutaneous or intramuscular injection. This may be accomplished by the use of a liquid suspension of crystalline or amorphous material with poor water solubility. The rate of absorption then depends upon its rate of dissolution that, in turn, may depend upon crystal size and crystalline form. Alternatively, delayed absorption of parenterally administered PD-1 inhibitor, TGFβ inhibitor and/or adenosine inhibitor, is accomplished by dissolving or suspending the compound in an oil vehicle. Injectable depot forms are made by forming microencapsulated matrices of PD-1 inhibitor, TGFβ inhibitor and/or adenosine inhibitor in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration can be suppositories, which can be prepared by mixing the compounds of this invention with suitable non-irritating excipients or carriers such as cocoa butter, polyethylene glycol or a suppository wax, which are solid at ambient temperature but liquid at body temperature and therefore melt in the rectum or vaginal cavity and release the active compound.

Dosage forms for oral administration include capsules, tablets, pills, powders, and granules, aqueous suspensions or solutions. In solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol and silicic acid, b) binders such as, for example, carboxymethylcellulose, alginates, gelatin, polyvinylpyrrolidinone, sucrose and acacia, c) humectants such as glycerol, d) disintegrating agents such as agar-agar, calcium carbonate, potato or tapioca starch, alginic acid, certain silicates and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hardfilled gelatin capsules using such excipients as lactose or milk sugar as well as high molecular weight polyethylene glycols and the like. The solid dosage forms of tablets, dragées, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

The PD-1 inhibitor, TGFβ inhibitor and/or adenosine inhibitor can also be in microencapsulated form with one or more excipients as noted above. The solid dosage forms of tablets, dragées, capsules, pills, and granules can be prepared with coatings and shells such as enteric coatings, release controlling coatings and other coatings well known in the pharmaceutical formulating art. In such solid dosage forms, the PD-1 inhibitor, TGFβ inhibitor and/or adenosine inhibitor may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of the PD-1 inhibitor, TGFβ inhibitor and/or adenosine inhibitor include ointments, pastes, creams, lotions, gels, powders, solutions, sprays, inhalants or patches. The active component is admixed under sterile conditions with a pharmaceutically acceptable carrier and any needed preservatives or buffers as may be required. Exemplary carriers for topical administration of compounds of this are mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2 octyldodecanol, benzyl alcohol and water. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Pharmaceutically acceptable compositions of this invention are optionally administered by nasal aerosol or inhalation. Such compositions are prepared according to techniques well-known in the art of pharmaceutical formulation and are prepared as solutions in saline, employing benzyl alcohol or other suitable preservatives, absorption promoters to enhance bioavailability, fluorocarbons, and/or other conventional solubilizing or dispersing agents.

In a further aspect, the invention relates to a kit comprising a PD-1 inhibitor and a package insert comprising instructions for using the PD-1 inhibitor in combination with an adenosine inhibitor, and a TGFβ inhibitor to treat or delay progression of a cancer in a subject. Also provided is a kit comprising an adenosine inhibitor and a package insert comprising instructions for using the adenosine inhibitor in combination with a PD-1 inhibitor, and a TGFβ inhibitor to treat or delay progression of a cancer in a subject. Also provided is a kit comprising a TGFβ inhibitor and a package insert comprising instructions for using the TGFβ inhibitor in combination with a PD-1 inhibitor, and an adenosine inhibitor to treat or delay progression of a cancer in a subject. Also provided is a kit comprising an anti-PD-L1 antibody and a package insert comprising instructions for using the anti-PD-L1 antibody in combination with an adenosine A_{2A} and/or A_{2B} receptor inhibitor, and a TGFβRII or anti-TGFβ antibody to treat or delay progression of a cancer in a subject. Also provided is a kit comprising an adenosine A_{2A} and/or A_{2B} receptor inhibitor and a package insert comprising instructions for using the adenosine A_{2A} and/or A_{2B} receptor inhibitor in combination with an anti-PD-L1 antibody, and a TGFβRII or anti-TGFβ antibody to treat or delay progression of a cancer in a subject. Also provided is a kit comprising a TGFβRII or anti-TGFβ antibody and a package insert comprising instructions for using the TGFβRII or anti-TGFβ antibody in combination with an anti-PD-L1 antibody, and an adenosine A_{2A} and/or A_{2B} receptor inhibitor to treat or delay progression of a cancer in a subject. Also provided is a kit comprising a PD-1 inhibitor and a TGFβ inhibitor, and a package insert comprising instructions for using the PD-1 inhibitor and the TGFβ inhibitor in combination with an adenosine inhibitor to treat or delay progression of a cancer in a subject. Also provided is a kit comprising an anti-PD-L1 antibody and a TGFβRII or anti-TGFβ antibody, and a package insert comprising instructions for using the anti-PD-L1 antibody and the TGFβRII or anti-TGFβ antibody in combination with an adenosine A_{2A} and/or A_{2B} receptor inhibitor to treat or delay progression of a cancer in a subject. Also provided is a kit comprising an anti-PD(L)1:TGFβRII fusion protein, e.g., one having the amino acid sequence of bintrafusp alfa, and a package insert comprising instructions for using the anti-PD(L)1:TGFβRII fusion protein in combination with an adenosine inhibitor according to any one of the embodiments E1-E13 to treat or delay progression of a cancer in a subject. Also provided is a kit comprising a PD-1 inhibitor and an adenosine inhibitor, and a package insert comprising instructions for using the PD-1 inhibitor and the adenosine inhibitor in combination with a TGFβ inhibitor to treat or delay progression of a cancer in a subject. Also provided is a kit comprising a TGFβ inhibitor and an adenosine inhibitor, and a package insert comprising instructions for using the TGFβ inhibitor and the adenosine inhibitor in combination with a PD-1 inhibitor to treat or delay progression of a cancer in a subject. Also provided is a kit comprising an anti-PD-L1 antibody and an adenosine A_{2A} and/or A_{2B} receptor inhibitor, and a package insert comprising instructions for using the anti-PD-L1 antibody and the adenosine A_{2A} and/or A_{2B} receptor inhibitor in combination with a TGFβRII or anti-TGFβ antibody to treat or delay progression of a cancer in a subject. Also provided is a kit comprising a TGFβRII or anti-TGFβ antibody and an adenosine A_{2A} and/or A_{2B} receptor inhibitor, and a package insert comprising instructions for using the TGFβRII or anti-TGFβ antibody and the adenosine A_{2A} and/or A_{2B} receptor inhibitor in combination with an anti-PD-L1 antibody to treat or delay progression of a cancer in a subject. Also provided is a kit comprising a PD-1 inhibitor, a TGFβ inhibitor and an adenosine A_{2A} and/or A_{2B} receptor inhibitor, and a package insert comprising instructions for using the PD-1 inhibitor, TGFβ inhibitor and the adenosine A_{2A} and/or A_{2B} receptor inhibitor to treat or delay progression of a cancer in a subject. Also provided is a kit comprising an anti-PD-L1 antibody, a TGFβRII or anti-TGFβ antibody and an adenosine A_{2A} and/or A_{2B} receptor inhibitor, and a package insert comprising instructions for using the anti-PD-L1 antibody, TGFβRII or anti-TGFβ antibody and adenosine A_{2A} and/or A_{2B} receptor inhibitor to treat or delay progression of a cancer in a subject. Also provided is a kit comprising an anti-PD(L)1:TGFβRII fusion protein, e.g., one having the amino acid sequence of bintrafusp alfa, and an adenosine inhibitor, e.g., according to any one of the embodiments E1-E13 and a package insert comprising instructions for using the anti-PD(L)1:TGFβRII fusion protein and the adenosine inhibitor to treat or delay progression of a cancer in a subject. The kit can comprise a first container, a second container, a third container and a package insert, wherein the first container comprises at least one dose of the PD-1 inhibitor, the second container comprises at least one dose of the adenosine inhibitor, the third container comprises at least one dose of the TGFβ inhibitor and the package insert comprises instructions for treating a subject for cancer using the three compounds. In some embodiments, the kit comprises a first container, a second container and a package insert, wherein the first container comprises at least one dose of an anti-PD(L)1:TGFβRII fusion protein, e.g., one having the amino acid sequence of bintrafusp alfa, the second container comprises at least one dose of an adenosine inhibitor, e.g., according to any one of the embodiments E1-E13 and the package insert comprises instructions for treating a subject for cancer using the two compounds. The first, second and third containers may be comprised of the same or different shape (e.g., vials, syringes and bottles) and/or material (e.g., plastic or glass). The kit may further comprise other materials that may be useful in administering the medicaments, such as diluents, filters, IV bags and lines, needles and syringes. The instructions can state that the medicaments are intended for use in treating a subject having a cancer that tests positive for PD-L1 and/or CD73, e.g., by means of an immunohistochemical (IHC) assay, FACS or LC/MS/MS.

Further Diagnostic, Predictive, Prognostic and/or Therapeutic Methods

The disclosure further provides diagnostic, predictive, prognostic and/or therapeutic methods using the PD-1 inhibitor, TGFβ inhibitor, and adenosine inhibitor described herein. Such methods are based, at least in part, on determination of the identity of the expression level of a biomarker of interest. In particular, the amount of any one of human PD-L1, CD73 and/or adenosine in a cancer patient sample can be used as a biomarker to predict whether the patient is likely to respond favorably to cancer therapy utilizing the therapeutic combination of the invention.

Any suitable sample can be used for the method. Non-limiting examples of such include one or more of a serum sample, plasma sample, whole blood, pancreatic juice sample, tissue sample, tumor lysate or a tumor sample, which can be an isolated from a needle biopsy, core biopsy and needle aspirate. For example, tissue, plasma or serum samples are taken from the patient before treatment and optionally on treatment with the therapeutic combination of the invention. The expression levels obtained on treatment are compared with the values obtained before starting treatment of the patient. The information obtained may be prognostic in that it can indicate whether a patient has responded favorably or unfavorably to cancer therapy.

It is to be understood that information obtained using the diagnostic assays described herein may be used alone or in combination with other information, such as, but not limited to, expression levels of other genes, clinical chemical parameters, histopathological parameters, or age, gender and weight of the subject. When used alone, the information obtained using the diagnostic assays described herein is useful in determining or identifying the clinical outcome of a treatment, selecting a patient for a treatment, or treating a patient, etc. When used in combination with other information, on the other hand, the information obtained using the diagnostic assays described herein is useful in aiding in the determination or identification of clinical outcome of a treatment, aiding in the selection of a patient for a treatment, or aiding in the treatment of a patient, and the like. In a particular aspect, the expression level can be used in a diagnostic panel each of which contributes to the final diagnosis, prognosis, or treatment selected for a patient.

Any suitable method can be used to measure the biomarker protein, DNA, RNA, or other suitable read-outs for biomarker levels, respectively, examples of which are described herein and/or are well known to the skilled artisan.

In some embodiments, determining the biomarker level comprises determining the biomarker expression. In some embodiments, the biomarker level is determined by the biomarker protein concentration in a patient sample, e.g., with biomarker specific ligands, such as antibodies or specific binding partners. The binding event can, e.g., be detected by competitive or non-competitive methods, including the use of a labeled ligand or biomarker specific moieties, e.g., antibodies, or labeled competitive moieties, including a labeled biomarker standard, which compete with labeled proteins for the binding event. If the biomarker specific ligand is capable of forming a complex with the biomarker, the complex formation can indicate biomarker expression in the sample. In various embodiments, the biomarker protein level is determined by a method comprising quantitative western blot, multiple immunoassay formats, ELISA, immunohistochemistry, histochemistry, or use of FACS analysis of tumor lysates, immunofluorescence staining, a bead-based suspension immunoassay, Luminex technology, or a proximity ligation assay. In one embodiment, the biomarker expression is determined by immunohistochemistry using one or more primary antibodies that specifically bind the biomarker.

In another embodiment, the biomarker RNA level is determined by a method comprising microarray chips, RT-PCR, qRT-PCR, multiplex qPCR or in-situ hybridization. In one embodiment of the invention, a DNA or RNA array comprises an arrangement of poly-nucleotides presented by or hybridizing to the biomarker gene immobilized on a solid surface. For example, to the extent of determining the biomarker mRNA, the mRNA of the sample can be isolated, if necessary, after adequate sample preparation steps, e.g., tissue homogenization, and hybridized with marker specific probes, in particular on a microarray platform with or without amplification, or primers for PCR-based detection methods, e.g., PCR extension labeling with probes specific for a portion of the marker mRNA.

Several approaches have been described for quantifying PD-L1 protein expression in IHC assays of tumor tissue sections (Thompson et al. (2004) PNAS 101(49): 17174; Thompson et al. (2006) Cancer Res. 66: 3381; Gadiot et al. (2012) Cancer 117: 2192; Taube et al. (2012) Sci Transl Med 4, 127ra37; and Toplian et al. (2012) New Eng. J Med. 366 (26): 2443). One approach employs a simple binary end-point of positive or negative for PD-L1 expression, with a positive result defined in terms of the percentage of tumor cells that exhibit histologic evidence of cell-surface membrane staining.

The level of biomarker mRNA expression may be compared to the mRNA expression levels of one or more reference genes that are frequently used in quantitative RT-PCR, such as ubiquitin C. In some embodiments, a level of biomarker expression (protein and/or mRNA) by malignant cells and/or by infiltrating immune cells within a tumor is determined to be “overexpressed” or “elevated” based on comparison with the level of biomarker expression (protein and/or mRNA) by an appropriate control. For example, a control biomarker protein or mRNA expression level may be the level quantified in non-malignant cells of the same type or in a section from a matched normal tissue.

In one embodiment, the efficacy of the therapeutic combination of the invention is predicted by means of PD-L1 expression in tumor samples. In one embodiment, the efficacy of the therapeutic combination of the invention is predicted by means of CD73 expression in tumor samples. In one embodiment, the efficacy of the therapeutic combination of the invention is predicted by means of adenosine expression in tumor samples.

This disclosure also provides a kit for determining if the combination of the invention is suitable for therapeutic treatment of a cancer patient, comprising means for determining a protein level of one or more of PD-L1, CD73 and/or adenosine or the expression level of its or their RNA, in a sample isolated from the patient and instructions for use. In another aspect, the kit further comprises a PD-1 inhibitor, a TGFβ inhibitor, and an adenosine inhibitor for therapy. In one aspect of the invention, the determination of a high PD-L1 level indicates increased PFS or OS when the patient is treated with the therapeutic combination of the invention. In one aspect of the invention, the determination of a high CD73 level indicates increased PFS or OS when the patient is treated with the therapeutic combination of the invention. In one aspect of the invention, the determination of a high adenosine level indicates increased PFS or OS when the patient is treated with the therapeutic combination of the invention. In one embodiment of the kit, the means for determining the biomarker protein level are antibodies with specific binding to the biomarker.

In still another aspect, the invention provides a method for advertising a PD-1 inhibitor in combination with a TGFβ inhibitor and an adenosine inhibitor, comprising promoting, to a target audience, the use of the combination for treating a subject with a cancer, optionally, based on the expression of one or more of PD-L1, CD73 and adenosine in samples taken from the subject. In still another aspect, the invention provides a method for advertising an adenosine inhibitor in combination with a PD-1 inhibitor and a TGFβ inhibitor, wherein the PD-1 inhibitor and TGFβ inhibitor are can be fused, comprising promoting, to a target audience, the use of the combination for treating a subject with a cancer, optionally, based on the expression of one or more of PD-L1, CD73 and adenosine in samples taken from the subject. In still another aspect, the invention provides a method for advertising a TGFβ inhibitor in combination with a PD-1 inhibitor and an adenosine inhibitor, comprising promoting, to a target audience, the use of the combination for treating a subject with a cancer, optionally, based on the expression of one or more of PD-L1, CD73 and adenosine in samples taken from the subject. In still another aspect, the invention provides a method for advertising an anti-PD(L)1:TGFβRII fusion protein, e.g., one having the amino acid sequence of bintrafusp alfa, in combination with an adenosine inhibitor, comprising promoting, to a target audience, the use of the combination for treating a subject with a cancer, optionally, based on the expression of one or more of PD-L1, CD73 and adenosine in samples taken from the subject. In still another aspect, the invention provides a method for advertising a combination comprising a PD-1 inhibitor, a TGFβ inhibitor and an adenosine inhibitor, comprising promoting, to a target audience, the use of the combination for treating a subject with a cancer, optionally, based on the expression of one or more of PD-L1, CD73 and adenosine in samples taken from the subject. Promotion may be conducted by any means available. In some embodiments, the promotion is by a package insert accompanying a commercial formulation of the therapeutic combination of the invention. The promotion may also be by a package insert accompanying a commercial formulation of the PD-1 inhibitor, TGFβ inhibitor, adenosine inhibitor or another medicament (when treatment is a therapy with the therapeutic combination of the invention and a further medicament). In some embodiments, the promotion is by a package insert where the package insert provides instructions to receive therapy with the therapeutic combination of the invention after measuring one or more of PD-L1, CD73 and adenosine expression levels, and in some embodiments, in combination with another medicament. In some embodiments, the promotion is followed by the treatment of the patient with the therapeutic combination of the invention with or without another medicament. In some embodiments, the package insert indicates that the therapeutic combination of the invention is to be used to treat the patient if the patient's cancer sample is characterized by one or more of high PD-L1, CD73 and adenosine biomarker levels. In some embodiments, the package insert indicates that the therapeutic combination of the invention is not to be used to treat the patient if the patient's cancer sample expresses one or more of low PD-L1, CD73 and adenosine biomarker levels. In some embodiments, a high PD-L1, CD73 and/or adenosine biomarker level means a measured PD-L1 level that correlates with a likelihood of increased PFS and/or OS when the patient is treated with the therapeutic combination of the invention, and vice versa. In some embodiments, the PFS and/or OS is decreased relative to a patient who is not treated with the therapeutic combination of the invention. In some embodiments, the promotion is by a package insert where the package insert provides instructions to receive therapy with an anti-PD(L)1:TGFβRII fusion protein in combination with an adenosine inhibitor after first measuring one or more of PD-L1, CD73 and adenosine expression levels. In some embodiments, the promotion is followed by the treatment of the patient with an anti-PD(L)1:TGFβRII fusion protein in combination with an adenosine inhibitor with or without another medicament.

### Further Embodiments of the Present Disclosure

1. A PD-1 inhibitor, a TGFβ inhibitor and an adenosine inhibitor for use in a method of treating a cancer in a subject, wherein the method comprises administering the PD-1 inhibitor, the TGFβ inhibitor and the adenosine inhibitor to the subject.
2. A PD-1 inhibitor, a TGFβ inhibitor and an adenosine inhibitor for use in a method of treating a cancer in a subject,
   wherein the method comprises administering the PD-1 inhibitor, the TGFβ inhibitor and the adenosine inhibitor to the subject; and
   wherein the PD-1 inhibitor is an anti-PD(L)1 antibody, the TGFβ inhibitor is a TGFβRII or anti-TGFβ antibody and the adenosine inhibitor is an adenosine A_{2A} and/or A_{2B} receptor inhibitor.
3. A PD-1 inhibitor, a TGFβ inhibitor and an adenosine inhibitor for use in a method of treating a cancer in a subject,
   wherein the method comprises administering the PD-1 inhibitor, the TGFβ inhibitor and the adenosine inhibitor to the subject; and
   wherein the PD-1 inhibitor and the TGFβ inhibitor are fused as an anti-PD(L)1:TGFβRII fusion protein and the adenosine inhibitor an adenosine A_{2A} and A_{2B} receptor inhibitor.
4. A PD-1 inhibitor for use in a method of treating a cancer in a subject, wherein the method comprises administering the PD-1 inhibitor to the subject in combination with a TGFβ inhibitor and an adenosine inhibitor.
5. A TGFβ inhibitor for use in a method of treating a cancer in a subject, wherein the method comprises administering the TGFβ inhibitor to the subject in combination with a PD-1 inhibitor and an adenosine inhibitor.
6. An adenosine inhibitor for use in a method of treating a cancer in a subject, wherein the method comprises administering the adenosine inhibitor to the subject in combination with a PD-1 inhibitor and a TGFβ inhibitor.
7. A PD-1 inhibitor and a TGFβ inhibitor for use in a method of treating a cancer in a subject, wherein the method comprises administering the PD-1 inhibitor and the TGFβ inhibitor to the subject in combination with an adenosine inhibitor; and
   wherein the PD-1 inhibitor and the TGFβ inhibitor are fused.
8. A method of treating a cancer in a subject, wherein the method comprises administering a PD-1 inhibitor, a TGFβ inhibitor and an adenosine inhibitor to the subject.
9. A method of treating a cancer in a subject, wherein the method comprises administering a PD-1 inhibitor, a TGFβ inhibitor and an adenosine inhibitor to the subject; and
   wherein the PD-1 inhibitor is an anti-PD(L)1 antibody, the TGFβ inhibitor is a TGFβRII or anti-TGFβ antibody and the adenosine inhibitor is an adenosine A_{2A} and/or A_{2B} receptor inhibitor.
10. A method of treating a cancer in a subject, wherein the method comprises administering a PD-1 inhibitor, a TGFβ inhibitor and an adenosine inhibitor to the subject; and
   wherein the PD-1 inhibitor and the TGFβ inhibitor are fused as an anti-PD(L)1:TGFβRII fusion protein and the adenosine inhibitor is an adenosine A_{2A} and A_{2B} receptor inhibitor.
11. Use of a PD-1 inhibitor, a TGFβ inhibitor and an adenosine inhibitor for the manufacture of a medicament for a method of treating a cancer in a subject, wherein the method comprises administering the PD-1 inhibitor, the TGFβ inhibitor and the adenosine inhibitor to the subject.
12. Use of a PD-1 inhibitor, a TGFβ inhibitor and an adenosine inhibitor for the manufacture of a medicament for a method of treating a cancer in a subject,
   wherein the method comprises administering the PD-1 inhibitor, the TGFβ inhibitor and the adenosine inhibitor to the subject; and
   wherein the PD-1 inhibitor is an anti-PD(L)1 antibody, the TGFβ inhibitor is a TGFβRII or anti-TGFβ antibody and the adenosine inhibitor is an adenosine A_{2A} and/or A_{2B} receptor inhibitor.
13. Use of a PD-1 inhibitor, a TGFβ inhibitor and an adenosine inhibitor for the manufacture of a medicament for a method of treating a cancer in a subject,
   wherein the method comprises administering the PD-1 inhibitor, the TGFβ inhibitor and the adenosine inhibitor to the subject; and
   wherein the PD-1 inhibitor and the TGFβ inhibitor are fused as an anti-PD(L)1:TGFβRII fusion protein and the adenosine inhibitor is an adenosine A_{2A} and A_{2B} receptor inhibitor.
14. Use of a PD-1 inhibitor for the manufacture of a medicament for a method of treating a cancer in a subject, wherein the method comprises administering the PD-1 inhibitor to the subject in combination with a TGFβ inhibitor and an adenosine inhibitor.
15. Use of a TGFβ inhibitor for the manufacture of a medicament for a method of treating a cancer in a subject, wherein the method comprises administering the TGFβ inhibitor to the subject in combination with a PD-1 inhibitor and an adenosine inhibitor.
16. Use of an adenosine inhibitor for the manufacture of a medicament for a method of treating a cancer in a subject, wherein the method comprises administering the adenosine inhibitor to the subject in combination with a PD-1 inhibitor and a TGFβ inhibitor.
17. Use of a PD-1 inhibitor and a TGFβ inhibitor for the manufacture of a medicament for a method of treating a cancer in a subject, wherein the method comprises administering the PD-1 inhibitor and the TGFβ inhibitor to the subject in combination with an adenosine inhibitor; and
   wherein the PD-1 inhibitor and the TGFβ inhibitor are fused.
18. The compounds for use, method of treatment or use according to any one of items 1 to 17, wherein the PD-1 inhibitor is capable of inhibiting the interaction between PD-1 and PD-L1.
19. The compounds for use, method of treatment or use according to item 18, wherein the PD-1 inhibitor is an anti-PD(L)1 antibody.
20. The compounds for use, method of treatment or use according to item 19, wherein the PD-1 inhibitor is an anti-PD-L1 antibody.
21. The compounds for use, method of treatment or use according to item 20, wherein the anti-PD-L1 antibody comprises a heavy chain sequence, which comprises a CDRH1 having the sequence of SEQ ID NO: 1, a CDRH2 having the sequence of SEQ ID NO: 2 and a CDRH3 having the sequence of SEQ ID NO: 3, and a light chain sequence, which comprises a CDRL1 having the sequence of SEQ ID NO: 4, a CDRL2 having the sequence of SEQ ID NO: 5 and a CDRL3 having the sequence of SEQ ID NO: 6.
22. The compounds for use, method of treatment or use according to any one of items 1 to 21, wherein the TGFβ inhibitor is capable of inhibiting the interaction between a TGFβ and a TGFβ receptor.
23. The compounds for use, method of treatment or use according to any one of items 1 to 22, wherein the TGFβ inhibitor is a TGFβ receptor or a fragment thereof capable of binding TGFβ.
24. The compounds for use, method of treatment or use according to item 23, wherein the TGFβ receptor is TGFβ receptor II or a fragment thereof capable of binding TGFβ.
25. The compounds for use, method of treatment or use according to item 24, wherein the TGFβ receptor is an extracellular domain of TGFβ receptor II or a fragment thereof capable of binding TGFβ.
26. The compounds for use, method of treatment or use according to any one of items 1 to 25, wherein the TGFβ inhibitor has at least 80%, 90%, 95%, or 100% sequence identity to the amino acid sequence of any one of SEQ ID NO: 11, SEQ ID NO: 12 and SEQ ID NO: 13 and is capable of binding TGFβ.
27. The compounds for use, method of treatment or use according to any one of items 1 to 26, wherein the TGFβ inhibitor has at least 80%, 90%, or 95% sequence identity to the amino acid sequence of SEQ ID NO: 11 and is capable of binding TGFβ.
28. The compounds for use, method of treatment or use according to any one of items 1 to 25, wherein the TGFβ inhibitor comprises the sequence of any one of SEQ ID NO: 11, SEQ ID NO: 12 and SEQ ID NO: 13.
29. The compounds for use, method of treatment or use according to item 28, wherein the TGFβ inhibitor comprises the sequence of SEQ ID NO: 11.
30. The compounds for use, method of treatment or use according to any one of items 1 to 29, wherein the PD-1 inhibitor and the TGFβ inhibitor are fused.
31. The compounds for use, method of treatment or use according to any one of items 1 to 30, wherein the PD-1 inhibitor and the TGFβ inhibitor are fused in a molecule comprising (a) an antibody or a fragment thereof capable of binding PD-L1 or PD-1 and inhibiting the interaction between PD-1 and PD-L1 and (b) the extracellular domain of TGFβRII or a fragment thereof capable of binding TGFβ and inhibiting the interaction between TGFβ and a TGFβ receptor.
32. The compounds for use, method of treatment or use according to item 31, wherein the fusion molecule is one of the respective fusion molecules disclosed in WO 2015/118175 or WO 2018/205985.
33. The compounds for use, method of treatment or use according to item 31, wherein the extracellular domain of the TGFβRII or the fragment thereof is fused to each of the heavy chain sequences of the antibody or the fragment thereof.
34. The compounds for use, method of treatment or use according to item 33, wherein the fusion between the extracellular domains of TGFβRII or fragments thereof and the heavy chain sequences of the antibody or the fragment thereof occurs via a linker sequence.
35. The compounds for use, method of treatment or use according to item 34, wherein the amino acid sequence of the light chain sequences and the sequences comprising the heavy chain sequence and the extracellular domain of TGFβRII or the fragment thereof respectively correspond to the sequences selected from the group consisting of: (1) SEQ ID NO: 7 and SEQ ID NO: 8, (2) SEQ ID NO: 15 and SEQ ID NO: 17, and (3) SEQ ID NO: 15 and SEQ ID NO: 18.
36. The compounds for use, method of treatment or use according to any one of items 1 to 35, wherein the PD-1 inhibitor and the TGFβ inhibitor are fused and the fusion protein has at least 80%, 90%, 95% or 100% sequence identity to the amino acid sequence of bintrafusp alfa.
37. The compounds for use, method of treatment or use according to any one of items 1 to 35, wherein the PD-1 inhibitor and the TGFβ inhibitor are fused and the fusion protein is bintrafusp alfa.
38. The compounds for use, method of treatment or use according to any one of items 1 to 37, wherein the adenosine inhibitor is an adenosine A_{2A} and/or A_{2B} receptor inhibitor.
39. The compounds for use, method of treatment or use according to any one of items 1 to 37, wherein the adenosine inhibitor is an adenosine A_{2A} and A_{2B} receptor inhibitor.
40. The compounds for use, method of treatment or use according to any one of items 1 to 39, wherein the adenosine inhibitor is a compound of the formula I,

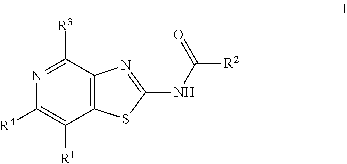

wherein
R¹ is linear or branched alkyl having 1-10 C atoms which is unsubstituted or mono-, di- or trisubstituted by R⁵ and in which 1-4 C atoms may be replaced, independently of one another, by O, S, SO, SO₂, NH, NCH₃, —OCO—, —NHCONH—, —NHCO—, —NR⁶SO₂R⁷—, —COO—, —CONH—, —NCH₃CO—, —CONCH₃—, —C≡C— groups and/or —CH═CH— groups, and/or, in addition, 1-10 H atoms may be replaced by F and/or Cl, or mono- or bicyclic cyclic alkyl having 3-7 C atoms which is unsubstituted or mono-, di- or trisubstituted by R⁵ and in which 1-4 C atoms may be replaced, independently of one another, by 0, S, SO, SO₂, NH, NCH₃, —OCO—, —NHCONH—, —NHCO—, —NR⁶SO₂R⁷—, —COO—, —CONH—, —NCH₃CO—, —CONCH₃—, —C≡C— groups and/or by —CH═CH— groups and/or, in addition, 1-10 H atoms may be replaced by F and/or Cl, or mono- or bicyclic heteroaryl, heterocyclyl, aryl or cyclic alkylaryl, containing 3 to 14 carbon atoms and 0-4 heteroatoms, independently selected from N, O and S, which is unsubstituted or mono-, di- or trisubstituted by R⁵,
R² is linear or branched alkyl having 1-10 C atoms which is unsubstituted or mono-, di- or trisubstituted by R⁵ and in which 1-4 C atoms may be replaced, independently of one another, by O, S, SO, SO₂, NH, NCH₃, —OCO—, —NHCONH—, —NHCO—, —NR⁶SO₂R⁷—, —COO—, —CONH—, —NCH₃CO—, —CONCH₃—, —C≡C— groups and/or —CH═CH— groups, and/or, in addition, 1-10 H atoms may be replaced by F and/or Cl, or cyclic alkyl having 3-7 C atoms which is unsubstituted or mono-, di- or trisubstituted by R⁵ and in which 1-4 C atoms may be replaced, independently of one another, by O, S, SO, SO₂, NH, NCH₃, —OCO—, —NHCONH—, —NHCO—, —NR⁶SO₂R⁷—, —COO—, —CONH—, —NCH₃CO—, —CONCH₃—, —C≡C— groups and/or by —CH═CH— groups and/or, in addition, 1-11 H atoms may be replaced by F and/or Cl, or mono- or bicyclic heteroaryl, heterocyclyl, aryl or cyclic alkylaryl, containing 3 to 14 carbon atoms and 0-4 heteroatoms, independently selected from N, O and S, which is unsubstituted or mono-, di- or trisubstituted by R⁵,
R³ is linear or branched alkyl or O-alkyl having 1-6 C atoms or cyclic alkyl having 3-6 C atoms, which is unsubstituted or mono-, di- or trisubstituted by H, ═S, ═NH, ═O, OH, cyclic alkyl having 3-6 C atoms, COOH, Hal, NH₂, SO₂CH₃, SO₂NH₂, CN, CONH₂, NHCOCH₃, NHCONH₂ or NO₂,
R⁴ is H, D, linear or branched alkyl having 1-6 C atoms or Hal,
R⁵ is H, R⁶, ═S, ═NR⁶, ═O, OH, COOH, Hal, NH₂, SO₂CH₃, SO₂NH₂, CN, CONH₂, NHCOCH₃, NHCONH₂, NO₂, or linear or branched alkyl having 1-10 C atoms which is unsubstituted or mono-, di- or trisubstituted by R⁶ and in which 1-4 C atoms may be replaced, independently of one another, by O, S, SO, SO₂, NH, NCH₃, —OCO—, —NHCONH—, —NHCO—, —NR⁶SO₂R⁷—, —COO—, —CONH—, —NCH₃CO—, —CONCH₃—, —C≡C— groups and/or —CH═CH— groups, and/or, in addition, 1-10 H atoms may be replaced by F and/or Cl, or mono- or bicyclic cyclic alkyl having 3-7 C atoms which is unsubstituted or mono-, di- or trisubstituted by R⁶ and in which 1-4 C atoms may be replaced, independently of one another, by O, S, SO, SO₂, NH, NCH₃, —OCO—, —NHCONH—, —NHCO—, —NR⁶SO₂R⁷—, —COO—, —CONH—, —NCH₃CO—, —CONCH₃—, —C≡C— groups and/or by —CH═CH— groups and/or, in addition, 1-10 H atoms may be replaced by F and/or Cl, or mono- or bicyclic heteroaryl, heterocyclyl, aryl or cyclic alkylaryl, containing 3 to 14 carbon atoms and 0-4 heteroatoms, independently selected from N, O and S, which is unsubstituted or mono-, di- or trisubstituted by R⁶,
   R⁶, R⁷ are independently of one another selected from the group consisting of H, ═S, ═NH, ═O, OH, COOH, Hal, NH₂, SO₂CH₃, SO₂NH₂, CN, CONH₂, NHCOCH₃, NHCONH₂, NO₂ and linear or branched alkyl having 1-10 C atoms in which 1-4 C atoms may be replaced, independently of one another, by O, S, SO, SO₂, NH, NCH₃, —OCO—, —NHCONH—, —NHCO—, —COO—, —CONH—, —NCH₃CO—, —CONCH₃—, —C≡C— groups and/or —CH═CH— groups, and/or, in addition, 1-10 H atoms may be replaced by F and/or Cl,
   Hal is F, Cl, Br, or I,
   D is deuterium
   and pharmaceutically acceptable salts, derivatives, solvates, prodrugs and stereoisomers thereof, including mixtures thereof in all ratios.
41. The compounds for use, method of treatment or use according to any one of items 1 to 40, wherein the adenosine inhibitor is an adenosine inhibitor according to any one of the embodiments E1-E13.
42. An adenosine inhibitor for use in a method of treating a cancer in a subject, wherein the method comprises administering the adenosine inhibitor to the subject in combination with a PD-1 inhibitor and a TGFβ inhibitor;
wherein the PD-1 and the TGFβ inhibitor are fused and the amino acid sequence of the fusion molecule corresponds to the amino acid sequence of bintrafusp alfa; and
   wherein the adenosine inhibitor is an adenosine inhibitor according to any one of the embodiments E1-E13.
43. A PD-1 inhibitor and a TGFβ inhibitor for use in a method of treating a cancer in a subject, wherein the method comprises administering the PD-1 inhibitor and the TGFβ inhibitor to the subject in combination with an adenosine inhibitor; and
   wherein the PD-1 and the TGFβ inhibitor are fused and the amino acid sequence of the fusion molecule corresponds to the amino acid sequence of bintrafusp alfa; and
   wherein the adenosine inhibitor is an adenosine inhibitor according to any one of the embodiments E1-E13.
44. A method of treating a cancer in a subject, wherein the method comprises administering a PD-1 inhibitor, a TGFβ inhibitor and an adenosine inhibitor to the subject;
   wherein the PD-1 and the TGFβ inhibitor are fused and the amino acid sequence of the fusion molecule corresponds to the amino acid sequence of bintrafusp alfa; and
   wherein the adenosine inhibitor is an adenosine inhibitor according to any one of the embodiments E1-E13.
45. Use of a PD-1 inhibitor, a TGFβ inhibitor and an adenosine inhibitor for the manufacture of a medicament for a method of treating a cancer in a subject, wherein the method comprises administering the PD-1 inhibitor, the TGFβ inhibitor and the adenosine inhibitor to the subject;
   wherein the PD-1 and the TGFβ inhibitor are fused and the amino acid sequence of the fusion molecule corresponds to the amino acid sequence of bintrafusp alfa; and
   wherein the adenosine inhibitor is an adenosine inhibitor according to any one of the embodiments E1-E13.
46. Use of an adenosine inhibitor for the manufacture of a medicament for a method of treating a cancer in a subject, wherein the method comprises administering the adenosine inhibitor to the subject in combination with a PD-1 inhibitor and a TGFβ inhibitor;
   wherein the PD-1 and the TGFβ inhibitor are fused and the amino acid sequence of the fusion molecule corresponds to the amino acid sequence of bintrafusp alfa; and
   wherein the adenosine inhibitor is an adenosine inhibitor according to any one of the embodiments E1-E13.
47. Use of a PD-1 inhibitor and a TGFβ inhibitor for the manufacture of a medicament for a method of treating a cancer in a subject, wherein the method comprises administering the PD-1 inhibitor and the TGFβ inhibitor to the subject in combination with an adenosine inhibitor;
   wherein the PD-1 and the TGFβ inhibitor are fused and the amino acid sequence of the fusion molecule corresponds to the amino acid sequence of bintrafusp alfa; and
   wherein the adenosine inhibitor is an adenosine inhibitor according to any one of the embodiments E1-E13.
48. The compounds for use, method of treatment or use according to any one of items 1 to 47, wherein the cancer is selected from the group consisting of carcinoma, lymphoma, leukemia, blastoma, and sarcoma.
49. The compounds for use, method of treatment or use according to any one of items 1 to 48, wherein the cancer is selected from the group consisting of squamous cell carcinoma, myeloma, small-cell lung cancer, non-small cell lung cancer, glioma, Hodgkin's lymphoma, non-Hodgkin's lymphoma, acute myeloid leukemia, multiple myeloma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma, cervical cancer, brain cancer, stomach cancer, bladder cancer, hepatoma, breast cancer, colon carcinoma, biliary tract cancer, and head and neck cancer.
50. The compounds for use, method of treatment or use according to any one of items 1 to 49, wherein the cancer has high adenosine-mediated signaling.
51. The compounds for use, method of treatment or use according to any one of items 1 to 50, wherein the cancer is adenosine-rich.
52. The compounds for use, method of treatment or use according to item 51, wherein the adenosine-rich cancer as has at least 0.5 μM, at least 0.75 μM, at least 1 μM, at least 1.5 μM, at least 2 μM, at least 5 μM or at least 10 μM adenosine in the tumor microenvironment.
53. The compounds for use, method of treatment or use according to any one of items 1 to 52, wherein the cancer has adenosine A_{2B} receptor-mediated signaling.
54. The compounds for use, method of treatment or use according to any one of items 1 to 53, wherein the cancer has adenosine-mediated signaling that exerts an immunosuppressive effect.
55. The compounds for use, method of treatment or use according to any one of items 1 to 54, wherein the cancer has an adenosine gene-expression signature.
56. The compounds for use, method of treatment or use according to item 55, wherein the adenosine gene-expression signature comprises evaluating the expression of CD73 and/or tissue non-specific alkaline phosphatase (TNAP).
57. The compounds for use, method of treatment or use according to item 55 or 56, wherein the adenosine gene-expression signature comprises evaluating the expression of one or more of CXCL1, CXCL2, CXCL3, CXCL5, CXCL6, CXCL8, IL1p and PTGS2.
58. The compounds for use, method of treatment or use according to any one of items 55 to 58, wherein the adenosine gene-expression signature is measured in peripheral blood or a cancer sample.
59. The compounds for use, method of treatment or use according to any one of items 55 to 58, wherein the adenosine gene-expression signature is measured in peripheral blood mononuclear cells.
60. The compounds for use, method of treatment or use according to any one of items 1 to 59, wherein the cancer is CD73 positive.
61. The compounds for use, method of treatment or use according to item 60, wherein at least 1%, at least 5%, at least 10%, at least 25%, at least 50% or at least 75% of the cells in the tumor microenvironment have CD73 present at their cell surface.
62. The compounds for use, method of treatment or use according to item 60, wherein the number of CD73 proteins per cell in a CD73 positive cancer is at least 1000, at least 5000, at least 10 000, at least 20 000 or at least 40 000.
63. The compounds for use, method of treatment or use according to item 60, wherein the CD73 expression is at least as high as the CD73 expression of one the cell lines selected from the group consisting of E0771 (ATCC CRL-3461), EMT6 (ATCC CRL-2755) and 4T1 (ATCC CRL-2539).
64. The compounds for use, method of treatment or use according to item 60, wherein the CD73 positive cancer is a cancer for which a separate peak is observed in a FACS plot when a sample of the cancer is analyzed using a fluorescently-labelled anti-CD73 antibody as compared to the respective isotype control.
65. The compounds for use, method of treatment or use according to any one of items 1 to 64, wherein the PD-1 inhibitor, TGFβ inhibitor and adenosine inhibitor are administered in a first line treatment of the cancer.
66. The compounds for use, method of treatment or use according to any one of items 1 to 64, wherein the subject underwent at least one round of prior cancer therapy.
67. The compounds for use, method of treatment or use according item 66, wherein the cancer was resistant or became resistant to prior therapy.
68. The compounds for use, method of treatment or use according to any one of items 1 to 64, wherein the PD-1 inhibitor, TGFβ inhibitor and adenosine inhibitor are administered in a second line or higher treatment of the cancer.
69. The compounds for use, method of treatment or use according to item 68, wherein the cancer is selected from the group consisting of pre-treated relapsing metastatic NSCLC, unresectable locally advanced NSCLC, pre-treated SCLC ED, SCLC unsuitable for systemic treatment, pre-treated relapsing or metastatic SCCHN, recurrent SCCHN eligible for re-irradiation, and pre-treated microsatellite status instable low (MSI-L) or microsatellite status stable (MSS) metastatic colorectal cancer (mCRC).
70. The compounds for use, method of treatment or use according to any one of items 1 to 69, wherein the PD-L1 inhibitor and the TGFβ inhibitor are fused and administered via intravenous infusion.
71. The compounds for use, method of treatment or use according to any one of items 1 to 70, wherein the PD-L1 inhibitor and the TGFβ inhibitor are fused and administered at a dose of about 1200 mg or about 2400 mg.
72. The compounds for use, method of treatment or use according to any one of items 1 to 71, wherein the PD-L1 inhibitor and the TGFβ inhibitor are fused and administered Q2W with a dose of about 1200 mg, or Q3W with a dose of about 2400 mg.
73. The compounds for use, method of treatment or use according to any one of items 1 to 72, wherein the adenosine inhibitor is administered orally.
74. The compounds for use, method of treatment or use according to any one of items 1 to 73, wherein the adenosine inhibitor is administered at a dose of about 25-300 mg or a dose of about 50-150 mg.
75. The compounds for use, method of treatment or use according to any one of items 1 to 74, wherein the adenosine inhibitor is administered BID.
76. The compounds for use, method of treatment or use according to any one of items 1 to 75, wherein the method comprises a lead phase, optionally followed by a maintenance phase after completion of the lead phase.
77. The compounds for use, method of treatment or use according to item 76, wherein the compounds are administered concurrently in either the lead or maintenance phase and optionally non-concurrently in the other phase, or the compounds are administered non-concurrently in the lead and maintenance phase, or two of the compounds are administered concurrently and the others non-concurrently in the lead and maintenance phase.
78. The compounds for use, method of treatment or use according to item 77, wherein the concurrent administration occurs sequentially in either order or substantially simultaneously.
79. The compounds for use, method of treatment or use according to any one of items 76 to 78, wherein the PD-1 inhibitor and TGFβ inhibitor are fused and the maintenance phase comprises administration of the fused PD-1 inhibitor and TGFβ inhibitor alone or concurrently with the adenosine inhibitor.
80. The compounds for use, method of treatment or use according to any one of items 76 to 79, wherein the lead phase comprises the concurrent administration of the PD-1 inhibitor, TGFβ inhibitor and adenosine inhibitor.
81. The compounds for use, method of treatment or use according to any one of items 1 to 80, wherein the cancer is selected based on PD-L1 expression in samples taken from the subject.
82. The compounds for use, method of treatment or use according to any one of items 1 to 81, wherein the cancer is selected based on CD73 expression in samples taken from the subject.
83. The compounds for use, method of treatment or use according to any one of items 1 to 82, wherein the cancer is selected based on the level of adenosine in samples taken from the subject.
84. A pharmaceutical composition comprising a PD-1 inhibitor, a TGFβ inhibitor and an adenosine inhibitor and at least a pharmaceutically acceptable excipient or adjuvant.
85. A pharmaceutical composition comprising a PD-1 inhibitor, a TGFβ inhibitor and an adenosine inhibitor and at least a pharmaceutically acceptable excipient or adjuvant;
   wherein the PD-1 inhibitor is an anti-PD(L)1 antibody, the TGFβ inhibitor is a TGFβRII or anti-TGFβ antibody and the adenosine inhibitor is an adenosine A_{2A} and/or A_{2B} receptor inhibitor.
86. A pharmaceutical composition comprising a PD-1 inhibitor, a TGFβ inhibitor and an adenosine inhibitor and at least a pharmaceutically acceptable excipient or adjuvant;
   wherein the PD-1 inhibitor and the TGFβ inhibitor are fused as an anti-PD(L)1:TGFβRII fusion protein and the adenosine inhibitor is an adenosine A_{2A} and A_{2B} receptor inhibitor.
87. A pharmaceutical composition comprising a PD-1 inhibitor, a TGFβ inhibitor and an adenosine inhibitor and at least a pharmaceutically acceptable excipient or adjuvant;
   wherein the PD-1 inhibitor and the TGFβ inhibitor are fused as an anti-PD(L)1:TGFβRII fusion protein having the amino acid sequence of bintrafusp alfa and the adenosine inhibitor is an adenosine inhibitor according to any one of the embodiments E1-E13.
88. The pharmaceutical composition according to any one of items 84 to 87 for use in therapy, e.g., for use in treating cancer.
89. A kit comprising a PD-1 inhibitor and a package insert comprising instructions for using the PD-1 inhibitor in combination with an adenosine inhibitor and a TGFβ inhibitor to treat or delay progression of a cancer in a subject.
90. A kit comprising an adenosine inhibitor and a package insert comprising instructions for using the adenosine inhibitor in combination with a PD-1 inhibitor and a TGFβ inhibitor to treat or delay progression of a cancer in a subject.
91. A kit comprising a TGFβ inhibitor and a package insert comprising instructions for using the TGFβ inhibitor in combination with a PD-1 inhibitor and an adenosine inhibitor to treat or delay progression of a cancer in a subject.
92. A kit comprising a PD-1 inhibitor and a package insert comprising instructions for using the PD-1 inhibitor in combination with an adenosine inhibitor and a TGFβ inhibitor to treat or delay progression of a cancer in a subject;
   wherein the PD-1 inhibitor is an anti-PD(L)1 antibody, the TGFβ inhibitor is a TGFβRII or anti-TGFβ antibody and the adenosine inhibitor is an adenosine A_{2A} and/or A_{2B} receptor inhibitor.
93. A kit comprising an adenosine inhibitor and a package insert comprising instructions for using the VEGF inhibitor in combination with a PD-1 inhibitor and a TGFβ inhibitor to treat or delay progression of a cancer in a subject;
   wherein the PD-1 inhibitor is an anti-PD(L)1 antibody, the TGFβ inhibitor is a TGFβRII or anti-TGFβ antibody and the adenosine inhibitor is an adenosine A_{2A} and/or A_{2B} receptor inhibitor.
94. A kit comprising a TGFβ inhibitor and a package insert comprising instructions for using the TGFβ inhibitor in combination with a PD-1 inhibitor and an adenosine inhibitor to treat or delay progression of a cancer in a subject;
   wherein the PD-1 inhibitor is an anti-PD(L)1 antibody, the TGFβ inhibitor is a TGFβRII or anti-TGFβ antibody and the adenosine inhibitor is an adenosine A_{2A} and/or A_{2B} receptor inhibitor.
95. A kit comprising a PD-1 inhibitor, a TGFβ inhibitor and a package insert comprising instructions for using the PD-1 inhibitor and the TGFβ inhibitor in combination with an adenosine inhibitor to treat or delay progression of a cancer in a subject;
   wherein the PD-1 inhibitor and the TGFβ inhibitor are fused as an anti-PD(L)1:TGFβRII fusion protein and the adenosine inhibitor is an adenosine A_{2A} and/or A_{2B} receptor inhibitor.
96. The kit according to any one of items 89 to 95, wherein the instructions state that the medicaments are intended for use in treating a subject having a cancer that tests positive for PD-L1 expression.
97. The kit according to any one of items 89 to 95, wherein the instructions state that the medicaments are intended for use in treating a subject having a cancer that tests positive for CD73 expression.
98. A method for advertising a PD-1 inhibitor, a TGFβ inhibitor and an adenosine inhibitor comprising promoting, to a target audience, the use of the combination for treating a subject with a cancer, such as a cancer selected based on PD-L1 or CD73 expression or the levels of adenosine in samples taken from the subject.

All the references cited herein are incorporated by reference in the disclosure of the invention hereby.

Although methods and materials similar or equivalent to those described herein can be used in the practice or testing of the present invention, suitable examples are described below. Within the examples, standard reagents and buffers that are free from contaminating activities (whenever practical) are used. The examples are particularly to be construed such that they are not limited to the explicitly demonstrated combinations of features, but the exemplified features may be unrestrictedly combined again provided that the technical problem of the invention is solved. Similarly, the features of any claim can be combined with the features of one or more other claims. The present invention having been described in summary and in detail, is illustrated and not limited by the following examples.

### Examples

### Example 1: Selection of Adenosine-Rich and Adenosine-Low Tumor Models

To select appropriate in vivo models for testing adenosine inhibition, levels of adenosine and AMP were measured from 4T1 and MC38 tumors. BALB/c mice were inoculated in the right mammary fat pad with 5×10⁴ 4T1 cells in 0.1 mL of PBS, and C57BL/6 mice were inoculated sc into the right flank with 1×10⁶ MC38 cells in 0.1 mL of PBS. Tumors were collected, snap frozen, and 50 mg of tissue was used to measure intra-tumoral adenosine and AMP concentrations by Capillary Electrophoresis Time-of-Flight Mass Spectrometry (CE-TOFMS) and Capillary Electrophoresis-Triple Quadrupole Mass Spectrometry (CE-QqQMS). The average concentrations of AMP and adenosine were 850.4±215.6 (±SD) nM/g and 87.2±51.9 nM/g for MC38 model, and 296.5±250.5 nM/g and 210.2±158.2 nM/g for 4T1 model, respectively (see FIGS. 3 A and B).

To determine whether AMP and adenosine concentrations correspond to extracellular expression of CD73 (enzyme degrading AMP down to adenosine) in MC38 and 4T1 tumors, flow cytometry analysis was conducted with the cell lines. Expression of the enzyme was quantified as CD73 copy numbers per cell based on the binding capacity of anti-CD73 staining antibodies established using the beads from the Quantum™ Simply Cellular® kit. 4T1 tumor cells expressed higher level of CD73 compared to MC38 tumor cells (128,106.07 and 6174.57 of the CD73 copy numbers/cell, respectively) (see FIGS. 3 C and D). Thus, 4T1 tumors express high levels of CD73, efficiently convert AMP into adenosine resulting in the accumulation of high levels of adenosine within the tumor tissue. Conversely, MC38 tumors, express low levels of CD73 and contain higher AMP and lower adenosine levels, likely due to slower conversion of AMP into adenosine by CD73.

In addition, extracellular CD73 protein level was assessed by flow cytometry in five additional syngeneic murine tumor cell lines: EMT6 and E0771 mammary, MBT2 bladder, H22 hepatocellular carcinoma cell lines. Expression of CD73 in EMT6 and E0771 tumor cell lines (showing 68,588.45 and 47,966.98 CD73 copy numbers per cell, respectively) was lower compared to 4T1 tumor cells, but higher compared to MC38 tumor cells (see FIGS. 3 E and F). No significant level of CD73 expression was detected in MBT2 and H22 tumor cells (see FIGS. 3 G and H).

### Example 2: The Dual Inhibition of PD-1 and TGFβ Increases the Expression of the Adenosine Receptor A_{2B} in an Adenosine-Rich Tumor Model

To determine whether bintrafusp alfa treatment modulates expression of A_{2A} or A_{2B} receptors or NT5E (gene encoding CD73), mice with established 4T1 and MC38 tumors were treated with 20 mg/kg isotype antibody or 24.6 mg/kg bintrafusp alfa. RNAseq analysis of tumors on Day 6 post treatment revealed no changes on the expression of NT5E (CD73) or A_{2A} in both models (see FIGS. 4 A and B). In contrast, as shown in FIG. 4 C, the expression of A_{2B} was increased by treatment with bintrafusp alfa in the CD73^{hi} 4T1 model, but not in CD73^{low} MC38 model, suggesting that A_{2B} expression, which increases after treatment with bintrafusp alfa, may potentially limit therapeutic efficacy of bintrafusp alfa in an adenosine-rich environment.

### Example 3: The Co-Inhibition of PD-1, TGFβ and Adenosine Signaling Synergistically Reduces the Tumor Volume in Adenosine-Rich Tumor Models

To determine whether combined treatment with the dual A_{2A}/A_{2B} receptor inhibitor “Compound A” ((S)-7-Oxa-2-aza-spiro[4.5]decane-2-carboxylic acid [7-(3,6-dihydro-2H-pyran-4-yl)-4-methoxy-thiazolo[4,5-c]pyridin-2-yl]-amide) and bintrafusp alfa can inhibit tumor growth in either CD73^{hi} or CD73^{low} models, tumor growth was monitored in seven syngeneic tumor models. Animals were inoculated with either CD73^{hi} tumor cells (4T1, E0771, or EMT6) or with CD73^{low} MC38, H22 or MBT2 tumor cells. For all tumor models, animals were treated with either 1) vehicle and isotype, 2) Compound A and isotype, 3) vehicle and bintrafusp alfa, or 4) Compound A and bintrafusp alfa.

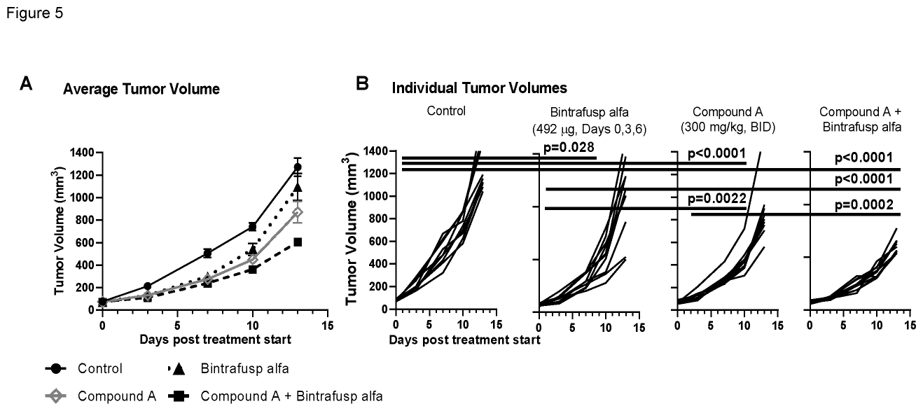

In the CD73^{hi} 4T1 tumor model, female BALB/c mice were inoculated with 5×10⁴ 4T1 cells into the right mammary fat pad and were treated with Compound A (300 mg/kg po, bid), bintrafusp alfa (24.6 mg/kg iv, days 0, 3, 6), Compound A+bintrafusp alfa, when average tumor volume reached approximately 60 mm³. Vehicle (po, bid) and isotype control antibody injections (20 mg/kg iv, days 0, 3, 6) were used as controls. As reflected by FIG. 5, treatment with bintrafusp alfa induced marginal, but statistically significant tumor growth inhibition (T/C=86.2% and p=0.0286) on Day 13 post treatment initiation compared to vehicle control. Compound A induced significantly stronger tumor growth inhibition (T/C=68.5%) with p=0.0022 and p<0.0001, when compared to bintrafusp alfa or vehicle control, respectively. However, the strongest tumor growth inhibition was detected in mice treated with a combination of Compound A with bintrafusp alfa (T/C=47.7%), which was statistically higher compared to treatment with Compound A (p=0.0002) or bintrafusp alfa (p<0.0001) alone.

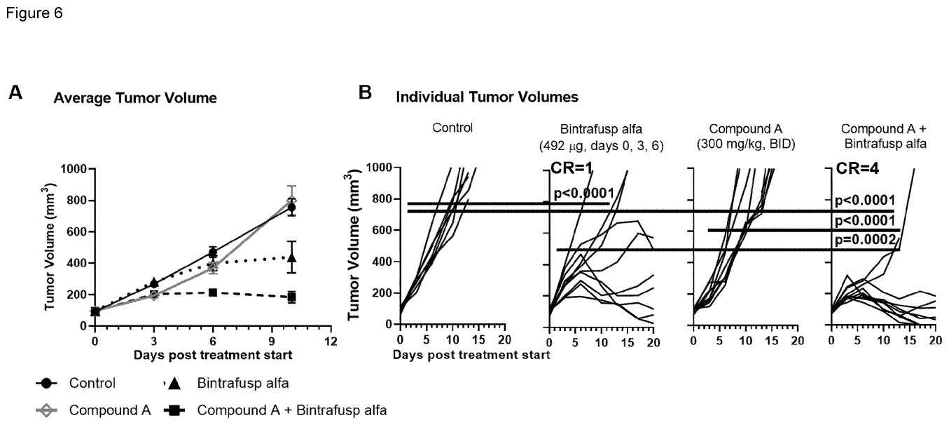

In the second CD73^{hi} EMT6 tumor model, female BALB/c mice were inoculated with 2.5×10⁵ EMT6 cells into the right mammary fat pad and were treated with Compound A (300 mg/kg po, BID), bintrafusp alfa (24.6 mg/kg iv, days 0, 3, 6), Compound A+bintrafusp alfa, when average tumor volume reached approximately 60 mm³. Vehicle (po, BID) and isotype control antibody injections (20 mg/kg iv, days 0, 3, 6) were used as controls. In this model, Compound A alone did not show tumor growth inhibition on Day 10 post treatment initiation, but significantly enhanced tumor growth inhibition in combination with bintrafusp alfa resulting in four mice with CR (complete responses) (T/C=24.4%, p<0.0001 compared to vehicle control and Compound A monotherapy, or p=0.0002 compared to bintrafusp alfa monotherapy) (see FIG. 6).

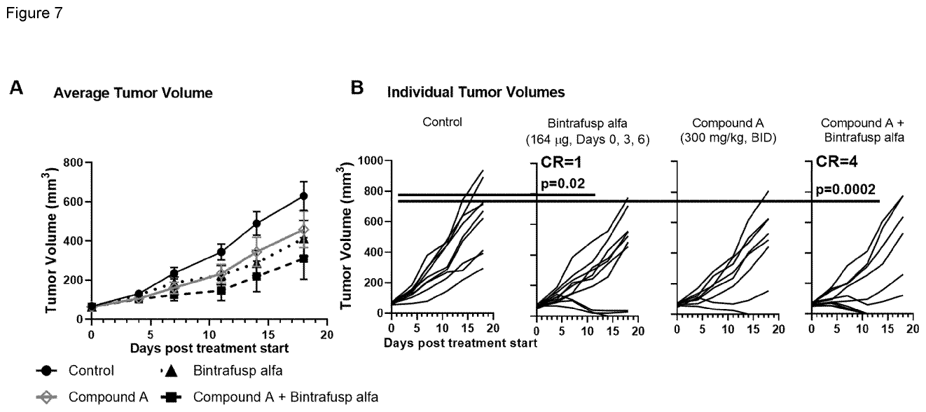

In the third CD73^{hi} tumor model, female C57BL/6 mice were inoculated with 1.5×10⁵ E0771 cells into the right mammary fat pad and were randomized into treatment groups when average tumor volume reached approximately 75 mm³. The E0771 tumor model shows high sensitivity to the original 24.6 mg/kg (iv, days 0, 3, and 6) dose of bintrafusp alfa. Thus, to test its combination potential with Compound A in this model we had to reduce the dose for bintrafusp alfa from 24 mg/kg (iv, days 0, 3, and 6) down to 8.2 mg/kg (iv, days 0, 3, 6). Thus, E0771 tumor-bearing mice were treated with Compound A (300 mg/kg po, BID), bintrafusp alfa (8.2 mg/kg iv, days 0, 3, 6), Compound A+bintrafusp alfa starting the day 0 (when they were randomized into treatment groups). Vehicle (po, BID) and isotype control antibody injections (6.65 mg/kg iv, days 0, 3, 6) were used as controls. Even at a lower dose (8.2 mg/kg, iv, days 0, 3, 6) bintrafusp alfa as monotherapy induced significant tumor growth inhibition in this model on Day 18 post treatment initiation (T/C=65.7%, p=0.02) (see FIG. 7). Although, Compound A (300 mg/kg, po, BID) as monotherapy also induced tumor growth inhibition with 1 CR (T/C=72.9%), the difference did not reach statistical significance compared to vehicle control (p=0.1348) and was not different compared to bintrafusp alfa only treated animals (p=0.93). However, Compound A significantly increased tumor growth inhibition in combination with bintrafusp alfa, resulting in 4 mice with CR (T/C=49.3%, p=0.0002 compared to control group).

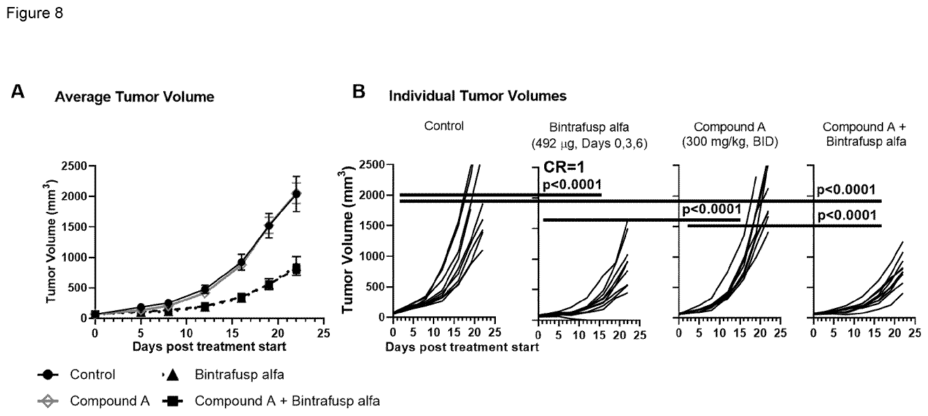

To further confirm that combination potential for Compound A with bintrafusp alfa depends on the adenosine level in the tumor, three additional syngeneic tumor models with lower CD73 expression (MC38, H22 and MBT2) were used (see FIG. 3). In the MC38 tumor model, female C57BL/6 mice were inoculated with 1×10⁶ MC38 cells in the right lower flank and were treated with Compound A (300 mg/kg po, BID), bintrafusp alfa (24.6 mg/kg iv, days 0, 3, 6), Compound A+bintrafusp alfa, when the average tumor volume reached approximately 70 mm³. Vehicle (po, BID) and isotype control antibody injections (20 mg/kg iv, days 0, 3, 6) were used as controls. There was no tumor growth inhibition observed following treatment with Compound A monotherapy in this tumor model. Combined treatment of mice with Compound A and bintrafusp alfa resulted in significant tumor growth inhibition (T/C=39.9%, p<0.0001), but did not provide any significant additional benefit relative to treatment with bintrafusp alfa alone (T/C=42.1%, p=0.98) on Day 22 post treatment initiation (see FIG. 8).

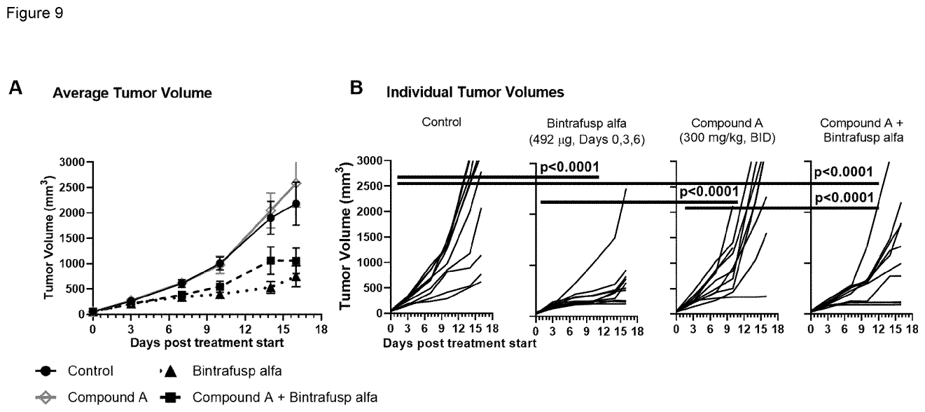

In the second the CD73′ H22 tumor model female BALB/c mice were inoculated with 1×10⁶ H22 cells in the right upper flank and were treated with Compound A (300 mg/kg po, BID), bintrafusp alfa (24.6 mg/kg iv, days 0, 3, 6), Compound A+bintrafusp alfa, when average tumor volume reached approximately 55 mm3. Vehicle (po, BID) and isotype control antibody injections (20 mg/kg iv, days 0, 3, 6) were used as controls. In H22 tumor-bearing mice, combined treatment with Compound A and bintrafusp alfa resulted in tumor growth inhibition (T/C=48.3%, p<0.0001) on Day 16 post treatment initiation, that was not statistically different from the treatment with bintrafusp alfa alone (T/C=34.4%, p=0.6015) (see FIG. 9).

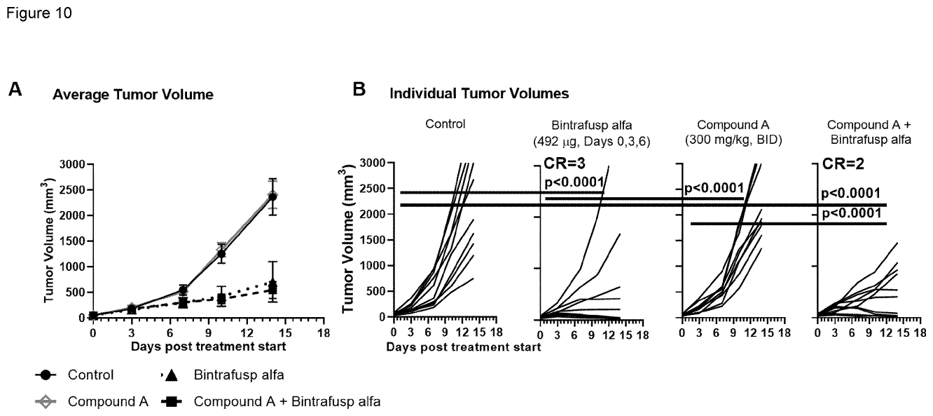

Finally, in the third CD73¹′ MBT2 tumor model, female C3H mice were inoculated with 1×10⁶ MBT2 cells in the right upper flank and were treated with Compound A (300 mg/kg po, BID), bintrafusp alfa (24.6 mg/kg iv, days 0, 3, 6), Compound A+bintrafusp alfa, when average tumor volume reached approximately 53 mm³. Vehicle (po, BID) and isotype control antibody injections (20 mg/kg iv, days 0, 3, 6) were used as controls. Similar to CD73¹′ MC38 and H22 tumor models, in MBT2 tumor bearing mice, combined treatment with Compound A and bintrafusp alfa resulted in tumor growth inhibition (T/C=23.1%, p<0.0001 relative to control group on Day 14) that was not statistically different relative to bintrafusp alfa alone (T/C=29.9%, p=0.9574) (see FIG. 10).

Together, these data suggest that in CD73^{hi}, i.e. adenosine-rich, but not CD73^{low}, tumor models, the co-inhibition of PD-1, TGFβ and adenosine signaling shows significant tumor growth inhibition.

### Example 4: The Co-Inhibition of PD-1, TGFβ and Adenosine Signaling Synergistically Rescue Production of IFNγ from Human T Cells Co-Cultured with Tumor Cells in the Presence of the Stable Adenosine Analogue NECA

The ability of Compound A alone or in combination with bintrafusp alfa to protect human T cell activation from adenosine-driven suppression was tested in an in vitro assay with EBV-positive human PBMCs co-cultured with MDA-MB-231 human breast cancer cells. This assay was set-up by co-culturing MDA-MB-231 tumor cells loaded with EBV LMP-2 peptide and EBV-specific T cells pre-treated with Compound A or in combination with bintrafusp alfa or isotype control antibodies in presence of NECA.

Briefly, MDA-MB-231 tumor cells were seeded to the flat bottom 96-well plate at 2.6×10⁴ cells/well (in 100 μl of RPMI1640 media, supplemented with 10% FBS) and loaded with EBV peptide (30 ng/ml, CLGGLLTMV, 21^{st} Century). EBV-specific T cells were pre-incubated in round bottom 96-well plate with Compound A (100 nM) and/or 1 μg/ml of bintrafusp alfa or isotype control (hIgG1 inactive anti-PD-L1) antibodies for 15 minutes. Then 10 μM NECA or DMSO control was added to the cells and 1.3×10⁴ of the T cells (in 100 μl volume) were transferred per well to the plate with peptide-loaded MDA-MB-231 tumor cells at a T cell to tumor cell ratio of 0.5:1. Cell culture supernatants were collected after 74 hours of co-culture, and IFNγ levels were measured using human IFNγ ELISA Kit (R&D Systems) according to manufacturer instructions. The percentage of IFNγ secretion rescue was calculated using the following formula: 100%−(IFNγ in samples treated with NECA and Compound A alone or combination with bintrafusp alfa minus IFNγ in control sample)+(IFNγ in samples with NECA minus IFNγ in control sample)*100%.

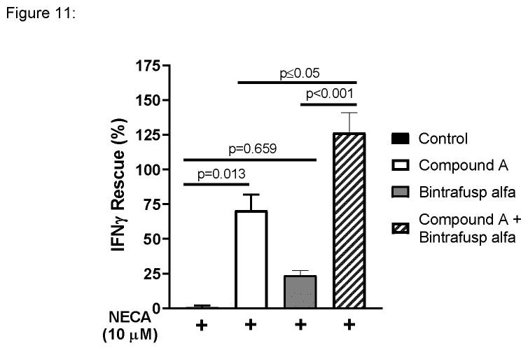

In this assay, Compound A, but not bintrafusp alfa as monotherapy significantly rescued IFNγ secretion from human T cells (approx. 71% and 24%, p=0.01 and p=0.66 for Compound A and Bintrafusp alfa monotherapies respectively, compared to the T cells co-cultured with MDA-MB-231 tumor cells in presence of NECA and isotype control antibodies only). At the same time combination treatment with Compound A and bintrafusp alfa in this assay resulted in maximal (˜127%) rescue of IFNγ secretion, which was significantly stronger compared to Compound A or bintrafusp alfa monotherapies (p≤0.05 and p<0.001, respectively) (FIG. 11).

### Example 5: The Co-Inhibition of PD-1, TGFβ and Adenosine Signaling does not Synergistically Reduce the Tumor Volume in a CD73-KO Tumor Model

Further to the results of Example 3, tumor growth inhibition by the combined treatment with the dual A2A/A2B receptor inhibitor “Compound A” ((S)-7-Oxa-2-aza-spiro[4.5]decane-2-carboxylic acid [7-(3,6-dihydro-2H-pyran-4-yl)-4-methoxy-thiazolo[4,5-c]pyridin-2-yl]-amide) and bintrafusp alfa was examined in a syngeneic 4T1 tumor model, in which CD73 was knocked-out using GenCRISPR™ gene editing technology (GenScript USA, Inc). Efficacy of CD73 knockout in 4T1 tumor cells was confirmed by flow cytometry. Female BALB/c mice were inoculated into the right mammary fat pad with 1×10⁵ CD73 KO 4T1 tumor cells and were treated with Compound A (300 mg/kg po, bid), bintrafusp alfa (24.6 mg/kg iv, days 0, 3, 6), Compound A+bintrafusp alfa, when average tumor volume reached approximately 60 mm³. Vehicle (po, bid) and isotype control antibody injections (20 mg/kg iv, days 0, 3, 6) were used as controls, i.e., animals were treated with either 1) vehicle and isotype, 2) Compound A and isotype, 3) vehicle and bintrafusp alfa, or 4) Compound A and bintrafusp alfa.

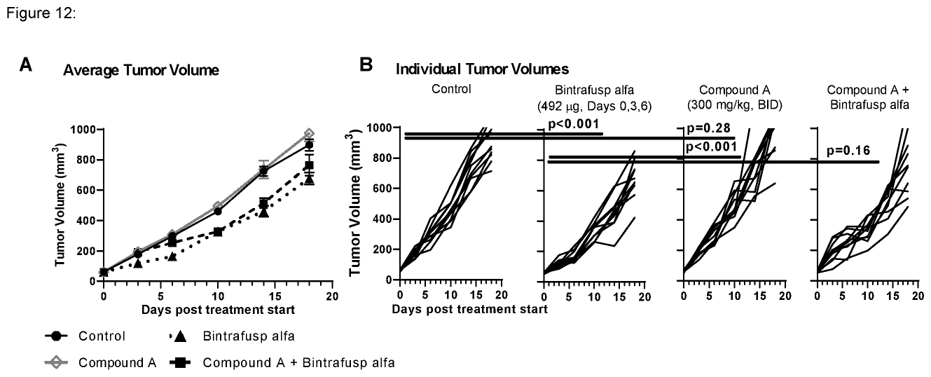

As reflected by FIG. 12, treatment with bintrafusp alfa induced statistically significant tumor growth inhibition (T/C=75.4% and p<0.001) on Day 18 post treatment initiation compared to vehicle control. In contrast, Compound A did not induce tumor growth inhibition (T/C=108%) when compared to vehicle control. Although, combination of Compound A with bintrafusp alfa showed statistically significant tumor growth inhibition compared to vehicle treated control mice in this CD73 KO 4T1 tumor model (T/C=85.2% and p<0.001), that was not statistically different from the treatment with bintrafusp alfa alone (p=0.16) (see FIG. 12).

SEQUENCE LISTINGS

| SEQ | | |
|---|---|---|
| ID NO. | Sequence | Description |
| 1 | SYIMM | Bintrafusp alfa CDRH1 |
| 2 | SIYPSGGITFYADTVKG | Bintrafusp alfa CDRH2 |
| 3 | IKLGTVTTVDY | Bintrafusp alfa CDRH3 |
| 4 | TGTSSDVGGYNYVS | Bintrafusp alfa CDRL1 |
| 5 | DVSNRPS | Bintrafusp alfa CDRL2 |
| 6 | SSYTSSSTRV | Bintrafusp alfa CDRL3 |
| 7 | QSALTQPASVSGSPGQSITISCTGTSSDVGGYNYVSWYQQHP | Bintrafusp alfa light chain |
| | GKAPKLMIYDVSNRPSGVSNRFSGSKSGNTASLTISGLQAEDE | |
| | ADYYCSSYTSSSTRVFGTGTKVTVLGQPKANPTVTLFPPSSEE | |
| | LQANKATLVCLISDFYPGAVTVAWKADGSPVKAGVETTKPSK | |
| | QSNNKYAASSYLSLTPEQWKSHRSYSCQVTHEGSTVEKTVAP | |
| | TECS | |
| 8 | EVQLLESGGGLVQPGGSLRLSCAASGFTFSSYIMMWVRQAP | Bintrafusp alfa heavy |
| | GKGLEWVSSIYPSGGITFYADTVKGRFTISRDNSKNTLYLQMN | chain |
| | SLRAEDTAVYYCARIKLGTVTTVDYWGQGTLVTVSSASTKGPS | |
| | VFPLAPSSKSTSGGTAALGCLVKDYFPEPVTVSWNSGALTSG | |
| | VHTFPAVLQSSGLYSLSSVVTVPSSSLGTQTYICNVNHKPSNT | |
| | KVDKRVEPKSCDKTHTCPPCPAPELLGGPSVFLFPPKPKDTL | |
| | MISRTPEVTCVVVDVSHEDPEVKFNWYVDGVEVHNAKTKPRE | |
| | EQYNSTYRVVSVLTVLHQDWLNGKEYKCKVSNKALPAPIEKTI | |
| | SKAKGQPREPQVYTLPPSREEMTKNQVSLTCLVKGFYPSDIA | |
| | VEWESNGQPENNYKTTPPVLDSDGSFFLYSKLTVDKSRWQQ | |
| | GNVFSCSVMHEALHNHYTQKSLSLSPGAGGGGSGGGGSGG | |
| | GGSGGGGSGIPPHVQKSVNNDMIVTDNNGAVKFPQLCKFCD | |
| | VRFSTCDNQKSCMSNCSITSICEKPQEVCVAVWRKNDENITLE | |
| | TVCHDPKLPYHDFILEDAASPKCIMKEKKKPGETFFMCSCSSD | |
| | ECNDNIIFSEEYNTSNPD | |
| 9 | MGRGLLRGLWPLHIVLWTRIASTIPPHVQKSDVEMEAQKDEIIC | TGFβRII isoform A |
| | PSCNRTAHPLRHINNDMIVTDNNGAVKFPQLCKFCDVRFSTC | |
| | DNQKSCMSNCSITSICEKPQEVCVAVWRKNDENITLETVCHDP | |
| | KLPYHDFILEDAASPKCIMKEKKKPGETFFMCSCSSDECNDNII | |
| | FSEEYNTSNPDLLLVIFQVTGISLLPPLGVAISVIIIFYCYRVNRQ | |
| | QKLSSTWETGKTRKLMEFSEHCAIILEDDRSDISSTCANNINHN | |
| | TELLPIELDTLVGKGRFAEVYKAKLKQNTSEQFETVAVKIFPYE | |
| | EYASWKTEKDIFSDINLKHENILQFLTAEERKTELGKQYWLITA | |
| | FHAKGNLQEYLTRHVISWEDLRKLGSSLARGIAHLHSDHTPCG | |
| | RPKMPIVHRDLKSSNILVKNDLTCCLCDFGLSLRLDPTLSVDDL | |
| | ANSGQVGTARYMAPEVLESRMNLENVESFKQTDVYSMALVL | |
| | WEMTSRCNAVGEVKDYEPPFGSKVREHPCVESMKDNVLRDR | |
| | GRPEIPSFWLNHQGIQMVCETLTECWDHDPEARLTAQCVAER | |
| | FSELEHLDRLSGRSCSEEKIPEDGSLNTTK | |
| 10 | MGRGLLRGLWPLHIVLWTRIASTIPPHVQKSVNNDMIVTDNNG | TGFβRII isoform B |
| | AVKFPQLCKFCDVRFSTCDNQKSCMSNCSITSICEKPQEVCVA | |
| | VWRKNDENITLETVCHDPKLPYHDFILEDAASPKCIMKEKKKP | |
| | GETFFMCSCSSDECNDNIIFSEEYNTSNPDLLLVIFQVTGISLLP | |
| | PLGVAISVIIIFYCYRVNRQQKLSSTWETGKTRKLMEFSEHCAII | |
| | LEDDRSDISSTCANNINHNTELLPIELDTLVGKGRFAEVYKAKL | |
| | KQNTSEQFETVAVKIFPYEEYASWKTEKDIFSDINLKHENILQFL | |
| | TAEERKTELGKQYWLITAFHAKGNLQEYLTRHVISWEDLRKLG | |
| | SSLARGIAHLHSDHTPCGRPKMPIVHRDLKSSNILVKNDLTCCL | |
| | CDFGLSLRLDPTLSVDDLANSGQVGTARYMAPEVLESRMNLE | |
| | NVESFKQTDVYSMALVLWEMTSRCNAVGEVKDYEPPFGSKV | |
| | REHPCVESMKDNVLRDRGRPEIPSFWLNHQGIQMVCETLTEC | |
| | WDHDPEARLTAQCVAERFSELEHLDRLSGRSCSEEKIPEDGS | |
| | LNTTK | |
| 11 | IPPHVQKSVNNDMIVTDNNGAVKFPQLCKFCDVRFSTCDNQK | TGFβRII extracellular |
| | SCMSNCSITSICEKPQEVCVAVWRKNDENITLETVCHDPKLPY | domain fragment |
| | HDFILEDAASPKCIMKEKKKPGETFFMCSCSSDECNDNIIFSEE | |
| | YNTSNPD | |
| 12 | GAVKFPQLCKFCDVRFSTCDNQKSCMSNCSITSICEKPQEVC | TGFβRII extracellular |
| | VAVWRKNDENITLETVCHDPKLPYHDFILEDAASPKCIMKEKK | domain fragment |
| | KPGETFFMCSCSSDECNDNIIFSEEYNTSNPD | |
| 13 | VKFPQLCKFCDVRFSTCDNQKSCMSNCSITSICEKPQEVCVAV | TGFβRII extracellular |
| | WRKNDENITLETVCHDPKLPYHDFILEDAASPKCIMKEKKKPG | domain fragment |
| | ETFFMCSCSSDECNDNIIFSEEYNTSNPD | |
| 14 | QVQLVQSGAEVKKPGASVKVSCKASGYTFTSYWMHWVRQA | Anti-PD-L1 antibody |
| | PGQGLEWMGRIGPNSGFTSYNEKFKNRVTMTRDTSTSTVYM | heavy chain |
| | ELSSLRSEDTAVYYCARGGSSYDYFDYWGQGTTVTVSSASTK | |
| | GPSVFPLAPCSRSTSESTAALGCLVKDYFPEPVTVSWNSGAL | |
| | TSGVHTFPAVLQSSGLYSLSSVVTVPSSSLGTKTYTCNVDHKP | |
| | SNTKVDKRVESKYGPPCPPCPAPEAAGGPSVFLFPPKPKDTL | |
| | MISRTPEVTCVVVDVSQEDPEVQFNWYVDGVEVHNAKTKPRE | |
| | EQFNSTYRVVSVLTVLHQDWLNGKEYKCKVSNKGLPSSIEKTI | |
| | SKAKGQPREPQVYTLPPSQEEMTKNQVSLTCLVKGFYPSDIA | |
| | VEWESNGQPENNYKTTPPVLDSDGSFFLYSRLTVDKSRWQE | |
| | GNVFSCSVMHEALHNHYTQKSLSLSLGK | |
| 15 | DIVLTQSPASLAVSPGQRATITCRASESVSIHGTHLMHWYQQK | Anti-PD-L1 antibody light |
| | PGQPPKLLIYAASNLESGVPARFSGSGSGTDFTLTINPVEAED | chain |
| | TANYYCQQSFEDPLTFGQGTKLEIKRTVAAPSVFIFPPSDEQL | |
| | KSGTASVVCLLNNFYPREAKVQWKVDNALQSGNSQESVTEQ | |
| | DSKDSTYSLSSTLTLSKADYEKHKVYACEVTHQGLSSPVTKSF | |
| | NRGEC | |
| 16 | EVQLLESGGGLVQPGGSLRLSCAASGFTFSSYIMMWVRQAP | Anti-PD-L1 antibody |
| | GKGLEWVSSIYPSGGITFYADTVKGRFTISRDNSKNTLYLQMN | heavy chain |
| | SLRAEDTAVYYCARIKLGTVTTVDYWGQGTLVTVSSASTKGPS | |
| | VFPLAPSSKSTSGGTAALGCLVKDYFPEPVTVSWNSGALTSG | |
| | VHTFPAVLQSSGLYSLSSVVTVPSSSLGTQTYICNVNHKPSNT | |
| | KVDKRVEPKSCDKTHTCPPCPAPELLGGPSVFLFPPKPKDTL | |
| | MISRTPEVTCVVVDVSHEDPEVKFNWYVDGVEVHNAKTKPRE | |
| | EQYNSTYRVVSVLTVLHQDWLNGKEYKCKVSNKALPAPIEKTI | |
| | SKAKGQPREPQVYTLPPSREEMTKNQVSLTCLVKGFYPSDIA | |
| | VEWESNGQPENNYKTTPPVLDSDGSFFLYSKLTVDKSRWQQ | |
| | GNVFSCSVMHEALHNHYTQKSLSLSPGK | |
| 17 | QVQLVQSGAEVKKPGASVKVSCKASGYTFTSYWMHWVRQA | Anti-PD-L1: TGFβRII |
| | PGQGLEWMGRIGPNSGFTSYNEKFKNRVTMTRDTSTSTVYM | fusion protein heavy chain |
| | ELSSLRSEDTAVYYCARGGSSYDYFDYWGQGTTVTVSSASTK | as disclosed in WO |
| | GPSVFPLAPCSRSTSESTAALGCLVKDYFPEPVTVSWNSGAL | 2018/205985 |
| | TSGVHTFPAVLQSSGLYSLSSVVTVPSSSLGTKTYTCNVDHKP | |
| | SNTKVDKRVESKYGPPCPPCPAPEAAGGPSVFLFPPKPKDTL | |
| | MISRTPEVTCVVVDVSQEDPEVQFNWYVDGVEVHNAKTKPRE | |
| | EQFNSTYRVVSVLTVLHQDWLNGKEYKCKVSNKGLPSSIEKTI | |
| | SKAKGQPREPQVYTLPPSQEEMTKNQVSLTCLVKGFYPSDIA | |
| | VEWESNGQPENNYKTTPPVLDSDGSFFLYSRLTVDKSRWQE | |
| | GNVFSCSVMHEALHNHYTQKSLSLSLGAGGGGGGGGSGG | |
| | GGSGGGGSGGAVKFPQLCKFCDVRFSTCDNQKSCMSNCSIT | |
| | SICEKPQEVCVAVWRKNDENITLETVCHDPKLPYHDFILEDAA | |
| | SPKCIMKEKKKPGETFFMCSCSSDECNDNIIFSEEYNTSNPD | |
| 18 | QVQLVQSGAEVKKPGASVKVSCKASGYTFTSYWMHWVRQA | Anti-PD-L1: TGFβRII |
| | PGQGLEWMGRIGPNSGFTSYNEKFKNRVTMTRDTSTSTVYM | fusion protein heavy chain |
| | ELSSLRSEDTAVYYCARGGSSYDYFDYWGQGTTVTVSSASTK | as disclosed in WO |
| | GPSVFPLAPCSRSTSESTAALGCLVKDYFPEPVTVSWNSGAL | 2018/205985 |
| | TSGVHTFPAVLQSSGLYSLSSVVTVPSSSLGTKTYTCNVDHKP | |
| | SNTKVDKRVESKYGPPCPPCPAPEAAGGPSVFLFPPKPKDTL | |
| | MISRTPEVTCVVVDVSQEDPEVQFNWYVDGVEVHNAKTKPRE | |
| | EQFNSTYRVVSVLTVLHQDWLNGKEYKCKVSNKGLPSSIEKTI | |
| | SKAKGQPREPQVYTLPPSQEEMTKNQVSLTCLVKGFYPSDIA | |
| | VEWESNGQPENNYKTTPPVLDSDGSFFLYSRLTVDKSRWQE | |
| | GNVFSCSVMHEALHNHYTQKSLSLSLGAGGGGGGGGSGG | |
| | GGSGGGGSGGGGSGVKFPQLCKFCDVRFSTCDNQKSCMSN | |
| | CSITSICEKPQEVCVAVWRKNDENITLETVCHDPKLPYHDFILE | |
| | DAASPKCIMKEKKKPGETFFMCSCSSDECNDNIIFSEEYNTSN | |
| | PD | |
| 19 | SYWMH | CDRH1 of anti-PD-L1 |
| | | antibody as disclosed in |
| | | WO 2018/205985 |
| 20 | RIX1PNSGX2TSYNEKFKN, wherein X1 is H or G and wherein | CDRH2 of anti-PD-L1 |
| | X2 is G or F | antibody as disclosed in |
| | | WO 2018/205985 |
| 21 | GGSSYDYFDY | CDRH3 of anti-PD-L1 |
| | | antibody as disclosed in |
| | | WO 2018/205985 |
| 22 | RASESVSIHGTHLMH | CDRL1 of anti-PD-L1 |
| | | antibody as disclosed in |
| | | WO 2018/205985 |
| 23 | AASNLES | CDRL2 of anti-PD-L1 |
| | | antibody as disclosed in |
| | | WO 2018/205985 |
| 24 | QQSFEDPLT | CDRL3 of anti-PD-L1 |
| | | antibody as disclosed in |
| | | WO 2018/205985 |
| 25 | QSALTQPASVSGSPGQSITISCTGTSSDVGGYNYVSWYQQHP | Bintrafusp alfa light chain |
| | GKAPKLMIYDVSNRPSGVSNRFSGSKSGNTASLTISGLQAEDE | variable region |
| | ADYYCSSYTSSSTRVFGTGTKVTVL | |
| 26 | EVQLLESGGGLVQPGGSLRLSCAASGFTFSSYIMMWVRQAP | Bintrafusp alfa heavy |
| | GKGLEWVSSIYPSGGITFYADTVKGRFTISRDNSKNTLYLQMN | chain variable region |
| | SLRAEDTAVYYCARIKLGTVTTVDYWGQGTLVTVSS | |

1. A method of treating a cancer in a subject,
wherein the method comprises administering a PD-1 inhibitor, a TGFβ inhibitor and an adenosine A_{2A} and/or A_{2B} receptor inhibitor to the subject.

2. The method according to claim 1, wherein the PD-1 inhibitor is an anti-PD-L1 antibody, or a fragment thereof capable of binding PD-L1, and the TGFβ inhibitor is a TGFβRII, or a fragment thereof capable of binding TGF-β, or an anti-TGFβ antibody, or a fragment thereof capable of binding TGFβ.

3. The method according to claim 1, wherein the anti-PD-L1 antibody or fragment thereof comprises a heavy chain sequence, which comprises a CDRH1 having the sequence of SEQ ID NO: 1, a CDRH2 having the sequence of SEQ ID NO: 2 and a CDRH3 having the sequence of SEQ ID NO: 3, and a light chain sequence, which comprises a CDRL1 having the sequence of SEQ ID NO: 4, a CDRL2 having the sequence of SEQ ID NO: 5 and a CDRL3 having the sequence of SEQ ID NO: 6; or
wherein the anti-PD-L1 antibody or fragment thereof comprises a heavy chain sequence, which comprises a CDRH1 having the sequence of SEQ ID NO: 19, a CDRH2 having the sequence of SEQ ID NO: 20 and a CDRH3 having the sequence of SEQ ID NO: 21, and a light chain sequence, which comprises a CDRL1 having the sequence of SEQ ID NO: 22, a CDRL2 having the sequence of SEQ ID NO: 23 and a CDRL3 having the sequence of SEQ ID NO: 24.

4. The method according to claim 1, wherein the TGFβ inhibitor is an extracellular domain of TGFβRII or a fragment thereof capable of binding TGFβ.

5. The method according to claim 1, wherein the PD-1 inhibitor and the TGFβ inhibitor are fused as an anti-PD(L)1:TGFβRII fusion protein.

6. The method according to claim 5, wherein the light chain sequences and the heavy chain sequences of the anti-PD(L)1:TGFβRII fusion protein have at least 90% sequence identity to the light chain sequence and the heavy chain sequence selected from the group consisting of: (1) SEQ ID NO: 7 and SEQ ID NO: 8, (2) SEQ ID NO: 15 and SEQ ID NO: 17, and (3) SEQ ID NO: 15 and SEQ ID NO: 18.

7. The compounds-for-use method according to claim 5, wherein the amino acid sequence of the anti-PD(L)1:TGFβRII fusion protein corresponds to the amino acid sequence of bintrafusp alfa.

8. The method according to claim 5, wherein the anti-PD(L)1:TGFβRII fusion protein is administered at a dose of 1200 mg Q2W or at a dose of 2400 mg Q3W.

9. The method according to claim 1, wherein the adenosine A_{2A} and/or A_{2B} receptor inhibitor is one of the compounds selected from Tables 1 and 2.

10. The compounds-for-use method according to claim 9, wherein the adenosine A_{2A} and/or A_{2B} receptor inhibitor is (S)-7-Oxa-2-aza-spiro[4.5]decane-2-carboxylic acid [7-(3,6-dihydro-2H-pyran-4-yl)-4-methoxy-thiazolo[4,5-c]pyridin-2-yl]-amide or a pharmaceutically acceptable salt, derivative, solvate, prodrug and stereoisomer thereof, including mixtures thereof in all ratios.

11. The method according to claim 9, wherein the adenosine A_{2A} and/or A_{2B} receptor inhibitor is administered orally at a dose of 50-150 mg BID.

12. A method of treating a cancer in a subject, wherein the method comprises administering a PD-1 inhibitor, a TGFβ inhibitor and an adenosine A_{2A} and/or A_{2B} receptor inhibitor to the subject; and wherein the PD-1 inhibitor and TGFβ inhibitor are fused in a molecule having the amino acid sequence of bintrafusp alfa and the adenosine A_{2A} and/or A_{2B} receptor inhibitor is (S)-7-Oxa-2-aza-spiro[4.5]decane-2-carboxylic acid [7-(3,6-dihydro-2H-pyran-4-yl)-4-methoxy-thiazolo[4,5-c]pyridin-2-yl]-amide or a pharmaceutically acceptable salt, derivative, solvate, prodrug and stereoisomer thereof, including mixtures thereof in all ratios.

13. The method according to claim 1, wherein the cancer is CD73 positive and/or adenosine-rich.

14. The method according to claim 13, wherein the adenosine-rich cancer has adenosine levels that are sufficient for adenosine A_{2B} receptor-mediated signaling.

15. The method of claim 3, wherein the cancer is CD73 positive and/or adenosine-rich.

16. The method of claim 15, wherein the adenosine-rich cancer has adenosine levels that are sufficient for adenosine A_{2B} receptor-mediated signaling.

17. The method of claim 12, wherein the cancer is CD73 positive and/or adenosine-rich.

18. The method of claim 17, wherein the adenosine-rich cancer has adenosine levels that are sufficient for adenosine A_{2B} receptor-mediated signaling.
